(19) United States
(12) Patent Application Publication
(10) Pub. No.: US 2024/0423511 A1
(43) Pub. Date: Dec. 26, 2024

(54) METHODS FOR INCORPORATING THERAPEUTIC AGENTS INTO ANALYTE SENSORS

(71) Applicant: ABBOTT DIABETES CARE INC., Alameda, CA (US)

(72) Inventors: Carlo DI IULIO, Witney (GB); Harrison E. BRADBEER, Bicester (GB); Sadie H. SINCLAIR, Ickenham (GB); Simon A. HECTOR, Swindon Wiltshire (GB)

(73) Assignee: ABBOTT DIABETES CARE INC., Alameda, CA (US)

(21) Appl. No.: 18/674,805

(22) Filed: May 24, 2024

Related U.S. Application Data
(60) Provisional application No. 63/504,114, filed on May 24, 2023.
(60) Provisional application No. 63/504,097, filed on May 24, 2023.

(51) Int. Cl. A61B 5/145; A61B 5/1473

(52) U.S. Cl. CPC A61B 5/14532; A61B 5/1451; A61B 5/1473; A61B 2560/063; A61B 2562/125

(57) ABSTRACT

The present disclosure provides methods for depositing a therapeutic agent onto analyte sensors (e.g., to reduce signal inaccuracies or in vivo sensor failure, e.g., due to foreign body response (FBR)). In certain embodiments, the present disclosure relates to a process for coating (e.g., dip coating) analyte sensors with therapeutic agent-containing polymer compositions to obtain analyte sensors that include a therapeutic agent. The present disclosure further relates to sharps coated with a therapeutic agent.

### CROSS REFERENCE TO RELATED APPLICATIONS

The present application claims priority to and the benefit of U.S. Provisional Application No. 63/504,114, filed on May 24, 2023, and U.S. Provisional Application No. 63/504,097, filed on May 24, 2023, the entire content of each of which being incorporated herein by reference in its entirety.

### FIELD

The present disclosure relates to a process for depositing a therapeutic agent onto analyte sensors to reduce signal inaccuracies or in vivo sensor failure, e.g., due to foreign body response (FBR). In certain embodiments, the present disclosure relates to a process for coating (e.g., dip coating) analyte sensors into therapeutic agent-containing polymer compositions to obtain analyte sensors that include a therapeutic agent. The present disclosure further relates to sharps coated with a therapeutic agent.

### BACKGROUND

The detection of one or more suitable analytes within an individual in need can sometimes be critical for monitoring the condition of his/her health as deviations from normal analyte levels can be indicative of a physiological condition. For example, monitoring glucose levels can enable people suffering from diabetes to take appropriate or suitable corrective action including administration of medicine or consumption of particular food or beverage products to avoid significant physiological harm. Other analytes can be desirable to monitor for other physiological conditions. In certain instances, it can be desirable to monitor more than one analyte to monitor multiple physiological conditions, particularly when a person is suffering from comorbid conditions that result in simultaneous dysregulation of two or more analytes in combination with one another.

Analyte monitoring in an individual can take place periodically or continuously over a period of time. Periodic analyte monitoring can take place by withdrawing a sample of bodily fluid, such as blood or urine, at set time intervals and analyzing ex vivo. Periodic, ex vivo analyte monitoring can be sufficient to determine the physiological condition of many individuals. However, ex vivo analyte monitoring can be inconvenient or painful in some instances. Moreover, there is no way to recover lost data when an analyte measurement is not obtained at an appropriate or suitable time. Continuous analyte monitoring can be conducted utilizing one or more sensors that remain at least partially implanted within a tissue of an individual, such as dermally, subcutaneously, or intravenously, so that analyses can be conducted in vivo. Implanted sensors can collect analyte data on-demand, at a set schedule, or continuously, depending on an individual's particular health needs and/or previously measured analyte levels. Analyte monitoring with an in vivo implanted sensor can be a more desirable approach for individuals having severe analyte dysregulation and/or rapidly fluctuating analyte levels, although it can also be beneficial for other individuals as well.

However, implantable sensors with a long-term wear duration (e.g., 30-day wear duration) can be plagued by short life spans due to a late sensor attenuation (LSA) when implanted in vivo. The in vivo loss and/or reduced sensitivity of sensor function seen in implantable sensors during a long-term wear, i.e., the late sensor attenuation, is thought to be in large part the result of certain biological responses (e.g., foreign body response), including immune responses, inflammation, fibrosis, and vessel regression, that occur in the tissue around (e.g., surrounding) implanted sensor tail of the analyte sensor. These tissue responses can be the result of tissue trauma arising from the insertion of the sensor tail into the skin, and can result from the tissue reacting to the sensor tail as a foreign body. The anti-inflammatory drug, for example, dexamethasone, can be utilized to prevent or reduce the foreign body response responsible for this effect.

To allow controlled or selected release of a drug over an extended wear duration, the drug can be dissolved in a biocompatible polymer which is then applied to the sensor. However, the location of the therapeutic-containing polymer composition can affect the sensing chemistry of the analyte sensor, such as interfering with the active chemistry of the analyte sensor, affecting a sensor thickness around the sensing spot of the analyte sensor, etc. Therefore, there is a need in the art to develop a process to manufacture an analyte sensor loaded with a therapeutic-containing polymer membrane/composition in a region that will not affect the sensing chemistry, e.g., in a non-sensing region of the analyte sensor.

Further, the application method for the polymer composition should be efficient, low-wastage and adaptable to the analyte sensor high-volume manufacturing facility. However, the polymer of choice for controlled or selected drug elution, which can pair with the drug, may not be compatible or suitable for covering the sensing chemistry of the analyte sensor, such as glucose sensing chemistry. Furthermore, due to the complex and miniature configurations or geometries of the sensor tail of the analyte sensor and the relatively large deposition volume required, it poses an issue for deposition techniques. Therefore, there is also a need in the art to develop a deposition method compatible with the drug-elution polymer composition and adopted into the current high-volume manufacturing process.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by compositions, devices, and methods particularly pointed out in the written description and claims hereof, as well as from the appended drawings.

Additional aspects will be set forth in part in the description which follows and, in part, will be apparent from the description, or can be learned by practice of the presented embodiments.

The present disclosure provides methods for manufacturing an analyte sensor comprising a therapeutic agent. In certain embodiments, the method comprises: providing an analyte sensor comprising an in vivo portion configured to reside below a skin surface of a subject and in contact with interstitial fluid of the subject, wherein the in vivo portion comprises a second region comprising an active area and a first region distal to the second region; preparing a first polymer solution comprising a first polymer; preparing a second polymer solution comprising a second polymer and a therapeutic agent; dipping the in vivo portion in the first polymer solution to coat the first region and the second region to generate a first polymer membrane; and dipping the first region in the second polymer solution to coat the first region and not the second region to generate a second polymer membrane comprising the therapeutic agent on top of the first polymer membrane.

In certain embodiments, a method for manufacturing an analyte sensor comprising a therapeutic agent, where the method comprises: providing an analyte sensor comprising an in vivo portion configured to reside below a skin surface of a subject and in contact with interstitial fluid of the subject, wherein the in vivo portion comprises a second region comprising an active area and a first region distal to the second region; preparing a first polymer solution comprising a first polymer; preparing a second polymer solution comprising a second polymer and a therapeutic agent; dipping the first region in the second polymer solution to coat the first region and not the second region to generate a second polymer membrane comprising the therapeutic agent; and dipping the in vivo portion in the first polymer solution to coat the first region and the second region to generate a first polymer membrane on top of the second polymer membrane.

In certain embodiments, the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.

In certain embodiments, the anti-inflammatory agent is dexamethasone, a derivative thereof, or a salt form thereof.

In certain embodiments, the second polymer solution comprises the polymer, the therapeutic agent and a solvent. In certain embodiments, the solvent comprises an alcohol, a buffer or a combination thereof. In certain embodiments, the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof. In certain embodiments, the second polymer solution further comprises a crosslinker. In certain embodiments, the therapeutic agent is present in the second polymer solution in a range from about 0.01 wt % to about 50 wt % based on the total weight of the second polymer solution. In certain embodiments, the polymer is present in the second polymer solution in a range from about 0.01 wt % to about 50 wt % based on the total weight of the second polymer solution. In certain embodiments, the crosslinker is present in the second polymer solution in a range from about 0.01 wt % to about 50 wt % based on the total weight of the second polymer solution. In certain embodiments, the second polymer solution has a viscosity of about 20 cP to about 250 cP. In certain embodiments, the second polymer solution has a viscosity of about 10 cP to about 150 cP. In certain embodiments, the second polymer solution has a viscosity of about 20 cP to about 80 cP. In certain embodiments, the second polymer of the second polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof. In certain embodiments, the polyvinylpyridine-based polymer is a polyvinylpyridine-based copolymer. In certain embodiments, the polyvinylpyridine-based copolymer is a polyvinylpyridine-co-polystyrene polymer.

In certain embodiments, the first region is dipped in the second polymer solution for a time period of about 1 second to about 5 seconds. In certain embodiments, the method further comprising drying the second polymer solution for a time period of about 1 to about 20 minutes after dipping. In certain embodiments, the first region of the analyte sensor is dipped into the second polymer solution one or more times to generate a polymer membrane having a thickness between about 5 μm to about 40 μm. In certain embodiments, the first region of the analyte sensor is dipped into the second polymer solution at least three times. In certain embodiments, the first region is ablated or planed prior to the dipping of the first region into the second polymer solution. In certain embodiments, the second region has a length of about 5 μm to about 50 μm. In certain embodiments, the first region has a length of about 5 μm to about 50 μm. In certain embodiments, the first region has a length of about 5 μm to about 20 μm.

In certain embodiments, the active area comprises a plurality of sensing regions. In certain embodiments, at least one of the plurality of sensing regions is a spot.

In certain embodiments, the first polymer of the first polymer solution is different from the second polymer. In certain embodiments, the first polymer of the first polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof. In certain embodiments, the polyvinylpyridine-based polymer is a polyvinylpyridine-based copolymer. In certain embodiments, the polyvinylpyridine-based copolymer is a polyvinylpyridine-co-polystyrene polymer. In certain embodiments, the in vivo portion of the analyte sensor is dipped in the first polymer solution for a time period of about 1 second to about 5 seconds. In certain embodiments, the method further comprising drying the first polymer solution for a period of about 1 to about 20 minutes after dipping. In certain embodiments, the in vivo portion of the analyte sensor is dipped into the first polymer solution one or more times to generate a first polymer membrane having a thickness between about 5 μm to about 40 μm. In certain embodiments, the in vivo portion of the analyte sensor is dipped into the first polymer solution at least five times. In certain embodiments, the ratio of the thinnest point to the thickest point of the second polymer membrane is less than about 0.9.

In certain embodiments, the method for manufacturing an analyte sensor comprising a therapeutic agent comprises: providing an analyte sensor comprising an in vivo portion configured to reside below a skin surface of a subject and in contact with interstitial fluid of the subject, wherein the in vivo portion comprises a second region comprising an active area and a first region distal to the second region; preparing a first polymer solution comprising a first polymer or providing a first polymer solution comprising a first polymer; preparing a second polymer solution comprising a second polymer and a therapeutic agent or providing a second polymer solution comprising a second polymer and a therapeutic agent; contacting the in vivo portion with the first polymer solution to coat the first region and the second region to generate a first polymer membrane; and contacting the first region with the second polymer solution to coat the first region but not the second region to generate a second polymer membrane comprising the therapeutic agent.

In certain embodiments, contacting the in vivo portion with the first polymer solution comprises a screen printing, a rotary printing, a jetting, an aerosol deposition, a spray coating, a dip coating, a drop-casting, a spin coating or a brush coating process. In certain embodiments, contacting the first region with the second polymer solution comprises a screen printing, a rotary printing, a jetting, an aerosol deposition, a spray coating, a dip coating, a drop-casting, a spin coating or a brush coating process.

In certain embodiments, the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof. In certain embodiments, the anti-inflammatory agent is dexamethasone, a derivative thereof, or a salt form thereof.

In certain embodiments, the second polymer solution comprises the polymer, the therapeutic agent and a solvent. In certain embodiments, the solvent comprises an alcohol, a buffer or a combination thereof. In certain embodiments, the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof. In certain embodiments, the second polymer solution further comprises a crosslinker. In certain embodiments, the therapeutic agent is present in the second polymer solution in a range from about 0.01 wt % to about 50 wt % based on the total weight of the polymer solution. In certain embodiments, the polymer is present in the second polymer solution in a range from about 0.01 wt % to about 50 wt % based on the total weight of the polymer solution. In certain embodiments, the crosslinker is present in the second polymer solution in a range from about 0.01 wt % to about 50 wt % based on the total weight of the polymer solution. In certain embodiments, the second polymer solution has a viscosity of about 20 cP to about 250 cP. In certain embodiments, the second polymer solution has a viscosity of about 10 cP to about 150 cP. In certain embodiments, the second polymer solution has a viscosity of about 20 cP to about 80 cP.

In certain embodiments, the second polymer of the second polymer solution comprises is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof. In certain embodiments, the polyvinylpyridine-based polymer is a polyvinylpyridine-based copolymer. In certain embodiments, the polyvinylpyridine-based copolymer is a polyvinylpyridine-co-polystyrene polymer.

In certain embodiments, the first region is contacted with the second polymer solution for a time period of about 1 second to about 5 seconds. In certain embodiments, the method further comprises drying the second polymer solution for a period of about 1 to about 20 minutes after contact. In certain embodiments, the first region of the analyte sensor is contacted with the second polymer solution one or more times to generate a second polymer membrane having a thickness between about 5 μm to about 40 μm. In certain embodiments, the first region of the analyte sensor is contacted with the second polymer solution at least three times. In certain embodiments, the first region is ablated or planed prior to the contact of the first region with the second polymer solution. In certain embodiments, the second region has a length of about 5 μm to about 50 μm. In certain embodiments, the first region has a length of about 5 μm to about 50 μm. In certain embodiments, the first region has a length of about 5 μm to about 20 μm.

In certain embodiments, the active area comprises a plurality of sensing regions. In certain embodiments, at least one of the plurality of sensing regions is a spot.

In certain embodiments, contacting the in vivo portion with the first solution is performed before contacting the first region with the second polymer solution. In certain embodiments, the second polymer membrane is at least partially covering the first polymer membrane. In certain embodiments, contacting the first region with the second polymer solution is performed before contacting the in vivo portion with the first solution. In certain embodiments, the first polymer membrane is at least partially covering the second polymer membrane.

In certain embodiments, the ratio of the thinnest point to the thickest point of the second polymer membrane is less than about 0.9.

In certain embodiments, the total amount of the therapeutic agent in the second polymer membrane is from about 0.01 μg to about 500 μg. In certain embodiments, the total amount of the therapeutic agent in the second polymer membrane is less than about 5 μg.

In certain embodiments, a method for manufacturing an analyte sensor comprising a therapeutic agent comprises: providing an analyte sensor comprising (i) a working electrode, (ii) a counter and/or a reference electrode and (iii) an active area; preparing a first polymer solution comprising a first polymer or providing a first polymer solution comprising a first polymer; preparing a second polymer solution comprising a second polymer and a therapeutic agent or providing a second polymer solution comprising a second polymer and a therapeutic agent; depositing the second polymer solution on at least a portion of the counter and/or a reference electrode to generate a therapeutic agent-containing polymer composition; and depositing the first polymer solution on the in vivo portion to generate a first polymer membrane on top at least a portion of the therapeutic agent-containing polymer composition. In certain embodiments, depositing the second polymer solution on at least a portion of the counter and/or reference electrode comprises a screen printing, a rotary printing, a jetting, an aerosol deposition, a spray coating, a dip coating, a drop-casting, a spin coating or a brush coating process. In certain embodiments, depositing the first polymer solution on the in vivo portion comprises a screen printing, a rotary printing, a jetting, an aerosol deposition, a spray coating, a dip coating, a drop-casting, a spin coating or a brush coating process.

In certain embodiments, depositing the second polymer solution on the counter and/or reference electrode comprising depositing the second polymer solution onto the counter and/or reference electrode as two or more spots of the therapeutic agent-containing polymer composition. In certain embodiments, at least one of the spots has a circle shape, an oval shape, a regular polygon shape or an irregular polygon shape. In certain embodiments, at least two of the two or more spots comprise the same amount of the therapeutic agent. In certain embodiments, the at least two of the two or more spots comprise different amounts of the therapeutic agent. In certain embodiments, the difference in the amount of therapeutic agent between the at least two of the two or more spots is about 10% or more. In certain embodiments, at least two of the two or more spots comprise the same thickness. In certain embodiments, at least two of the two or more spots comprise different thicknesses. In certain embodiments, the difference in the thickness of two spots is about 10% or more.

The present disclosure further provides a method for fabricating an analyte sensor comprising a therapeutic agent, wherein the method comprises patterning a plurality of first conductive layers on a substrate to generate a plurality of working electrodes; patterning a plurality of second conductive layers on the substrate to generate a plurality of counter and/or reference electrodes; forming one or more spots of a therapeutic agent-containing polymer composition on each counter and/or reference electrode of the plurality of counter and/or reference electrodes, wherein the therapeutic agent-containing polymer composition comprises a polymer and a therapeutic agent; and singulating individual analyte sensors from the substrate, wherein each individual analyte sensor comprises at least one working electrode and at least one counter and/or reference electrode. In certain embodiments, the one or more spots has a circle shape, an oval shape, a regular polygon shape or an irregular polygon shape.

In certain embodiments, two or more spots are formed on the counter and/or reference electrode, wherein at least two of the two or more spots comprise the same amount of the therapeutic agent. In certain embodiments, two or more spots are formed on the counter and/or reference electrode, wherein at least two of the two spots comprise different amounts of the therapeutic agent. In certain embodiments, the difference in the amount of therapeutic agent between the two or more spots is about 10% or more. In certain embodiments, two or more spots are formed on the counter and/or reference electrode, wherein at least two of the two spots comprise the same thickness. In certain embodiments, two or more spots are formed on the counter and/or reference electrode, wherein at least two of the two spots comprise different thicknesses. In certain embodiments, the difference in the thickness of the two or more discrete area is about 10% or more.

In certain embodiments, forming the one or more spots of a therapeutic agent-containing polymer composition on each of the plurality of counter and/or reference electrodes comprises dispensing two or more layers of a polymer solution comprising the therapeutic agent. In certain embodiments, at least one of the two or more spots of a therapeutic agent-containing polymer composition is formed by dispensing two layers of the polymer solution and a second spot of the two or more spots of a therapeutic agent-containing polymer composition is formed by dispensing three layers of the polymer solution.

In certain embodiments, the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof. In certain embodiments, the anti-inflammatory agent is dexamethasone, a derivative thereof, or a salt form thereof.

In certain embodiments, the second polymer solution or the polymer solution comprises the polymer, the therapeutic agent and a solvent. In certain embodiments, the solvent comprises an alcohol, a buffer or a combination thereof. In certain embodiments, the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof. In certain embodiments, the second polymer solution or the polymer solution further comprises a crosslinker. In certain embodiments, the therapeutic agent is present in the second polymer solution or the polymer solution in a range from about 0.01 wt % to about 50 wt % based on the total weight of the polymer solution. In certain embodiments, the polymer is present in the second polymer solution or the polymer solution in a range from about 0.01 wt % to about 50 wt % based on the total weight of the polymer solution. In certain embodiments, the crosslinker is present in the second polymer solution or the polymer solution in a range from about 0.01 wt % to about 50 wt % based on the total weight of the polymer solution. In certain embodiments, the second polymer solution or the polymer solution has a viscosity of about 20 cP to about 250 cP. In certain embodiments, the second polymer solution or the polymer solution has a viscosity of about 20 cP to about 80 cP. In certain embodiments, the polymer of the second polymer solution or the polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof. In certain embodiments, the polyvinylpyridine-based polymer is a polyvinylpyridine-based copolymer. In certain embodiments, the polyvinylpyridine-based copolymer is a polyvinylpyridine-co-polystyrene polymer.

In certain embodiments, the method further comprises forming an active area on each of the plurality of working electrodes. In certain embodiments, the active area comprises a plurality of sensing regions. In certain embodiments, at least one of the plurality of sensing regions is a sensing spot. In certain embodiments, the method further comprises forming a membrane over at least a portion of the therapeutic agent-containing polymer composition of the individual analyte sensors. In certain embodiments, the membrane comprises a polymer selected from a group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.

The present disclosure further provides an analyte sensor. In certain embodiments, the analyte sensor comprises: a sensor tail comprising a non-sensing region and an active sensing region comprising a plurality of sensing spots, wherein the non-sensing region is around the active sensing region and comprises a drug-loading structure filled with a therapeutical agent-containing polymer composition. In certain embodiments, the drug-loading structure comprises at least one selected from a slot, a hole, a pore, a groove, and a depression. In certain embodiments, the drug-loading structure comprises two slots symmetrically arranged along the plurality of sensing spots. In certain embodiments, the drug-loading structure comprises a plurality of holes symmetrically arranged around the plurality of sensing spots. In certain embodiments, the slot is in a shape of an oval, a shape of a polygon, or an irregular shape. In certain embodiments, the hole is in a shape of a circle, a shape of a regular polygon, or a shape of an irregular polygon. In certain embodiments, the therapeutical agent-containing polymer composition comprises a polymer and a therapeutical agent. In certain embodiments, the polymer matrix comprises a copolymer selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof. In certain embodiments, the polyvinylpyridine-based copolymer is a polyvinylpyridine-co-polystyrene polymer or a derivative thereof. In certain embodiments, the polyvinylpyridine-co-polystyrene polymer can include 1-50 mer % of styrene units. In certain embodiments, the therapeutic agent is at least one selected from group consisting of an anti-inflammatory agent, an antiplatelet agent, an anticoagulant agent, a coagulant agent, an antiglycolytic agent, an antibiotic agent, an antiviral agent, and combinations thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof. In certain embodiments, the anti-inflammatory agent is dexamethasone, a derivative thereof, or a salt form thereof. In certain embodiments, the therapeutical agent is in a range of 0.01 wt %-50 wt % based on a total weight of the therapeutic agent-containing polymer composition. In certain embodiments, the analyte sensor is a subcutaneous sensor. In certain embodiments, the analyte sensor is configured to detect glucose.

The present disclosure further provides methods for preparing an analyte sensor that includes (i) providing a sensor tail comprising a non-sensing region and an active sensing region comprising a plurality of sensing spots; (ii) cutting at least a portion of the non-sensing region of the sensor tail to form a drug-loading structure; and (iii) filling the drug-loading structure with a therapeutic agent-containing polymer composition. In certain embodiments, the drug-loading structure comprises at least one selected from a slot, a hole, a pore, a groove, and a depression. In certain embodiments, the drug-loading structure comprises two slots symmetrically arranged along the plurality of sensing spots. In certain embodiments, the drug-loading structure comprises a plurality of holes symmetrically arranged around the plurality of sensing spots. In certain embodiments, the slot is in a shape of an oval, a shape of a polygon, or an irregular shape. In certain embodiments, the hole is in a shape of a circle, a shape of a regular polygon, or a shape of an irregular polygon. In certain embodiments, the therapeutical agent-containing polymer composition comprises a polymer and a therapeutical agent. In certain embodiments, the polymer comprises a copolymer selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof. In certain embodiments, the polyvinylpyridine-based copolymer is a polyvinylpyridine-co-polystyrene polymer or a derivative thereof. In certain embodiments, the polyvinylpyridine-co-polystyrene polymer can include 1-50 mer % of styrene units. In certain embodiments, the therapeutic agent is at least one selected from group consisting of an anti-inflammatory agent, an antiplatelet agent, an anticoagulant agent, a coagulant agent, an antiglycolytic agent, an antibiotic agent, an antiviral agent, and combinations thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof. In certain embodiments, the anti-inflammatory agent is dexamethasone, a derivative thereof, or a salt form thereof. In certain embodiments, the therapeutical agent is in a range of 0.01 wt %-50 wt % based on a total weight of the therapeutic agent-containing polymer composition.

The present disclosure further provides an insertion device. In certain embodiments, the insertion device includes an insertion tip configured to penetrate skin and coated with a therapeutic agent-containing polymer composition comprising a therapeutic agent and a polymer; and an analyte sensor comprising: a working electrode; an active area disposed upon the working electrode; and a membrane overcoating at least the active area. In certain embodiments, the insertion tip is retractable. In certain embodiments, the therapeutic agent-containing polymer composition overcoating the insertion tip is detachably attached to the insertion tip. In certain embodiments, the therapeutic agent-containing polymer composition is configured to remain in a tissue of a subject upon retraction of the sharp. In certain embodiments, the therapeutic agent-containing polymer composition is configured to release the therapeutic agent to an area surrounding an insertion site of the analyte sensor. In certain embodiments, the therapeutic agent is selected from the group consisting of an antibiotic agent, an antiviral agent, an anti-inflammatory agent, an anti-cancer agent, an antiplatelet agent, an anticoagulant agent, an antiglycolytic agent, and combinations thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and combinations thereof. In certain embodiments, the anti-inflammatory agent is dexamethasone. In certain embodiments, the polymer of the therapeutic agent-containing polymer composition is a bioresorbable polymer. In certain embodiments, the polymer is selected from the group consisting of a polyvinylpyridine-based polymer, a polyvinylimidazole-based polymer, a polyacrylate-based polymer, a polyurethane-based copolymer, a polyether urethane-based polymer, a silicone-based polymer, a polycaprolactone polymer, a polylactide polymer, a polyglycolide polymer, a polyethylene glycol polymer, a derivative thereof, and combinations thereof.

In certain embodiments, the therapeutic agent is covalently bound to the polymer.

In certain embodiments, the therapeutic agent is not bound to the polymer.

In certain embodiments, the membrane comprises poly(4-vinylpyridine).

In certain embodiments, the active area comprises one or more enzymes configured to detect an analyte. In certain embodiments, the analyte is selected from the group consisting of glutamate, glucose, ketones, lactate, oxygen, glycated hemoglobin (HbA1c), albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, and combinations thereof. In certain embodiments, the active area further comprises an electron transfer agent. In certain embodiments, the electron transfer agent comprises a transition metal complex. In certain embodiments, the analyte sensor further comprises an albumin and optionally a pH buffer.

In certain embodiments, the analyte sensor further comprises a reference electrode, a counter electrode, or both a reference electrode and a counter electrode. In certain embodiments, the analyte sensor further comprises a second active area that is responsive to a second analyte. In certain embodiments, the second analyte is glucose, lactate, or ketone. In certain embodiments, the therapeutic agent-containing polymer composition is configured to release the therapeutic agent at a predetermined release rate. In certain embodiments, the therapeutic agent-containing polymer composition can continuously release the therapeutic agent at a drug delivery rate of about 0.01 μg/day to about 1 mg/day of the therapeutic agent. In certain embodiments, the therapeutical agent is in a range of 0.01 wt %-50 wt % based on a total weight of the therapeutic agent-containing polymer composition and/or wherein the therapeutic agent-containing polymer composition about 0.01 μg to about 100 μg of the therapeutic agent. In certain embodiments, the therapeutic agent-containing polymer composition further comprises a crosslinker.

The present disclosure further provides a method of inhibiting an immune response at a sharp insertion site. In certain embodiments, the method comprises introducing into a tissue at the insertion site by use of a sharp, an analyte sensor comprising a therapeutic agent-containing polymer comprising a polymer and an effective amount of a therapeutic agent suitable for inhibiting an immune response at the insertion site, wherein the therapeutic agent-containing polymer composition coats the insertion tip; and maintaining the therapeutic agent-containing polymer composition in the tissue following retraction of the sharp. In certain embodiments, the therapeutic agent-containing polymer composition overcoating an insertion tip of the sharp is detachably attached to the insertion tip. In certain embodiments, the therapeutic agent is selected from the group consisting of an antibiotic agent, an antiviral agent, an anti-inflammatory agent, an anti-cancer agent, an antiplatelet agent, an anticoagulant agent, an antiglycolytic agent, and combinations thereof. In certain embodiments, the therapeutic agent is an anti-inflammatory agent. In certain embodiments, the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and combinations thereof. In certain embodiments, the anti-inflammatory agent is dexamethasone. In certain embodiments, the polymer of the therapeutic agent-containing polymer composition is a bioresorbable polymer. In certain embodiments, the polymer is selected from the group consisting of a polyvinylpyridine-based polymer, a polyvinylimidazole-based polymer, a polyacrylate-based polymer, a polyurethane-based copolymer, a polyether urethane-based polymer, a silicone-based polymer, a polycaprolactone polymer, a polylactide polymer, a polyglycolide polymer, a polyethylene glycol polymer, a derivative thereof, and combinations thereof. In certain embodiments, the therapeutic agent is covalently bound to the polymer composition. In certain embodiments, the therapeutic agent is not bound to the polymer composition.

In certain embodiments, the analyte sensor comprises a working electrode, an active area disposed on the working electrode, and a membrane overcoating at least the active area, wherein the analyte sensor is separated from the therapeutic agent. In certain embodiments, the membrane comprises poly(4-vinylpyridine). In certain embodiments, the active area comprises one or more enzymes configured to detect an analyte. In certain embodiments, the analyte is selected from the group consisting of glutamate, glucose, ketones, lactate, oxygen, glycated hemoglobin (HbA1c), albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, and combinations thereof. In certain embodiments, the analyte is glucose, lactate, or ketone. In certain embodiments, the active area further comprises an electron transfer agent. In certain embodiments, the electron transfer agent comprises a transition metal complex. In certain embodiments, the active area further comprises an albumin and optionally a pH buffer. In certain embodiments, the analyte sensor further comprises a reference electrode, a counter electrode, or both a reference electrode and a counter electrode. In certain embodiments, the therapeutic agent-containing polymer composition is configured to release the active agent to an area surrounding an insertion site of the sensor.

In certain embodiments, the therapeutic agent-containing polymer composition is configured to release the therapeutic agent at a predetermined release rate. In certain embodiments, the therapeutic agent-containing polymer composition can continuously release the therapeutic agent at a drug delivery rate of about 0.01 μg/day to about 1 mg/day of the therapeutic agent. In certain embodiments, the therapeutic agent-containing polymer composition can continuously release the therapeutic agent at a drug delivery rate of about 0.001 μg/day to about 1 μg/day of the therapeutic agent. In certain embodiments, the therapeutical agent is in a range of 0.01 wt %-50 wt % based on a total weight of the therapeutic agent-containing polymer composition and/or wherein the therapeutic agent-containing polymer composition about 0.01 μg to about 100 μg of the therapeutic agent. In certain embodiments, the therapeutic agent-containing polymer composition further comprises a crosslinker.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1A illustrates a cross-sectional diagram of an exemplary analyte sensor according to one or more embodiments of the present disclosure.

FIG. 1B illustrates a cross-sectional diagram of an exemplary analyte sensor according to one or more embodiments of the present disclosure.

FIG. 2 illustrates a cross-sectional diagram of an exemplary analyte sensor according to one or more embodiments of the present disclosure.

FIGS. 3A-3D illustrate schematic plan views of exemplary sensor tails comprising drug-loading structures according to one or more embodiments of the present disclosure.

FIG. 4 illustrates a schematic view of an exemplary analyte sensor according to one or more embodiments of the present disclosure.

FIGS. 5A-5C illustrate schematic views of an exemplary analyte sensor comprising drug-loading structures according to one or more embodiments of the present disclosure.

FIG. 6 illustrates a flow chart of a method for preparing an exemplary analyte sensor according to one or more embodiments of the present disclosure.

FIG. 7 illustrates a schematic view of an exemplary sensor tail according to one or more embodiments of the present disclosure.

FIG. 8 illustrates exemplary steps, tasks, or acts of a method for coating an analyte sensor according to one or more embodiments of the present disclosure.

FIG. 9 illustrates a schematic view of an exemplary coated analyte sensor according to one or more embodiments of the present disclosure.

FIG. 10 illustrates an exemplary sensor tail including a mass-limiting membrane and a drug-eluting membrane according to one or more embodiments of the present disclosure.

FIG. 11 illustrates an exemplary sensor tail including a mass-limiting membrane and a drug-eluting membrane according to one or more embodiments of the present disclosure.

FIG. 12A illustrates an exemplary sensor tail including two dexamethasone containing spots on the counter electrode according to one or more embodiments of the present disclosure.

FIG. 12B illustrates a schematic view of an exemplary coated analyte sensor having two dexamethasone containing spots on the counter electrode according to one or more embodiments of the present disclosure and the exemplary thicknesses for each of the spots.

FIG. 13A illustrates exemplary analyte sensor tails that include a mass-limiting membrane and a drug-eluting membrane disposed upon the mass-limiting membrane according to one or more embodiments of the present disclosure.

FIG. 13B illustrates exemplary analyte sensor tails that include a mass-limiting membrane and a drug-eluting membrane disposed upon the mass-limiting membrane according to one or more embodiments of the present disclosure.

FIG. 14A illustrates exemplary analyte sensor tails that include a mass-limiting membrane and a drug-eluting membrane located beneath the mass-limiting membrane according to one or more embodiments of the present disclosure.

FIG. 14B illustrates exemplary analyte sensor tails that include a mass-limiting membrane and a drug-eluting membrane located beneath the mass-limiting membrane according to one or more embodiments of the present disclosure.

FIG. 14C illustrates an exemplary sensor tail including a drug-eluting membrane located on the sensor tail according to one or more embodiments of the present disclosure.

FIG. 15 illustrates exemplary steps, tasks, or acts of a method for coating an analyte sensor according to one or more embodiments of the present disclosure.

FIG. 16 provides a diagram of an illustrative sensing system that can incorporate an analyte sensor of the present disclosure.

FIGS. 17A-17B provide cross-sectional diagrams of exemplary analyte sensors including a single active area.

FIGS. 18A-18C provide cross-sectional diagrams of exemplary analyte sensors including two active areas upon separate working electrodes.

FIG. 19 provides a cross-sectional diagram of an exemplary analyte sensor including two active areas.

FIGS. 20A-20C provide perspective view of exemplary analyte sensors including two active areas upon separate working electrodes.

FIG. 21 provides the drug delivery profiles of exemplary drug-eluting membranes disposed upon or under a mass-limiting membrane on an analyte sensor according to certain embodiments of the present disclosure.

FIG. 22 provides the thickness profiles of exemplary drug-eluting membranes disposed upon or under a mass-limiting membrane on an analyte sensor according to one or more embodiments of the present disclosure.

FIGS. 23A-23B provide exemplary schematics of a sharp coated with a therapeutic agent-containing polymer composition. FIG. 23A provides an exemplary front view of the moment when a sensor tail is inserted into skin with a sharp coated in a therapeutic agent-containing polymer composition loaded with a therapeutic agent. FIG. 23B provides an exemplary schematic showing that after insertion, the sharp is retracted back to the applicator and the therapeutic agent-containing polymer composition is retained in the skin in the vicinity of the sensor.

FIGS. 24A-24B provide exemplary schematics of top-view cross-sections of a sharp coated with a therapeutic agent-containing polymer composition. FIG. 24A provides an exemplary schematic of a top-view cross-section of the moment when an analyte sensor tail is inserted into the skin with the help of a sharp coated in a therapeutic agent-containing polymer composition loaded with a therapeutic agent. FIG. 24B provides an exemplary schematic showing that after insertion, the sharp is retracted back to the applicator and the therapeutic agent-containing polymer composition is retained in the skin in the vicinity of the sensor.

### DETAILED DESCRIPTION

As described herein, the implantation of an analyte sensor can result in several physiological responses that can negatively impact sensor function. For example, inflammation or immune responses at sites of tissue trauma induced by the analyte sensor and its implantation can result in a loss of sensor functionality and sensitivity in vivo.

To address the foregoing needs, the present disclosure provides analyte sensors and sharps that include a therapeutic agent. The present disclosure further provides methods for depositing a therapeutic agent, and compositions thereof, onto an analyte sensor and a sharp coated with a therapeutic agent for introducing an analyte sensor into a subject.

In certain embodiments, the sustained release of a therapeutic agent, e.g., an anti-inflammatory agent, in close proximity to an analyte sensor can result in the prevention and/or reduction of inflammation or immune responses in the tissue surrounding the implantation site. For example, but not by way of limitation, the prevention and/or reduction of inflammation in the tissue surrounding the implantation site can increase the life span of the implanted analyte sensor. In certain embodiments, preventing and/or reducing the immune response to the analyte sensor can increase the life span of the implanted analyte sensor. In certain embodiments, increasing the life span of the implanted analyte sensor refers to maintaining the accuracy of the analyte sensor towards the end of the life of the sensor and/or minimizing, reducing and/or eliminating analyte signal inaccuracies towards the end of the life of the sensor.

In certain embodiments, the life span of an analyte sensor disclosed herein can be increased by more than about 2 days, by more than about 3 days, by more than about 4 days, by more than about 5 days, by more than about 6 days, by more than about 7 days, by more than about 8 days, by more than about 9 days, by more than about 10 days, by more than about 11 days, by more than about 12 days, by more than about 13 days, by more than about 14 days, by more than about 15 days, by more than about 16 days, by more than about 17 days, by more than about 18 days, by more than about 19 days or by more than about 20 days. In certain embodiments, an analyte sensor that includes a therapeutic agent-containing polymer composition of the present disclosure can have a life span of about 14 days or more, about 15 days or more, about 16 days or more, about 17 days or more, about 18 days or more, about 19 days or more, about 20 days or more, about 21 days or more, about 22 days or more, about 23 days or more, about 24 days or more, about 25 days or more, about 26 days or more, about 27 days or more, about 28 days or more, about 29 days or more or about 30 days or more. In certain embodiments, the life span of an analyte sensor disclosed herein can be increased to obtain an analyte sensor that has a life span of about 30 days or greater.

Hereinafter, specific embodiments will be described in more detail so that those of ordinary skill in the art can easily implement them. For example, embodiments of the present disclosure will be explained in more detail referring to the drawings. However, this disclosure can be embodied in many different forms and is not construed as limited to the example embodiments set forth herein.

For clarity, but not by way of limitation, the detailed description of the presently disclosed subject matter is divided into the following subsections:
I. Definitions;
II. Therapeutic Agents;
III. Analyte Sensors;
IV. Therapeutic Agent-Containing Polymer Compositions;
V. Incorporation of Therapeutic Agents into Analyte Sensors;
VI. Methods of Manufacture;
VII. Insertion Devices; and
VIII. Exemplary Embodiments

### I. Definitions

The terms utilized in the present disclosure generally have their ordinary meanings in the art, within the context of this disclosure and in the specific context where each term is utilized. Like reference numerals can refer to like elements throughout the present disclosure. Certain terms are discussed below, or elsewhere in the specification, to provide additional guidance to the skilled artisan in describing the compositions and methods of the present disclosure and how to make and utilize them.

The terminology utilized herein is utilized to describe embodiments only and is not intended to limit the present disclosure. In the present disclosure, although the terms “first,” “second,” etc., can be utilized herein to describe one or more elements, components, regions, and/or layers, these elements, components, regions, and/or layers should not be limited by these terms. These terms are only utilized to distinguish one component from another component.

As utilized herein, the singular forms “a,” “an,” and “the” are intended to include the plural forms as well, unless the context clearly indicates otherwise. Further, the utilization of “can” when describing embodiments of the present disclosure refers to “one or more embodiments of the present disclosure.”

The terms “comprise(s),” “include(s),” “having,” “has,” “contain(s),” and variants thereof, as utilized herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

Herein, “or” is not to be construed as an exclusive meaning, for example, “A or B” is construed to include A, B, A+B, and/or the like. Further, as utilized herein, the term “and/or” includes any and all combinations of one or more of the associated listed items. Expressions such as “at least one of,” “one of,” and “selected from,” when preceding a list of elements, modify the entire list of elements and do not modify the individual elements of the list.

The term “about” or “approximately” can refer to within an acceptable error range for the particular value as determined by one of ordinary skill in the art, which depends in part on how the value is measured or determined, i.e., the limitations of the measurement system. For example, “about” can refer to within 3 or more than 3 standard deviations, per the practice in the art. Alternatively, “about” can refer to a range of up to 20%, up to 10%, up to 5%, or up to 1% of a given value. Alternatively, particularly with respect to biological systems or processes, the term can refer to within an order of magnitude, for example, within 5-fold, or within 2-fold, of a value.

As utilized herein, “analyte sensor” or “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glutamate, glucose, ketones, lactate, oxygen, hemoglobin AlC, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, etc.

The term “biological fluid,” as used herein, refers to any bodily fluid or bodily fluid derivative in which the analyte can be measured. Non-limiting examples of a biological fluid include dermal fluid, interstitial fluid, plasma, blood, lymph, synovial fluid, cerebrospinal fluid, saliva, bronchoalveolar lavage, amniotic fluid, sweat, tears or the like. In certain embodiments, the biological fluid is dermal fluid or interstitial fluid. In certain embodiments, the biological fluid is interstitial fluid.

The term “covalent bond,” as utilized herein, refers to a chemical bond that involves the sharing of electron pairs between atoms. Likewise, “covalently bound” refers to chemical binding in a way that involves the sharing of electron pairs between atoms.

As used herein, the term “multi-component membrane” refers to a membrane comprising two or more types of membrane polymers.

The term “non-covalent,” or the similar, as utilized herein, refers to a chemical interaction that does not involve the sharing of electrons, but rather involves more dispersed variations of electromagnetic interactions between molecules or within a molecule.

As utilized herein, the term “polyvinylpyridine-based polymer” can refer to a polymer (e.g., a copolymer) that includes polyvinylpyridine (e.g., poly(2-vinylpyridine) or poly(4-vinylpyridine)) or a derivative thereof.

The term “reference electrode” as used herein, can refer to either reference electrodes or electrodes that function as both, a reference and a counter electrode. Similarly, the term “counter electrode,” as used herein, refers to both, a counter electrode and a counter electrode that also functions as a reference electrode. In certain embodiments, the term “counter/reference electrode,” as used herein, refers to both, a counter electrode and a counter electrode that also functions as a reference electrode.

As used herein, the term “single-component membrane” refers to a membrane comprising one type of membrane polymer.

### II. Therapeutic Agents

The present disclosure provides analyte sensors that include a therapeutic agent. In certain embodiments, an analyte sensor of the present disclosure can include two or more therapeutic agents.

The present disclosure further provides sharps that are coated with a therapeutic agent. In certain embodiments, a sharp of the present disclosure can be coated with two or more therapeutic agents.

In certain embodiments, the therapeutic agent to be delivered according to the present disclosure can be a therapeutic agent that is effective at reducing, minimizing, preventing and/or inhibiting a tissue's response to analyte sensor implantation. For example, but not by way of limitation, the therapeutic agent to be delivered according to the present disclosure can be a therapeutic agent that is effective as reducing, minimizing, preventing and/or inhibiting inflammation in a tissue. In certain embodiments, the therapeutic agent to be delivered according to the present disclosure can be a therapeutic agent that is effective at maintaining the accuracy of the analyte sensor towards the end of the life of the sensor and/or minimizing, reducing and/or eliminating analyte signal inaccuracies towards the end of the life of the sensor.

In certain embodiments, a therapeutic agent for use in the present disclosure can include at least one selected from group consisting of an antibiotic agent, an antiviral agent, an anti-inflammatory agent, an anti-cancer agent, an antiplatelet agent, an anticoagulant agent, a coagulant agent, an antiglycolytic agent and combinations thereof. In certain embodiments, the therapeutic agent is an antibiotic agent. In certain embodiments, the therapeutic agent is an antiviral agent. In certain embodiments, the therapeutic agent is an anti-inflammatory agent. In certain embodiments, the therapeutic agent is an anti-cancer agent. In certain embodiments, the therapeutic agent is an antiplatelet agent. In certain embodiments, the therapeutic agent is an anticoagulant agent. In certain embodiments, the therapeutic agent is a coagulant agent. In certain embodiments, the therapeutic agent is an antiglycolytic agent.

In certain embodiments, the therapeutic agent is an antiviral agent. In certain embodiments, the antiviral agent can include, but is not limited to, Umifenovir, Baloxavir marboxil, Darunavir, Nitazoxanide, Peramivir, Tipranavir, etc.

In certain embodiments, the therapeutic agent is an antibiotic agent. In certain embodiments, the antibiotic agent can include, but is not limited to, Rifaximin, Ertapenem, Doripenem, Cefadroxil, Clindamycin, Amoxicillin, Penicillin, etc.

In certain embodiments, the therapeutic agent is an anti-cancer agent. In certain embodiments, the anti-cancer agent can include, but is not limited to, Gilteritinib, Glasdegib, Ivosidenib, Enasidenib, Midostaurin, Venetoclax, Alpelisib, etc.

In certain embodiments, a therapeutic agent for use in the present disclosure can be an immunosuppressant. Non-limiting examples of immunosuppressants include anti-inflammatory agents, anti-cancer agents, anti-rejection drugs and combinations thereof.

In certain embodiments, the therapeutic agent can be an anti-inflammatory agent. In certain embodiments, the anti-inflammatory agent is a non-steroidal anti-inflammatory agent. In certain embodiments, the anti-inflammatory agent is a steroidal anti-inflammatory agent, e.g., a corticosteroid. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. Non-limiting salt forms include pharmaceutically acceptable salts including acetate and phosphate salts. In certain embodiments, the anti-inflammatory agent is a salt of dexamethasone.

In certain embodiments, the anti-inflammatory agent is dexamethasone.

In certain embodiments, the anti-inflammatory agent is dexamethasone or a derivative or a salt form thereof.

In certain embodiments, the derivative and/or salt form of dexamethasone is dexamethasone acetate.

In certain embodiments, the derivative and/or salt form of dexamethasone is dexamethasone sodium phosphate.

### III. Analyte Sensors

### A. General Structure of Analyte Sensor Systems

The present disclosure relates to the incorporation of therapeutic agents into an analyte sensor, e.g., an in vivo analyte sensor, and/or the delivery of a therapeutic agent in close proximity to an analyte sensor (e.g., by use of a sharp coated with a therapeutic agent).

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood analyte level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

FIG. 16 provides a diagram of an illustrative sensing system that can incorporate an analyte sensor of the present disclosure. As shown, sensing system 100 includes sensor control device 102 and reader device 120 that are configured to communicate with one another over a local communication path or link 140, which can be wired or wireless, uni- or bi-directional, and encrypted or non-encrypted. Reader device 120 can constitute an output medium for viewing analyte concentrations and alerts or notifications determined by sensor 104 or a processor associated therewith, as well as allowing for one or more user inputs, according to certain embodiments. Reader device 120 can be a multi-purpose smartphone or a dedicated electronic reader instrument. While only one reader device 120 is shown, multiple reader devices 120 can be present in certain instances. Reader device 120 can also be in communication with remote terminal 170 and/or trusted computer system 180 via communication path(s)/link(s) 141 and/or 142, respectively, which also can be wired or wireless, uni- or bi-directional, and encrypted or non-encrypted. Reader device 120 can also or alternately be in communication with network 150 (e.g., a mobile telephone network, the internet, or a cloud server) via communication path/link 151. Network 150 can be further communicatively coupled to remote terminal 170 via communication path/link 152 and/or trusted computer system 180 via communication path/link 153. Alternately, sensor 104 can communicate directly with remote terminal 170 and/or trusted computer system 180 without an intervening reader device 120 being present. For example, but not by the way of limitation, sensor 104 can communicate with remote terminal 170 and/or trusted computer system 180 through a direct communication link to network 150, according to certain embodiments, as described in U.S. Patent Application Publication 2011/0213225 and incorporated herein by reference in its entirety. Any suitable electronic communication protocol can be used for each of the communication paths or links, such as near field communication (NFC), radio frequency identification (RFID), BLUETOOTH® or BLUETOOTH® Low Energy protocols, WiFi, or the like. Remote terminal 170 and/or trusted computer system 180 can be accessible, according to certain embodiments, by individuals other than a primary user who have an interest in the user's analyte levels. Reader device 120 can include display 122 and optional input component 121. Display 122 can include a touch-screen interface, according to certain embodiments.

Sensor control device 102 includes sensor housing 103, which can house circuitry and a power source for operating sensor 104. Optionally, the power source and/or active circuitry can be omitted. A processor (not shown) can be communicatively coupled to sensor 104, with the processor being physically located within sensor housing 103 or reader device 120. Sensor 104 protrudes from the underside of sensor housing 103 and extends through adhesive layer 105, which is adapted for adhering sensor housing 103 to a tissue surface, such as skin, according to certain embodiments.

B. In vivo Portion of Analyte Sensor

Sensor 104 of FIG. 16 is adapted to be at least partially inserted into a tissue of interest, such as within the dermal or subcutaneous layer of the skin. Sensor 104 can include an in vivo portion (also referred to herein as a sensor tail) of sufficient length for insertion to a desired depth in a given tissue. The in vivo portion is configured to reside below a skin surface of a subject and in contact with a fluid (e.g., interstitial fluid) of the subject. The in vivo portion can include at least one working electrode. In certain embodiments, the in vivo portion can include two working electrodes. In certain configurations, the in vivo portion can include an active area for detecting an analyte, e.g., on a working electrode. A counter electrode can be present in combination with the at least one working electrode. Particular electrode configurations upon the in vivo portion are described in more detail below.

The active area can be configured for detecting a particular analyte as described in further detail below. For example, but not by way of limitation, the analyte can be glucose, ketones, lactate, alcohol, glutamate, creatine, creatinine, sarcosine, ascorbate and a combination thereof. Additional non-limiting examples of analytes that can be detected by analyte sensors of the present disclosure are disclosed in Section III.B.ii herein. For example, but not by the way of limitation, a glucose-responsive active area can include a glucose-responsive enzyme, a ketones-responsive active area can include a ketones-responsive enzyme, a lactate-responsive active area can include a lactate-responsive enzyme, an alcohol-responsive active area can include an alcohol-responsive enzyme, a glutamate-responsive active area can include a glutamate-responsive enzyme, a creatine-responsive active area can include a creatine-responsive enzyme system, a creatinine-responsive active area can include a creatine-responsive enzyme system, a sarcosine-responsive active area can include a sarcosine-responsive enzyme system, and an ascorbate-responsive active area can include an ascorbate-responsive enzyme system.

A membrane can overcoat the active area, as also described in further detail below. In certain embodiments, a membrane overcoating an analyte-responsive active area can function as a mass transport limiting membrane and/or to improve biocompatibility. A mass transport limiting membrane can act as a diffusion-limiting barrier to reduce the rate of mass transport of the analyte. For example, but not by way of limitation, limiting access of an analyte to the analyte-responsive active area with a mass transport limiting membrane can aid in avoiding sensor overload (saturation), thereby improving detection performance and accuracy.

In certain embodiments of the present disclosure, one or more analytes can be monitored in any biological fluid of interest such as dermal fluid, interstitial fluid, plasma, blood, lymph, synovial fluid, cerebrospinal fluid, saliva, bronchoalveolar lavage, amniotic fluid, or the like. In certain embodiments, analyte sensors of the present disclosure can be adapted for assaying dermal fluid or interstitial fluid to determine a concentration of one or more analytes in vivo. In certain embodiments, the biological fluid is interstitial fluid.

Referring still to FIG. 16, sensor 104 can automatically forward data to reader device 120. For example, but not by the way of limitation, analyte concentration data can be communicated automatically and periodically, such as at a certain frequency as data is obtained or after a certain time period has passed, with the data being stored in a memory until transmittal (e.g., every minute, five minutes, or other predetermined time period). In certain embodiments, sensor 104 can communicate with reader device 120 in a non-automatic manner and not according to a set schedule. For example, but not by the way of limitation, data can be communicated from sensor 104 using RFID technology when the sensor electronics are brought into communication range of reader device 120. Until communicated to reader device 120, data can remain stored in a memory of sensor 104. Thus, a user does not have to maintain close proximity to reader device 120 at all times, and can instead upload data at a convenient time. In certain embodiments, a combination of automatic and non-automatic data transfer can be implemented. For example, and not by the way of limitation, data transfer can continue on an automatic basis until reader device 120 is no longer in communication range of sensor 104.

An introducer can be present transiently to promote introduction of sensor 104 into a tissue. In certain illustrative embodiments, the introducer can include a needle or similar sharp. As would be readily recognized by a person skilled in the art, other types of introducers, such as sheaths or blades, can be present in alternative embodiments. More specifically, the needle or other introducer can transiently reside in proximity to sensor 104 prior to tissue insertion and then be withdrawn afterward. While present, the needle or other introducer can facilitate insertion of sensor 104 into a tissue by opening an access pathway for sensor 104 to follow. For example, and not by the way of limitation, the needle can facilitate penetration of the epidermis as an access pathway to the dermis to allow implantation of sensor 104 to take place, according to one or more embodiments. After opening the access pathway, the needle or other introducer can be withdrawn so that it does not represent a sharps hazard. In certain embodiments, suitable needles can be solid or hollow, beveled or non-beveled, and/or circular or non-circular in cross-section. In more particular embodiments, suitable needles can be comparable in cross-sectional diameter and/or tip design to an acupuncture needle, which can have a cross-sectional diameter of about 250 microns. However, suitable needles can have a larger or smaller cross-sectional diameter if needed for certain particular applications. As disclosed herein, the needle or introducer can be at least partially coated with a therapeutic agent.

In certain embodiments, a tip of the needle (while present) can be angled over the terminus of sensor 104, such that the needle penetrates a tissue first and opens an access pathway for sensor 104. In certain embodiments, sensor 104 can reside within a lumen or groove of the needle, with the needle similarly opening an access pathway for sensor 104. In either case, the needle is subsequently withdrawn after facilitating sensor insertion.

i. Electrode Configuration

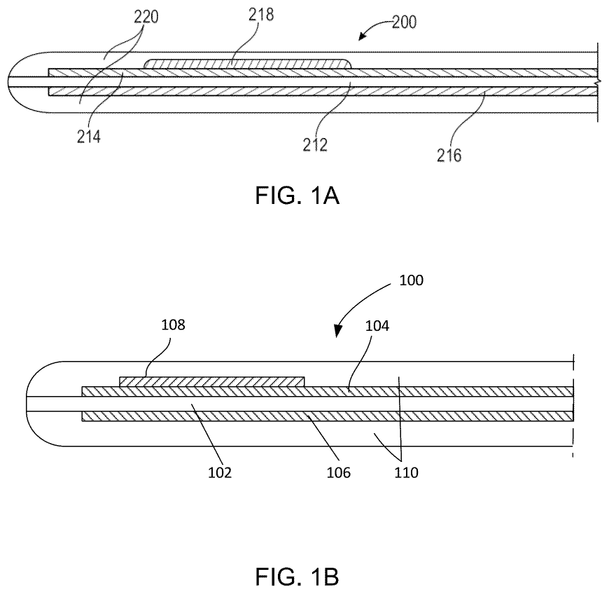

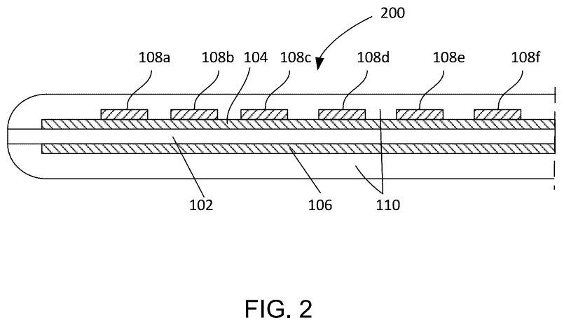

Sensor configurations featuring a single active area that is configured for detection of a corresponding single analyte can employ two-electrode or three-electrode detection motifs, as described further herein in reference to FIGS. 1A-1B, 2 and 17A-17B. Sensor configurations featuring two different active areas for detection of the same or separate analytes, either upon separate working electrodes or upon the same working electrode, are described separately thereafter in reference to FIGS. 2 and 18A-20C. Sensor configurations having multiple working electrodes can be particularly advantageous for incorporating two different active areas within the same in vivo portion (e.g., sensor tail), since the signal contribution from each active area can be determined more readily.

When a single working electrode is present in an analyte sensor, three-electrode sensor configurations can include a working electrode, a counter electrode and a reference electrode. Related two-electrode sensor configurations can include a working electrode and a second electrode, in which the second electrode can function as both a counter electrode and a reference electrode (i.e., a counter/reference electrode). The various electrodes can be at least partially stacked (layered) upon one another and/or laterally spaced apart from one another upon the sensor tail. Suitable sensor configurations can be substantially flat in shape, substantially cylindrical in shape or any suitable shape. In any of the sensor configurations disclosed herein, the various electrodes can be electrically isolated from one another by a dielectric material or similar insulator.

Analyte sensors featuring multiple working electrodes can similarly include at least one additional electrode. When one additional electrode is present, the one additional electrode can function as a counter/reference electrode for each of the multiple working electrodes. When two additional electrodes are present, one of the additional electrodes can function as a counter electrode for each of the multiple working electrodes and the other of the additional electrodes can function as a reference electrode for each of the multiple working electrodes.

FIG. TA shows a diagram of an illustrative two-electrode analyte sensor configuration, which is compatible for use in the disclosure herein. As shown, analyte sensor 200 includes substrate 212 disposed between working electrode 214 and counter/reference electrode 216. Alternately, working electrode 214 and counter/reference electrode 216 can be located upon the same side of substrate 212 with a dielectric material interposed in between (configuration not shown). Active area 218 is disposed as at least one layer upon at least a portion of working electrode 214. Active area 218 can include multiple spots or a single spot configured for detection of an analyte at a low working electrode potential, as discussed further herein. In certain embodiments, active area 218 can comprise an electron transfer agent described herein.

Referring still to FIG. TA, membrane 220 coats at least active area 218. In certain embodiments, membrane 220 comprises a copolymer of the present disclosure. For example, but not by way of limitation, membrane 220 comprises a copolymer comprising a first monomer, e.g., a styrene, and a second monomer comprising a heterocycle-containing component, e.g., a vinylpyridine, e.g., 4-vinylpyridine.

In certain embodiments, membrane 220 can also overcoat some or all of working electrode 214 and/or counter/reference electrode 216, or the entirety of analyte sensor 200. One or both faces of analyte sensor 200 can be coated with membrane 220. Membrane 220 can include one or more polymeric membrane materials having capabilities of limiting analyte flux to active area 218 (i.e., membrane 220 is a mass transport limiting membrane having some permeability for the analyte of interest). The composition and thickness of membrane 220 can vary to promote a desired analyte flux to active area 218, thereby providing a desired signal intensity and stability. Analyte sensor 200 can be operable for assaying an analyte by any of coulometric, amperometric, voltammetric, or potentiometric electrochemical detection techniques.

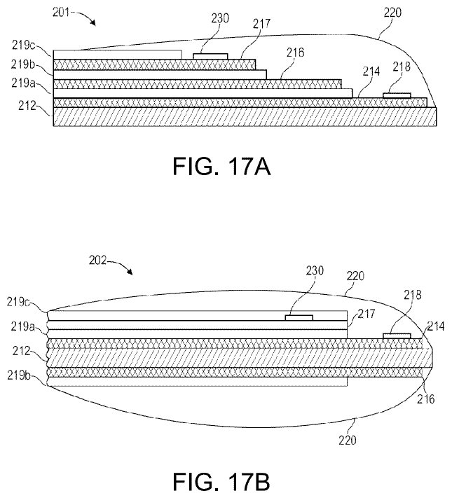

FIGS. 17A and 17B show diagrams of illustrative three-electrode analyte sensor configurations, which are also compatible for use in the disclosure herein. Three-electrode analyte sensor configurations can be similar to that shown for analyte sensor 200 in FIG. 1A, except for the inclusion of additional electrode 217 in analyte sensors 201 and 202 (FIGS. 17A and 17B). With additional electrode 217, counter/reference electrode 216 can then function as either a counter electrode or a reference electrode, and additional electrode 217 fulfills the other electrode function not otherwise accounted for. Working electrode 214 continues to fulfill its original function. Additional electrode 217 can be disposed upon either working electrode 214 or electrode 216, with a separating layer of dielectric material in between. For example, and not by the way of limitation, as depicted in FIG. 17A, dielectric layers 219a, 219b and 219c separate electrodes 214, 216 and 217 from one another and provide electrical isolation. Alternatively, at least one of electrodes 214, 216 and 217 can be located upon opposite faces of substrate 212, as shown in FIG. 17B. Thus, in certain embodiments, electrode 214 (working electrode) and electrode 216 (counter electrode) can be located upon opposite faces of substrate 212, with electrode 217 (reference electrode) being located upon one of electrodes 214 or 216 and spaced apart therefrom with a dielectric material. Reference material layer 230 (e.g., Ag/AgCl) can be present upon electrode 217, with the location of reference material layer 230 not being limited to that depicted in FIGS. 17A and 17B. As with sensor 200 shown in FIG. 1A, active area 218 in analyte sensors 201 and 202 can include multiple spots or a single spot. In certain embodiments, active area 218 can include a redox mediator disclosed herein. Additionally, analyte sensors 201 and 202 can be operable for assaying an analyte by any of coulometric, amperometric, voltammetric, or potentiometric electrochemical detection techniques.

Like analyte sensor 200, membrane 220 can also overcoat active area 218, as well as other sensor components, in analyte sensors 201 and 202, thereby serving as a mass transport limiting membrane. In certain embodiments, the additional electrode 217 can be coated with membrane 220. Although FIGS. 17A and 17B have depicted electrodes 214, 216 and 217 as being coated with membrane 220, it is to be recognized that in certain embodiments only working electrode 214 is coated. Moreover, the thickness of membrane 220 at each of electrodes 214, 216 and 217 can be the same or different. As in two-electrode analyte sensor configurations (FIG. 1A), one or both faces of analyte sensors 201 and 202 can be coated with membrane 220 in the sensor configurations of FIGS. 17A and 17B, or the entirety of analyte sensors 201 and 202 can be coated. Accordingly, the three-electrode sensor configurations shown in FIGS. 17A and 17B should be understood as being non-limiting of the embodiments disclosed herein, with alternative electrode and/or layer configurations remaining within the scope of the present disclosure.

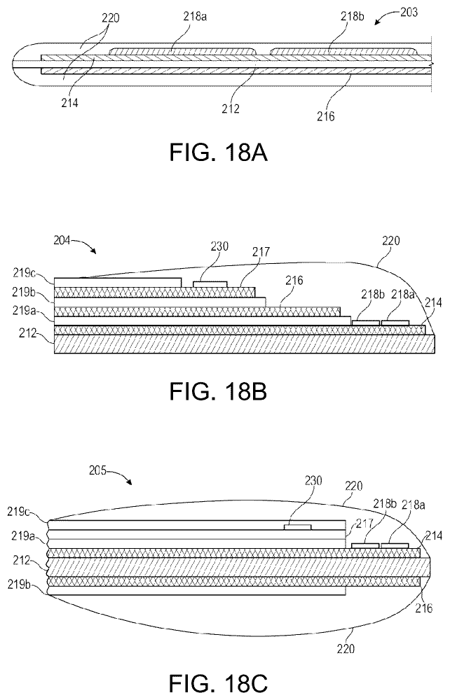

FIG. 18A shows an illustrative configuration for sensor 203 having a single working electrode with two different active areas disposed thereon. FIG. 18A is similar to FIG. 1A, except for the presence of two active areas upon working electrode 214: first active area 218a and second active area 218b, which are responsive to the same or different analytes and are laterally spaced apart from one another upon the surface of working electrode 214. Active areas 218a and 218b can include multiple spots or a single spot configured for detection of each analyte. The composition of membrane 220 can vary or be compositionally the same at active areas 218a and 218b. First active area 218a and second active area 218b can be configured to detect their corresponding analytes at working electrode potentials that differ from one another, as discussed further below.

FIGS. 18B and 18C show cross-sectional diagrams of illustrative three-electrode sensor configurations for sensors 204 and 205, respectively, each featuring a single working electrode having first active area 218a and second active area 218b disposed thereon. FIGS. 18B and 18C are otherwise similar to FIGS. 17A and 17B and can be better understood by reference thereto. As with FIG. 18A, the composition of membrane 220 can vary or be compositionally the same at active areas 218a and 218b. In certain embodiments, any one of active areas 218a and 218b can comprise a redox mediator described herein. In certain embodiments, only one of active areas 218a and 218b can comprise a redox mediator described herein. For example, but not by way of limitation, only active area 218a includes a redox mediator described herein. In certain embodiments, only active area 218b includes a redox mediator described herein. In certain embodiments, both active areas 218a and 218b comprise a redox mediator described herein. In certain embodiments, the electron transfer agent present in active area 218a is different from the redox mediator present in 218b. Alternatively, the electron transfer agent present in active area 218a is the same redox mediator present in 218b.

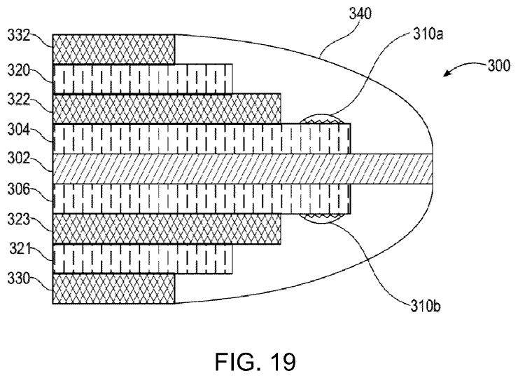

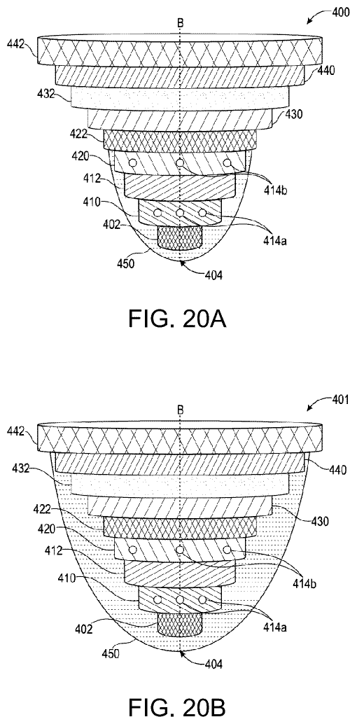

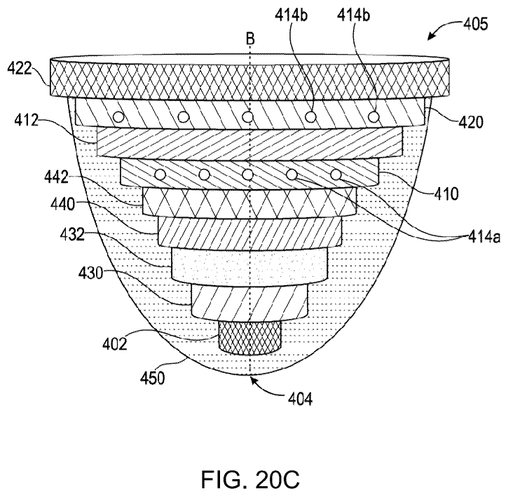

Illustrative sensor configurations having multiple working electrodes, specifically two working electrodes, are described in further detail in reference to FIGS. 19-20C. Although the following description is primarily directed to sensor configurations having two working electrodes, it is to be appreciated that more than two working electrodes can be incorporated through extension of the disclosure herein. Additional working electrodes can be used to impart additional sensing capabilities to the analyte sensors beyond just a first analyte and a second analyte.

FIG. 19 shows a cross-sectional diagram of an illustrative analyte sensor configuration having two working electrodes, a reference electrode and a counter electrode, which is compatible for use in the disclosure herein. As shown, analyte sensor 300 includes working electrodes 304 and 306 disposed upon opposite faces of substrate 302. First active area 310a is disposed upon the surface of working electrode 304, and second active area 310b is disposed upon the surface of working electrode 306. Counter electrode 320 is electrically isolated from working electrode 304 by dielectric layer 322, and reference electrode 321 is electrically isolated from working electrode 306 by dielectric layer 323. Outer dielectric layers 330 and 332 are positioned upon reference electrode 321 and counter electrode 320, respectively. Membrane 340 can overcoat at least active areas 310a and 310b, according to various embodiments, with other components of analyte sensor 300 or the entirety of analyte sensor 300. In certain embodiments, membrane 340 comprises a copolymer of the present disclosure. For example, but not by way of limitation, membrane 340 comprises a copolymer comprising a first monomer, e.g., a styrene, and a second monomer comprising a heterocycle-containing component, e.g., a vinylpyridine, e.g., 4-vinylpyridine.

In certain embodiments, membrane 340 can be continuous but vary compositionally upon active area 310a and/or upon active area 310b in order to afford different permeability values for differentially regulating the analyte flux at each location. For example, but not by way of limitation, the one or more electrodes can be coated with a first polymer membrane portion 340a and/or a second polymer membrane portion 340b. In certain embodiments, different membrane formulations can be sprayed and/or printed onto the opposing faces of analyte sensor 300. Dip coating techniques can also be appropriate, particularly for depositing at least a portion of a bilayer membrane upon one of active areas 310a and 310b. In certain embodiments, membrane 340 can be the same or vary compositionally at active areas 310a and 310b. For example, but not by way of limitation, membrane 340 can include a bilayer overcoating active area 310a and be a homogeneous membrane overcoating active area 310b, or membrane 340 can include a bilayer overcoating active areas 310b and be a homogeneous membrane overcoating active area 310a. In certain embodiments, one of the first polymer membrane portion and the second polymer membrane portion can comprise a bilayer membrane and the other of the first polymer membrane portion and the second polymer membrane portion can comprise a single membrane polymer, according to particular embodiments of the present disclosure. In certain embodiments, an analyte sensor can include more than one membrane 340, e.g., two or more membranes. For example, but not by way of limitation, an analyte sensor can include a membrane that coats the one or more active areas, e.g., 310a and 310b, and an additional membrane that coats the entire sensor as shown in FIG. 19. In such configurations, a bilayer membrane can be formed over the one or more active areas, e.g., 310a and 310b. In certain embodiments, the two membranes can have different polymeric compositions. For example, but not by way of limitation, a first polymer membrane can include a copolymer of the present disclosure and the second polymer membrane can include a different polymer. In certain embodiments, any one of active areas 310a and 310b can comprise an electron transfer agent described herein. In certain embodiments, only one of active areas 310a and 310b can comprise a redox mediator described herein. For example, but not by way of limitation, only active area 310a includes a redox mediator described herein. In certain embodiments, only active area 310b includes a redox mediator described herein. In certain embodiments, both active areas 310a and 310b comprise a redox mediator described herein. In certain embodiments, the redox mediator present in active area 310a is different from the electron transfer agent present in 310b. Alternatively, the redox mediator present in active area 310a is the same electron transfer agent present in 310b.

Alternative sensor configurations having multiple working electrodes and differing from the configuration shown in FIG. 19 can feature a counter/reference electrode instead of separate counter and reference electrodes 320, 321, and/or feature layer and/or membrane arrangements varying from those expressly depicted. For example, and not by the way of limitation, the positioning of counter electrode 320 and reference electrode 321 can be reversed from that depicted in FIG. 19. In addition, working electrodes 304 and 306 need not necessarily reside upon opposing faces of substrate 302 in the manner shown in FIG. 19.

Although suitable sensor configurations can feature electrodes that are substantially planar in character, it is to be appreciated that sensor configurations featuring non-planar electrodes can be advantageous and particularly suitable for use in the disclosure herein. In particular, substantially cylindrical electrodes that are disposed concentrically with respect to one another can facilitate deposition of a mass transport limiting membrane, as described hereinbelow. For example, but not by way of limitation, concentric working electrodes that are spaced apart along the length of a sensor tail can facilitate membrane deposition through sequential dip coating operations, in a similar manner to that described above for substantially planar sensor configurations. FIGS. 20A-20C show perspective views of analyte sensors featuring two working electrodes that are disposed concentrically with respect to one another. It is to be appreciated that sensor configurations having a concentric electrode disposition but lacking a second working electrode are also possible in the present disclosure.

FIG. 20A shows a perspective view of an illustrative sensor configuration in which multiple electrodes are substantially cylindrical and are disposed concentrically with respect to one another about a central substrate. As shown, analyte sensor 400 includes central substrate 402 about which all electrodes and dielectric layers are disposed concentrically with respect to one another. In particular, working electrode 410 is disposed upon the surface of central substrate 402, and dielectric layer 412 is disposed upon a portion of working electrode 410 distal to sensor tip 404. Working electrode 420 is disposed upon dielectric layer 412, and dielectric layer 422 is disposed upon a portion of working electrode 420 distal to sensor tip 404. Counter electrode 430 is disposed upon dielectric layer 422, and dielectric layer 432 is disposed upon a portion of counter electrode 430 distal to sensor tip 404. Reference electrode 440 is disposed upon dielectric layer 432, and dielectric layer 442 is disposed upon a portion of reference electrode 440 distal to sensor tip 404. As such, exposed surfaces of working electrode 410, working electrode 420, counter electrode 430, and reference electrode 440 are spaced apart from one another along longitudinal axis B of analyte sensor 400.

Referring still to FIG. 20A, first active areas 414a and second active areas 414b, which are responsive to different analytes, are disposed upon the exposed surfaces of working electrodes 410 and 420, respectively, thereby allowing contact with a fluid to take place for sensing. Although active areas 414a and 414b have been depicted as three discrete spots in FIG. 20A, it is to be appreciated that fewer or greater than three spots, including a continuous layer of active area, can be present in alternative sensor configurations. In certain embodiments, any one of active areas 414a and 414b can comprise an electron transfer agent described herein. In certain embodiments, only one of active areas 414a and 414b can comprise a redox mediator described herein. For example, but not by way of limitation, only active area 414a includes a redox mediator described herein. In certain embodiments, only active area 414b includes a redox mediator described herein. In certain embodiments, both active areas 414a and 414b comprise a redox mediator described herein. In certain embodiments, the redox mediator present in active area 414a is different from the electron transfer agent present in 414b. Alternatively, the redox mediator present in active area 414a is the same electron transfer agent present in 414b.

In FIG. 20A, sensor 400 is partially coated with membrane 450 upon working electrodes 410 and 420 and active areas 414a and 414b disposed thereon. FIG. 20B shows an alternative sensor configuration in which the substantial entirety of sensor 401 is coated with membrane 450. Membrane 450 can be the same or vary compositionally at active areas 414a and 414b. For example, membrane 450 can include a bilayer overcoating active area 414a and be a homogeneous membrane overcoating active area 414b. In certain embodiments, membrane 450 comprises a copolymer of the present disclosure. For example, but not by way of limitation, membrane 450 comprises a copolymer comprising a first monomer, e.g., a styrene, and a second monomer comprising a heterocycle-containing component, e.g., a vinylpyridine, e.g., 4-vinylpyridine.

It is to be further appreciated that the positioning of the various electrodes in FIGS. 20A and 20B can differ from that expressly depicted. For example, the positions of counter electrode 430 and reference electrode 440 can be reversed from the depicted configurations in FIGS. 20A and 20B. Similarly, the positions of working electrodes 410 and 420 are not limited to those that are expressly depicted in FIGS. 20A and 20B. FIG. 20C shows an alternative sensor configuration to that shown in FIG. 20B, in which sensor 405 contains counter electrode 430 and reference electrode 440 that are located more proximal to sensor tip 404 and working electrodes 410 and 420 that are located more distal to sensor tip 404. Sensor configurations in which working electrodes 410 and 420 are located more distal to sensor tip 404 can be advantageous by providing a larger surface area for deposition of active areas 414a and 414b (five discrete sensing spots illustratively shown in FIG. 20C), thereby facilitating an increased signal strength in some cases. Similarly, central substrate 402 can be omitted in any concentric sensor configuration disclosed herein, wherein the innermost electrode can instead support subsequently deposited layers.

In certain embodiments, an analyte sensor of the present disclosure includes one or more sensing spots (e.g., two or more, three or more, four or more, five or more or six or more) on an in vivo portion, as described herein. For example, but not by way of limitation, FIG. 1B illustrates a cross-sectional diagram of an exemplary analyte sensor according to one or more embodiments of the present disclosure. As shown in FIG. 1B, the analyte sensor can include a sensor tail 100 including: (i) a substrate 102; (ii) a first working electrode 104 on the substrate 102; (ii) a sensing spot 108 disposed upon a surface of the first working electrode for detecting an analyte; (iii) a membrane 110 overcoating at least the sensing spot 108; and (iv) a counter/reference electrode 106 on the substrate 102. In certain embodiments, the substrate 102 can be disposed between the first working electrode 104 and the counter/reference electrode 106. In certain embodiments, the first working electrode 104 and the counter/reference electrode 106 can be located upon the same side of the substrate 102 with a dielectric material interposed therebetween. In certain embodiments, the sensing spot 108 can be disposed as at least one layer upon at least a portion of the first working electrode 104. The sensing spot 108 can include an active area configured to detect an analyte through sensing chemistry (also referred to herein as active chemistry). In certain embodiments, the membrane 110 coats the sensing spot 108. In certain embodiments, the membrane 100 can overcoat some or all of the first working electrode 104 and/or counter/reference electrode 106, or the entirety of the sensor tail 100 of the analyte sensor. One or both faces of the sensor tail 100 of the analyte sensor can be coated with the membrane 110. In certain embodiments, the membrane 110 can include one or more polymeric membrane materials having capabilities of limiting analyte flux to the sensing spot 108 (i.e., the membrane 110 is a mass-limiting membrane having some permeability for the analyte of interest). In certain embodiments, the membrane 110 can be crosslinked with a branched crosslinker in certain particular sensor configurations. The composition and thickness of the membrane 110 can vary to promote a desired analyte flux to the sensing spot 108, thereby providing a desired signal intensity and stability. The analyte sensor can be operable for assaying an analyte by any of coulometric, amperometric, voltammetric, or potentiometric electrochemical detection techniques.

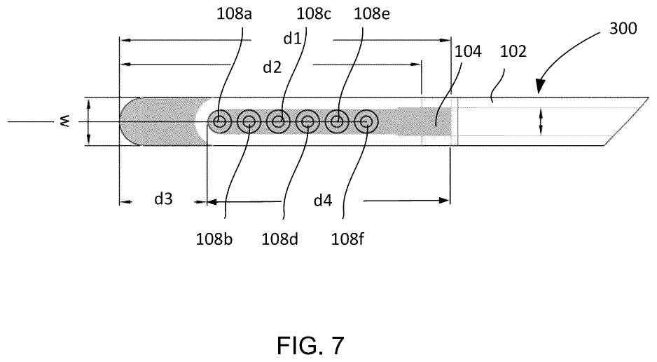

FIG. 2 illustrates a cross-sectional diagram of an analyte sensor 200 according to one or more embodiments of the present disclosure. FIG. 2 is similar to FIG. 1B, except for the presence of a plurality of sensing spots 108a-108f (first sensing spot 108a to sixth sensing spot 108f) on the first working electrode 104. FIG. 7 further illustrates a schematic view of an example sensor tail according to one or more embodiments of the present disclosure. As shown in FIG. 7, an analyte sensor can include a sensor tail 300 including: (i) a substrate 102; (ii) a first working electrode 104 on the substrate 102; (ii) a plurality of sensing spots 108a-108f disposed upon a surface of the first working electrode 104 for detecting an analyte; and (iii) a counter/reference electrode (other side of the substrate, not shown in FIG. 7). The plurality of sensing spots 108a-108f can be responsive to different analytes and are laterally spaced apart from one another on the surface of the first working electrode 104. In certain embodiments, at least some of the plurality of sensing spots 108a-108f can be responsive to a same analyte, and the rest can be responsive to different analytes. In certain embodiments, the membrane (e.g., 110 in FIG. 2) can at least cover a portion of the plurality of sensing spots 108a-108f or all of the plurality of sensing spots 108a-108f. The composition of the membrane (e.g., 110 in FIG. 2) can vary or be compositionally the same at the plurality of sensing spots 108a-108f. The plurality of sensing spots 108a-108f can be configured to detect their corresponding analytes at working electrode potentials that differ from one another.

In certain embodiments, one or more electrodes of an analyte sensor described herein is a wire electrode, e.g., a permeable wire electrode. In certain embodiments, the sensor tail comprises a working electrode and a reference electrode helically wound around the working electrode. In certain embodiments, an insulator is disposed between the working and reference electrodes. In certain embodiments, portions of the electrodes are exposed to allow reaction of the one or more enzymes with an analyte on the electrode. In certain embodiments, each electrode is formed from a fine wire with a diameter of from about 0.001 inches or less to about 0.010 inches or more. In certain embodiments, the working electrode has a diameter of from about 0.001 inches or less to about 0.010 inches or more, e.g., from about 0.002 inches to about 0.008 inches or from about 0.004 inches to about 0.005 inches. In certain embodiments, an electrode is formed from a plated insulator, a plated wire or bulk electrically conductive material. In certain embodiments, the working electrode comprises a wire formed from a conductive material, such as platinum, platinum-iridium, palladium, graphite, gold, carbon, conductive polymer, alloys or the like. In certain embodiments, the conductive material is a permeable conductive material. In certain embodiments, the electrodes can be formed by a variety of manufacturing techniques (e.g., bulk metal processing, deposition of metal onto a substrate or the like), the electrodes can be formed from plated wire (e.g., platinum on steel wire) or bulk metal (e.g., platinum wire). In certain embodiments, the electrode is formed from tantalum wire, e.g., covered with platinum.

In certain embodiments, the reference electrode, which can function as a reference electrode alone, or as a dual reference and counter electrode, is formed from silver, silver/silver chloride or the like. In certain embodiments, the reference electrode is juxtaposed and/or twisted with or around the working electrode. In certain embodiments, the reference electrode is helically wound around the working electrode. In certain embodiments, the assembly of wires can be coated or adhered together with an insulating material so as to provide an insulating attachment.

In certain embodiments, additional electrodes can be included on the in vivo portion of the analyte sensor. For example, but not by way of limitation, an analyte sensor of the present disclosure can include a three-electrode system (a working electrode, a reference electrode and a counter electrode) and/or an additional working electrode (e.g., an electrode for detecting a second analyte). In certain embodiments where the sensor comprises two working electrodes, the two working electrodes can be juxtaposed around which the reference electrode is disposed upon (e.g., helically wound around the two or more working electrodes). In certain embodiments, the two or more working electrodes can extend parallel to each other. In certain embodiments, the reference electrode is coiled around the working electrode and extends towards the distal end (i.e., in vivo end) of the sensor tail. In certain embodiments, the reference electrode extends (e.g., helically) to the exposed region of the working electrode.

In certain embodiments, one or more working electrodes are helically wound around a reference electrode. In certain embodiments where two or more working electrodes are provided, the working electrodes can be formed in a double-, triple-, quad- or greater helix configuration along the length of the sensor tail (for example, surrounding a reference electrode, insulated rod or other support structure). In certain embodiments, the electrodes, e.g., two or more working electrodes, are coaxially formed. For example, but not by way limitation, the electrodes all share the same central axis.

In certain embodiments, the working electrode comprises a tube with a reference electrode disposed or coiled inside, including an insulator therebetween. Alternatively, the reference electrode comprises a tube with a working electrode disposed or coiled inside, including an insulator therebetween. In certain embodiments, a polymer (e.g., insulating) rod is provided, wherein the one or more electrodes (e.g., one or more electrode layers) are disposed upon (e.g., by electro-plating). In certain embodiments, a metallic (e.g., steel or tantalum) rod or wire is provided, coated with an insulating material (described herein), onto which the one or more working and reference electrodes are disposed upon. For example, but not by way of limitation, the present disclosure provides a sensor, e.g., a sensor tail, that comprises one or more tantalum wires, where a conductive material is disposed upon a portion of the one or more tantalum wires to function as a working electrode. In certain embodiments, the platinum-clad tantalum wire is covered with an insulating material, where the insulating material is partially covered with a silver/silver chloride composition to function as a reference and/or counter electrode.

In certain embodiments where an insulator is disposed upon the working electrode (e.g., upon the platinum surface of the electrode), a portion of the insulator can be stripped or otherwise removed to expose the electroactive surface of the working electrode. For example, but not by way of limitation, a portion of the insulator can be removed by hand, excimer lasing, chemical etching, laser ablation, grit-blasting or the like. Alternatively, a portion of the electrode can be masked prior to depositing the insulator to maintain an exposed electroactive surface area. In certain embodiments, the portion of the insulator that is stripped and/or removed can be from about 0.1 mm or less to about 2 mm or more in length, e.g., from about 0.5 mm to about 0.75 mm in length. In certain embodiments, the insulator is a non-conductive polymer. In certain embodiments, the insulator comprises parylene, fluorinated polymers, polyethylene terephthalate, polyvinylpyrrolidone, polyurethane, polyimide and other non-conducting polymers. In certain embodiments, glass or ceramic materials can also be used in the insulator layer. In certain embodiments, the insulator comprises parylene. In certain embodiments, the insulator comprises a polyurethane. In certain embodiments, the insulator comprises a polyurethane and polyvinylpyrrolidone.

ii. Sensing Chemistry

The analyte sensors of the present disclosure can include one or more enzymes for detecting one or more analytes. In certain embodiments, an active area of a presently disclosed analyte sensor, e.g., disposed upon a working electrode, can be configured for detecting one or more analytes. In certain embodiments, an active area comprises one or more enzymes for detecting an analyte. In certain embodiments, an analyte sensor of the present disclosure can include more than one active area, where each active area is configured to detect the same analyte or different analytes. In certain embodiments, the sensor does not include an enzyme and the analyte is directly oxidized at the working electrode.

In certain embodiments, an active area of a sensor of the present disclosure can comprise one or more enzymes to detect an analyte including, but not limited to, glucose, lactate, ketones (e.g., ketone bodies), glutamine, alcohols, aspartate, asparagine, glutamate, creatinine, hematocrit, acetoacetate, fructosamine, amylase, bilirubin, cholesterol, chorionic gonadotropin, creatine kinase (e.g., CK-MB), creatine, DNA, RNA, growth factors, growth hormones, hormones (e.g., thyroid stimulating hormone), steroids, vitamins (e.g., ascorbic acid), uric acid, neurochemicals (e.g., acetylcholine, norepinephrine and dopamine), oxygen, albumin, hemoglobin A1C, alkaline phosphatase, alanine transaminase, aspartate aminotransferase, blood urea nitrogen, sarcosine, prostate-specific antigen, prothrombin, thrombin, troponin, pyruvate, acetaldehyde, ascorbate, galactose, L-xylono-1,4-lactone, glutathione disulfide, hydrogen peroxide, linoleate, 1,3-bisphosphoglycerate, 6-phospho-D-glucono-1,5-lactone, hemoglobin, pharmaceutical drugs (e.g., antibiotics (e.g., gentamicin, vancomycin and the like), digitoxin, digoxin, theophylline, insulin and warfarin), drugs of abuse (e.g., analgesics, depressants, stimulants and hallucinogens), metal ions (e.g., potassium, sodium, calcium, magnesium, manganese, iron, cobalt, molybdenum, zinc and chlorine), pH, carbonate, phosphate, sulfate, fatty acids and antibodies.

In certain embodiments, the analyte is glucose, lactate, ketones, glutamate, creatinine, sarcosine and/or ascorbate.

In certain embodiments, the analyte is glucose.

In certain embodiments, the analyte is ketones.

In certain embodiments, the analyte is lactate.

In certain embodiments, the analyte is glutamate.

In certain embodiments, the one or more enzymes in an active area of a sensor of the present disclosure can be used in detecting glutamate, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, and uric acid.

In certain embodiments, enzymes for utilization in detecting glucose, lactate, ketones, creatinine, alcohol, e.g., ethanol, and/or the like can be included in one or more active areas and/or one or more sensing spots of an analyte sensor disclosed herein, e.g., as shown in FIGS. 1A and 1B. In certain embodiments, enzymes for utilization in detecting glucose, lactate, ketones, creatinine, alcohol, e.g., ethanol, and/or the like can be included in two or more sensing spots of an analyte sensor disclosed herein, e.g., as shown in FIG. 2. In certain embodiments, the one or more enzymes can include multiple enzymes, e.g., an enzyme system, that are collectively responsive to the analyte.

In certain embodiments, the enzyme can be an oxidoreductase. In certain embodiments, the oxidoreductase can be an enzyme belonging to enzyme class 1. For example, but not by way of limitation, the enzyme can belong to enzyme class 1.1 (e.g. 1.1.1 or 1.1.3), enzyme class 1.4 (e.g., 1.4.3) or enzyme class 1.5. In certain embodiments, the enzyme can be a NAD(P)+-dependent dehydrogenase. In certain embodiments, the enzyme can be a flavin adenine dinucleotide (FAD)-dependent oxidoreductase. In certain embodiments, the enzyme can be a hydrolase. In certain embodiments, the hydrolase can be an enzyme belonging to enzyme class 3. For example, but not by way of limitation, the enzyme can belong to enzyme class 3.5, e.g., 3.5.2 or 3.5.3.

In certain embodiments, an active area of a sensor of the present disclosure (e.g., one or more sensing spots of an active area of an analyte sensor of the present disclosure) can include one or more enzymes that can be utilized to detect glucose. For example, but not by way of limitation, an analyte sensor of the present disclosure can include one or more sensing spots, e.g., sensing spot 108a, sensing spot 108b, sensing spot 108c, etc., that includes one or more enzymes for detecting glucose. In certain embodiments, the analyte sensor can include one or more sensing spots including a glucose oxidase and/or a glucose dehydrogenase for detecting glucose. In certain embodiments, the analyte sensor can include one or more sensing spots including a glucose oxidase. In certain embodiments, the glucose dehydrogenase can be a pyrroloquinoline quinone (PQQ) or a cofactor-dependent glucose dehydrogenase, e.g., flavin adenine dinucleotide (FAD)-dependent glucose dehydrogenase or nicotinamide adenine dinucleotide (NAD)-dependent glucose dehydrogenase. In certain embodiments, the active area (e.g., the one or more sensing spots) can further include diaphorase. In certain embodiments, the active area (e.g., the one or more sensing spots) can further include diaphorase. In certain embodiments, the enzyme for detecting glucose is an FAD-dependent glucose oxidase.

In certain embodiments, an active area of a sensor of the present disclosure (e.g., one or more sensing spots of an active area of an analyte sensor of the present disclosure) can include one or more enzymes that can be utilized to detect ketones. For example, but not by way of limitation, an analyte sensor of the present disclosure can include one or more sensing spots, e.g., sensing spot 108d, that includes one or more enzymes, e.g., an enzyme system, for detecting ketones. In certain embodiments, a ketones-responsive active area can include an enzyme system comprising multiple enzymes that are capable of acting in concert to facilitate detection of ketones, as described in U.S. Patent Publication No. 2020/0237275 (the contents of which are incorporated by reference herein in their entirety). In certain embodiments, the analyte sensor can include one or more sensing spots including β-hydroxybutyrate dehydrogenase. In certain embodiments, the analyte sensor can include one or more sensing spots including β-hydroxybutyrate dehydrogenase and diaphorase for detecting ketones. In certain embodiments, the active area (e.g., the one or more sensing spots) can further include diaphorase.

In certain embodiments, an active area of a sensor of the present disclosure (e.g., one or more sensing spots of an active area of an analyte sensor of the present disclosure) can include one or more enzymes that can be utilized to detect lactate. For example, but not by way of limitation, an analyte sensor of the present disclosure can include one or more sensing spots, e.g., sensing spot 108e, that includes one or more enzymes, e.g., an enzyme system, for detecting lactate. In certain embodiments, a lactate-responsive active area (e.g., one or more sensing spots) can include an enzyme system comprising multiple enzymes that are capable of acting in concert to facilitate detection of lactate, as described in U.S. Publication No. 2019/0320947 (the contents of which are incorporated by reference herein in their entirety). In certain embodiments, the analyte sensor can include one or more sensing spots including a lactate dehydrogenase. In certain embodiments, the analyte sensor can include one or more sensing spots including a lactate oxidase. In certain embodiments, the active area (e.g., the one or more sensing spots) can further include diaphorase.

In certain embodiments, an active area of a sensor of the present disclosure (e.g., one or more sensing spots of an active area of an analyte sensor of the present disclosure) can include one or more enzymes that can be utilized to detect alcohol. For example, but not by way of limitation, an analyte sensor of the present disclosure can include one or more sensing spots, e.g., sensing spot 108e, that includes one or more enzymes, e.g., an enzyme system, for detecting alcohol. In certain embodiments, an ethanol-responsive active area can include an enzyme system comprising multiple enzymes that are capable of acting in concert to facilitate detection of ethanol, as in U.S. Patent Publication No. 2020/0237277 (the contents of which are incorporated by reference herein in their entirety). In certain embodiments, the analyte sensor can include one or more sensing spots including an alcohol dehydrogenase or a ketoreductase.

In certain embodiments, an active area of a sensor of the present disclosure (e.g., one or more sensing spots of an active area of an analyte sensor of the present disclosure) can include one or more enzymes that can be utilized to detect creatinine. For example, but not by way of limitation, an analyte sensor of the present disclosure can include one or more sensing spots, e.g., sensing spot 108e, that includes one or more enzymes, e.g., an enzyme system, for detecting creatinine. In certain embodiments, a creatinine-responsive active area (e.g., one or more sensing spots) can include an enzyme system comprising multiple enzymes that are capable of acting in concert to facilitate detection of creatinine, e.g., as described in U.S. Patent Publication No. 2020/0241015 (the contents of which are incorporated by reference herein in their entirety). In certain embodiments, the analyte sensor can include one or more sensing spots including an amidohydrolase, creatine amidinohydrolase and/or sarcosine oxidase.

In certain embodiments, an active area of a sensor of the present disclosure (e.g., one or more sensing spots of an active area of an analyte sensor of the present disclosure) can include one or more enzymes that can be utilized to detect glutamate. For example, but not by way of limitation, an analyte sensor of the present disclosure can include one or more sensing spots, e.g., sensing spot 108e, that includes one or more enzymes, e.g., an enzyme system, for detecting glutamate. In certain embodiments, the analyte sensor can include one or more sensing spots including a glutamate dehydrogenase or a glutamate oxidase.

In certain embodiments, an active area of the present disclosure can include one or more sensing spots, where each sensing spot can include the one or more enzymes for detecting an analyte. For example, but not by way of limitation, an analyte sensor disclosed herein can include two or more sensing spots, e.g., sensing spots 108a-108f, with each sensing spot including at least one enzyme for detecting an analyte. In certain embodiments, each an active area (e.g., each sensing spot) can be configured to detect the same analyte or a different analyte. For example, but not by way of limitation, an analyte sensor of the present disclosure can include a first sensing spot that includes a first enzyme (or first enzyme system) for detecting a first analyte and a second sensing spot that includes a second enzyme (or second enzyme system) for detecting a second analyte, and so on. In certain embodiments, the first sensing spot and the second sensing spot can be utilized to detect the same analyte, where the first sensing spot and the second sensing spot can include different enzymes (or enzyme system) or the same enzyme (or enzyme system) for detecting the analyte.

In certain embodiments, an active area (e.g., an analyte-responsive sensing spot) can further include a stabilizing agent, e.g., for stabilizing the one or more enzymes. For example, but not by way of limitation, the stabilizing agent can be an albumin, e.g., a serum albumin. Non-limiting examples of serum albumins can include bovine serum albumin and human serum albumin. In certain embodiments, the stabilizing agent can be a human serum albumin. In certain embodiments, the stabilizing agent can a bovine serum albumin.

In certain embodiments, an active area (e.g., an analyte-responsive sensing spot) can further include a cofactor or coenzyme for one or more enzymes present in the analyte-responsive sensing spot. In certain embodiments, the cofactor can be nicotinamide adenine dinucleotide (NAD) or nicotinamide adenine dinucleotide phosphate (NADP). In certain embodiments, the coenzyme can be NAD.

In certain embodiments, a sensor of the present disclosure does not include an analyte-responsive active area comprising an enzyme. In certain embodiments, a sensor of the present disclosure includes a working electrode that does not have an enzyme disposed upon the working electrode or includes an inactive enzyme, e.g., an enzyme that lacks enzymatic activity (e.g., for the analyte of interest), disposed upon the working electrode. In certain embodiments, such a sensor can be used to detect an analyte that can be directly oxidized at the working electrode. For example, but not by way of limitation, a sensor of the present disclosure for detecting ascorbate does not include an enzyme on the working electrode. In certain embodiments, ascorbate is directly oxidized at the working electrode resulting in a signal that correlates to the level of ascorbate in the biological fluid contacting the sensor.

In certain embodiments, a working electrode that does not include an enzyme or includes an inactive enzyme can be used for detecting a background signal. In certain embodiments, the background signal includes a signal that is caused by chemical species other than the analyte of interest present in the sample, e.g., signal caused by an interferent. In certain embodiments, the background signal is a signal caused by one or more interferents. Non-limiting examples of interferents include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea and uric acid. In certain embodiments, the background signal can be used to calibrate, filter and/or normalize the signal obtained from a second working electrode (which is configured for detecting an analyte) present on the same analyte sensor. In certain embodiments, the signal from the working electrode that does not have enzyme (or has inactive enzyme) can be subtracted from the signal obtained from a working electrode that is configured to detect an analyte to determine the signal contribution from the analyte.

In certain embodiments, an analyte sensor disclosed herein can include an electron transfer agent. For example, but not by way of limitation, an active area (e.g., one or more sensing spots of an analyte sensor) can include an electron transfer agent. In certain embodiments, an analyte sensor can include one sensing spot that includes an electron transfer agent and a second sensing spot that does not include an electron transfer agent. In certain embodiments, an analyte sensor can include a plurality of sensing spots, where the plurality of sensing spots can include an electron transfer agent. In certain embodiments, the presence of an electron transfer agent in a sensing spot can depend on the enzyme or enzyme system used to detect the analyte and/or the composition of the working electrode.

Suitable electron transfer agents for use in the presently disclosed analyte sensors can facilitate conveyance of electrons to the adjacent working electrode after an analyte undergoes an enzymatic oxidation-reduction reaction within the corresponding sensing spot, thereby generating a current that is indicative of the presence of that particular analyte. The amount of current generated is proportional to the quantity of analyte that is present.

In certain embodiments, suitable electron transfer agents can include electroreducible and electrooxidizable ions, complexes, or molecules (e.g., quinones) having oxidation-reduction potentials that are a few hundred millivolts above or below the oxidation-reduction potential of the standard calomel electrode. In certain embodiments, the redox mediators can include osmium complexes and other transition metal complexes, such as those described in U.S. Pat. Nos. 6,134,461 and 6,605,200, which are incorporated herein by reference in their entireties. Additional examples of suitable redox mediators can include those described in U.S. Pat. Nos. 6,736,957, 7,501,053 and 7,754,093, the disclosures of each of which are also incorporated herein by reference in their entireties. Other examples of suitable redox mediators can include metal compounds or complexes of ruthenium, osmium, iron (e.g., polyvinylferrocene or hexacyanoferrate), or cobalt, including metallocene compounds thereof, for example. Suitable ligands for the metal complexes can include, for example, bidentate or higher denticity ligands such as, for example, bipyridine, biimidazole, phenanthroline, or pyridyl(imidazole). Other suitable bidentate ligands can include, for example, amino acids, oxalic acid, acetylacetone, diaminoalkanes, or o-diaminoarenes. Any combination of monodentate, bidentate, tridentate, tetradentate or higher denticity ligands can be present in a metal complex, e.g., osmium complex, to achieve a full coordination sphere. In certain embodiments, the electron transfer agent is an osmium complex. In certain embodiments, the electron transfer agent is osmium complexed with bidentate ligands. In certain embodiments, the electron transfer agent is osmium complexed with tridentate ligands.

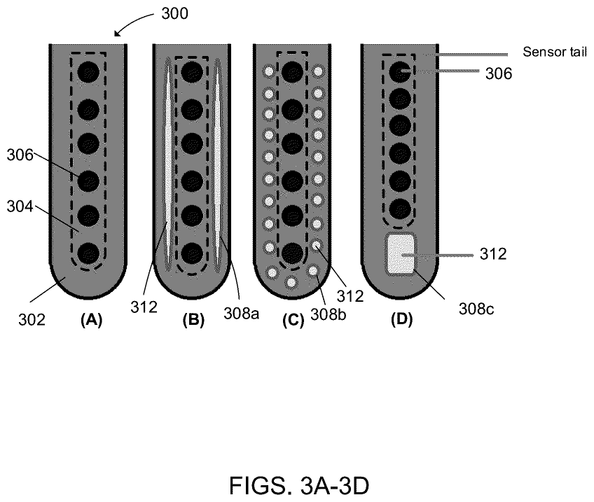

In certain embodiments, electron transfer agents disclosed herein can comprise suitable functionality to promote covalent bonding to a polymer (also referred to herein as a polymeric backbone) within the active areas as discussed further below. For example, but not by way of limitation, an electron transfer agent for use in the present disclosure can include a polymer-bound electron transfer agent, e.g., a redox polymer. Suitable non-limiting examples of polymer-bound electron transfer agents include those described in U.S. Pat. Nos. 8,444,834, 8,268,143 and 6,605,201 and U.S. Patent Publication No. 2022/0202326, the disclosures of which are incorporated herein by reference in their entirety. In certain embodiments, the electron transfer agent is a bidentate osmium complex bound to a polymer described herein, e.g., a polymeric backbone described in Section 4 below. In certain embodiments, the electron transfer agent is a tridentate osmium complex bound to a polymer described herein, e.g., a polymeric backbone described in Section 4 below. In certain embodiments, the polymer-bound electron transfer agent shown in FIG. 3 of U.S. Pat. No. 8,444,834 (referred to as “X7”) can be used in a sensor of the present disclosure.

In certain embodiments, one or more working electrodes of an analyte sensor of the present disclosure does not have a redox mediator disposed upon the working electrode. In certain embodiments, one or more working electrodes of an analyte sensor of the present disclosure does not have a redox mediator or an enzyme disposed upon the working electrode. In certain embodiments, such working electrodes can be used to detect an analyte (or a reaction product) that can be directly oxidized at the working electrode. For example, but not by way of limitation, a working electrode that has an active that does not include a redox mediator can be used to detect an analyte, e.g., glucose, by oxidation of hydrogen peroxide on the working electrode.

iii. Membrane

In certain embodiments, an analyte sensor of the present disclosure further includes a membrane covering at least a portion of the sensing layer. For example, but not by way of limitation, the membrane can function as a mass-limiting membrane and/or to improve biocompatibility. In certain embodiments, membrane 110 can overcoat at least a portion of the active area as shown in FIG. 1A. In certain embodiments, the membrane can overcoat at least a portion of a plurality of sensing spots, e.g., plurality of sensing spots 108a-108f as shown in FIG. 2.

A mass-limiting membrane can act as a diffusion-limiting barrier to reduce the rate of mass transport of the analyte, e.g., glucose, an alcohol, a ketone, or lactate, when the sensor is in use. For example, but not by way of limitation, limiting access of an analyte, e.g., glucose, to the sensing spot with a mass-limiting membrane can aid in avoiding sensor overload (saturation), thereby improving detection performance and accuracy. In certain embodiments, the mass-limiting layer can limit the flux of an analyte to the working electrode in an electrochemical sensor so that the sensor is linearly responsive over a large range of analyte concentrations.

In certain embodiments, the mass-limiting membrane can be homogeneous and can be single-component (contain a single membrane polymer). In certain embodiments, the mass-limiting membrane can be multi-component (contain two or more different membrane polymers). In certain embodiments, the multi-component membrane can be present as a bilayer membrane or as a homogeneous admixture of two or more membrane polymers. A homogeneous admixture can be deposited by combining the two or more membrane polymers in a solution and then depositing the solution upon a working electrode, e.g., by dip coating.

In certain embodiments, the mass-limiting membrane can include two or more layers, e.g., a bilayer or tri-layer membrane. In certain embodiments, each layer can include a different polymer or the same polymer at different concentrations or thicknesses.

In certain embodiments, a mass-limiting membrane can include polymers containing heterocyclic nitrogen groups. In certain embodiments, a mass-limiting membrane can include a polyvinylpyridine-based polymer. Non-limiting examples of polyvinylpyridine-based polymers are disclosed in U.S. Patent Publication No. 2003/0042137, the content of which is incorporated by reference herein in its entirety. In certain embodiments, the polyvinylpyridine-based polymer has a molecular weight from about 50 kD to about 500 kD, e.g., from about 50 kD to about 200 kD.

In certain embodiments, a mass-limiting membrane can include a polyvinylpyridine (e.g., poly(2-vinylpyridine) or poly(4-vinylpyridine)), a polyvinylimidazole, a polyvinylpyridine copolymer (e.g., a copolymer of vinylpyridine and styrene), a polyacrylate, a polyurethane, a polyether urethane, a silicone, a polytetrafluoroethylene, a polyethylene-co-tetrafluoroethylene, a polyolefin, a polyester, a polycarbonate, a biostable polytetrafluoroethylene, homopolymers, copolymers or terpolymers of polyurethanes, a polypropylene, a polyvinylchloride, a polyvinylidene difluoride, a polybutylene terephthalate, a polymethylmethacrylate, a polyether ether ketone, cellulosic polymers, polysulfones and block copolymers thereof including, for example, di-block, tri-block, alternating, random and graft copolymers or a chemically related material and the like.

In certain embodiments, a mass-limiting membrane can include a polyvinylpyridine (e.g., poly(4-vinylpyridine) and/or poly(2-vinylpyridine)). In certain embodiments, a mass-limiting membrane can include poly(4-vinylpyridine). In certain embodiments, a mass-limiting membrane can include a copolymer of vinylpyridine and styrene. In certain embodiments, the mass-limiting membrane can include a polyvinylpyridine-co-styrene copolymer. For example, but not by way of limitation, a polyvinylpyridine-co-styrene copolymer can include a polyvinylpyridine-co-styrene copolymer in which a portion of the pyridine nitrogen atoms are functionalized with a non-crosslinked polyethylene glycol tail and a portion of the pyridine nitrogen atoms were functionalized with an alkylsulfonic acid group, e.g., a propylsulfonic acid. In certain embodiments, a derivatized polyvinylpyridine-co-styrene copolymer for use as a membrane polymer can be the 10Q5 polymer as described in U.S. Pat. No. 8,761,857, the content of which is incorporated by reference herein in its entirety.

In certain embodiments, a suitable copolymer of vinylpyridine and styrene can have a styrene content ranging from about 0.01% to about 50% mole percent (mer %), or from about 0.05% to about 45% mole percent, or from about 0.1% to about 40% mole percent, or from about 0.5% to about 35% mole percent, or from about 1% to about 30% mole percent, or from about 2% to about 25% mole percent, or from about 5% to about 20% mole percent. In certain embodiments, a copolymer of vinylpyridine and styrene can include a styrene content ranging from about 2% to about 25% mole percent. Substituted styrene can be utilized similarly and in similar amounts.

In certain embodiments, a suitable copolymer of vinylpyridine and styrene can have a weight average molecular weight of 5 kD or more, or about 10 kD or more, or about 15 kD or more, or about 20 kD or more, or about 25 kD or more, or about 30 kD or more, or about 40 kD or more, or about 50 kD or more, or about 75 kD or more, or about 90 kD or more, about 100 kD or more or about 110 kD or more. In non-limiting examples, a suitable copolymer of vinylpyridine and styrene can have a weight average molecular weight ranging from about 5 kD to about 150 kD, or from about 10 kD to about 125 kD, or from about 15 kD to about 100 kD, or from about 20 kD to about 80 kD, or from about 25 kD to about 75 kD, or from about 30 kD to about 60 kD. In certain embodiments, a copolymer of vinylpyridine and styrene can have a weight average molecular weight ranging from about 10 kD to about 125 kD.

In certain embodiments, a mass-limiting membrane can further include a silicone polymer, e.g., a poly dimethylsiloxane (PDMS). For example, but not by way of limitation, a mass-limiting membrane can include a polyvinylpyridine-co-styrene copolymer (e.g., a derivatized polyvinylpyridine-co-styrene copolymer) and a silicone polymer (e.g., a polydimethylsiloxane (PDMS)).

iv. Interference Domain

In certain embodiments, the analyte sensor of the present disclosure can further include an interference domain. For example, but not by way of limitation, a sensor tail (i.e., an in vivo portion) 100 or 200 of an analyte sensor can further comprise an interference domain. In certain embodiments, the interference domain can include a polymer domain that restricts the flow of one or more interferants, e.g., to the surface of the working electrode. In certain embodiments, the interference domain can function as a molecular sieve that allows analytes and other substances that are to be measured by the working electrode to pass through, while preventing passage of other substances such as interferents. In certain embodiments, the interferents can affect the signal obtained at the working electrode. Non-limiting examples of interferents can include acetaminophen, ascorbate, ascorbic acid, bilirubin, cholesterol, creatinine, dopamine, ephedrine, ibuprofen, L-dopa, methyldopa, salicylate, tetracycline, tolazamide, tolbutamide, triglycerides, urea, and uric acid.

In certain embodiments, the interference domain is located between the working electrode and the active area, e.g., one or more sensing spots. In certain embodiments, non-limiting examples of polymers that can be utilized in the interference domain can include polyurethanes, polymers having pendant ionic groups, and polymers having controlled pore size. In certain embodiments, the interference domain can be formed from one or more cellulosic derivatives.

Non-limiting examples of cellulosic derivatives include polymers such as cellulose acetate, cellulose acetate butyrate, 2-hydroxyethyl cellulose, cellulose acetate phthalate, cellulose acetate propionate, cellulose acetate trimellitate and the like.

In certain embodiments, the interference domain is part of the mass-limiting membrane and not a separate membrane. In certain embodiments, the interference domain is located between the one or more sensing spots and the mass-limiting membrane.

In certain embodiments, the interference domain can include a thin, hydrophobic membrane that is non-swellable and restricts diffusion of high molecular weight species. For example, but not by way of limitation, the interference domain can be permeable to relatively low molecular weight substances, such as hydrogen peroxide, while restricting the passage of higher molecular weight substances, such as ketones, glucose, acetaminophen and/or ascorbic acid.

### IV. Therapeutic Agent-Containing Polymer Compositions

The present disclosure provides therapeutic agent-containing polymer compositions that can be incorporated into an analyte sensor, e.g., an implantable analyte sensor, described herein. For example, but not by way of limitation, an analyte sensor of the present disclosure, e.g., a sensor tail 100 or 200 (i.e., in vivo portion) of an analyte sensor described herein, includes a therapeutic agent-containing polymer composition. The one or more therapeutic agents are released in close proximity to an analyte sensor in vivo. The incorporation of a therapeutic agent within the analyte sensor itself or the delivery of a therapeutic composition in close proximity to the sensor at its in vivo location allows targeted delivery of the therapeutic agent to the tissue around (e.g., surrounding) the implantation site and the analyte sensor.

In certain embodiments, a therapeutic agent-containing polymer composition is also referred to herein as a drug-eluting membrane and a second polymer membrane.

The present disclosure further provides sharps for inserting an analyte sensor into a subject. For example, but not by way of limitation, the present disclosure provides a sharp that is coated with a therapeutic agent-containing polymer composition described herein.

In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to prevent and/or reduce the late sensor attenuation.

In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to reduce and/or minimize sensor signal inaccuracies or in vivo sensor failure, e.g., due to FBR.

Non-limiting examples of therapeutic agents that can be included in a therapeutic agent-containing polymer composition of the present disclosure are disclosed in Section II. For example, but not by way of limitation, the therapeutic agent can be an anti-inflammatory agent. In certain embodiments, the anti-inflammatory agent can be a non-steroidal anti-inflammatory agent. In certain embodiments, the anti-inflammatory agent can be a steroidal anti-inflammatory agent, e.g., a corticosteroid. Non-limiting examples of anti-inflammatory agents can include triamcinolone, betamethasone, dexamethasone, hydrocortisone, prednisone, methylprednisolone, fludrocortisone, acetylsalicylic acid, isobutylphenylpropanoic acid or a derivative thereof, and/or a salt thereof or a prodrug thereof. Non-limiting salt forms can include pharmaceutically acceptable salts including acetate and phosphate salts.

In certain embodiments, the anti-inflammatory agent is dexamethasone.

In certain embodiments, the anti-inflammatory agent can be a salt of dexamethasone.

In certain embodiments, the anti-inflammatory agent can be a derivative of dexamethasone.

In certain embodiments, the dexamethasone derivative and/or dexamethasone salt form can be dexamethasone acetate.

In certain embodiments, the dexamethasone derivative and/or dexamethasone salt form can be dexamethasone sodium phosphate.

In certain embodiments, the therapeutic agent-containing polymer composition can include an amount of the therapeutical agent, e.g., dexamethasone or a derivative thereof, in a range of 0.01 wt %-50 wt % based on the total weight of the therapeutic agent-containing polymer composition. In certain embodiments, the therapeutic agent-containing polymer composition can include an amount of the therapeutical agent, e.g. dexamethasone or a derivative thereof, in a range of 0.01 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 1 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 5 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 10 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 20 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 30 wt %-40 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 5 wt %-20 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 5 wt %-10 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-10 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-20 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-30 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 10 wt %-20 wt % based on the total weight of the therapeutic agent-containing polymer composition.

In certain embodiments, the therapeutic agent-containing polymer composition can include an amount of the therapeutical agent, e.g., dexamethasone or a derivative thereof, in a range of 0.01 wt %-50 wt % based on the total weight of the therapeutic agent-containing polymer composition.

In certain embodiments, the therapeutic agent-containing polymer composition can include an amount of the therapeutic agent, e.g., dexamethasone or a derivative thereof, in a range of about 1 wt %-10 wt % based on the total weight of the therapeutic agent-containing polymer composition.

In certain embodiments, the therapeutic agent-containing polymer composition can include an amount of the therapeutic agent, e.g., dexamethasone or a derivative thereof, that is no greater than about 50 wt % based on the total weight of the polymer (e.g., copolymer) in the composition. In certain embodiments, the therapeutic agent-containing polymer composition can include an amount of the therapeutic agent, e.g. dexamethasone or a derivative thereof, that is no greater than about 45 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 40 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 35 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 30 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 25 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 20 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 15 wt % based on the total weight of the polymer (e.g., copolymer) in the composition. In certain embodiments, the therapeutic agent-containing polymer composition can include an amount of the therapeutic agent, e.g., dexamethasone or a derivative thereof, that is no greater than about 10 wt % based on the total weight of the polymer (e.g., copolymer) in the composition.

In certain embodiments, the therapeutic agent-containing polymer composition can include from about 0.0005 mg to about 0.2 mg of the therapeutic agent, e.g., dexamethasone, or any values in between. In certain embodiments, the therapeutic agent-containing polymer composition can include about 0.0005 mg, about 0.001 mg, about 0.005 mg, about 0.01 mg, about 0.05 mg, about 0.1 mg or about 0.2 mg of the therapeutic agent, e.g., dexamethasone. In certain embodiments, the therapeutic agent-containing polymer composition can include from about 0.0005 mg to about 0.1 mg, about 0.0005 mg to about 0.05 mg, about 0.0005 mg to about 0.01 mg, about 0.0005 mg to about 0.005 mg or about 0.0005 mg to about 0.001 mg of the therapeutic agent, e.g., dexamethasone. In certain embodiments, the therapeutic agent-containing polymer composition can include from about 0.0005 mg to about 0.1 mg. In certain embodiments, the therapeutic agent-containing polymer composition can include from about 0.0005 mg to about 0.01 mg. In certain embodiments, the therapeutic agent-containing polymer composition can include from about 0.001 mg to about 0.1 mg. In certain embodiments, the therapeutic agent-containing polymer composition can include from about 0.001 mg to about 0.01 mg. In certain embodiments, the therapeutic agent-containing polymer composition can include from about 0.001 mg to about 0.005 mg. In certain embodiments, the therapeutic agent-containing polymer composition can include from about 0.001 mg to about 0.003 mg. In certain embodiments, the therapeutic agent-containing polymer composition can include from about 0.1 μg to about 200 μg of the therapeutic agent, e.g., from about 0.5 μg to about 200 μg, from about 1 μg to about 200 μg, from about 1.5 μg to about 200 μg, from about 2.0 μg to about 200 μg, from about 2.5 μg to about 200 μg, from about 3 μg to about 200 μg, from about 4 μg to about 200 μg, from about 5 μg to about 200 μg, from about 10 μg to about 200 μg, from about 15 μg to about 200 μg, from about 20 μg to about 200 μg, from about 25 μg to about 200 μg, from about 30 μg to about 200 μg, from about 35 μg to about 200 μg, from about 40 μg to about 200 μg, from about 45 μg to about 200 μg, from about 50 μg to about 200 μg, from about 55 μg to about 200 μg, from about 60 μg to about 200 μg, from about 65 μg to about 200 μg, from about 70 μg to about 200 μg, from about 75 μg to about 200 μg, from about 80 μg to about 200 μg, from about 85 μg to about 200 μg, from about 90 μg to about 200 μg, from about 95 μg to about 200 μg, from about 100 μg to about 200 μg, from about 110 μg to about 200 μg, from about 120 μg to about 200 μg, from about 130 μg to about 200 μg, from about 140 μg to about 200 μg, from about 150 μg to about 200 μg, from about 160 μg to about 200 μg, from about 170 μg to about 200 μg, from about 180 μg to about 200 μg, from about 190 μg to about 200 μg, from about 0.1 μg to about 190 μg, from about 0.1 μg to about 180 μg, from about 0.1 μg to about 170 μg, from about 0.1 μg to about 160 μg, from about 0.1 μg to about 150 μg, from about 0.1 μg to about 140 μg, from about 0.1 μg to about 130 μg, from about 0.1 μg to about 120 μg, from about 0.1 μg to about 110 μg, from about 0.1 μg to about 100 μg, from about 1 μg to about 150 μg, from about 5 μg to about 150 μg or from about 5 μg to about 120 μg. In certain embodiments, the therapeutic agent-containing polymer composition can include from about 0.1 μg to about 20 μg of the therapeutic agent. In certain embodiments, the therapeutic agent-containing polymer composition can include from about 1 μg to about 100 μg of the therapeutic agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In certain embodiments, the therapeutic agent-containing polymer composition can include from about 1 μg to about 20 μg of the therapeutic agent. In certain embodiments, the therapeutic agent-containing polymer composition can include from about 5 μg to about 20 μg of the therapeutic agent. In certain embodiments, the therapeutic agent-containing polymer composition can include from about 1 μg to about 30 μg of the therapeutic agent. In certain embodiments, the therapeutic agent-containing polymer composition can include from about 5 μg to about 30 μg of the therapeutic agent. In certain embodiments, the therapeutic agent-containing polymer composition can include from about 0.1 μg to about 15 μg of the therapeutic agent. In certain embodiments, the therapeutic agent-containing polymer composition can include from about 0.1 μg to about 10 μg of the therapeutic agent. In certain embodiments, the therapeutic agent-containing polymer composition can include from about 0.1 μg to about 5 μg of the therapeutic agent.

In certain embodiments, the therapeutic agent-containing polymer composition can include from about 0.01 μg to about 5 μg of the therapeutic agent.

In certain embodiments, the therapeutic agent-containing polymer composition can include less than about 50 μg of the therapeutic agent. In certain embodiments, the therapeutic agent-containing polymer composition can include less than about 40 μg of the therapeutic agent. In certain embodiments, the therapeutic agent-containing polymer composition can include less than about 30 μg of the therapeutic agent. In certain embodiments, the therapeutic agent-containing polymer composition can include less than about 20 μg of the therapeutic agent. In certain embodiments, the therapeutic agent-containing polymer composition can include less than about 10 μg of the therapeutic agent. In certain embodiments, the therapeutic agent-containing polymer composition can include less than about 5 μg of the therapeutic agent.

In certain embodiments, the therapeutic agent-containing polymer composition can continuously release the therapeutic agent at a set or predetermined drug delivery rate to reduce and/or minimize sensor signal inaccuracies or in vivo sensor failure, e.g., due to FBR. In certain embodiments, the therapeutic agent-containing polymer composition can continuously release the therapeutic agent at a set or predetermined drug delivery rate to achieve desirable therapeutic results such as minimizing and/or reducing LSA.

In certain embodiments, the therapeutic agent-containing polymer composition can continuously release the therapeutic agent at a drug delivery rate (e.g., an average drug delivery rate) of about 0.001 μg/day to about 1 mg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.001 μg/day to about 5 μg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.001 μg/day to about 1 μg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.001 μg/day to about 0.1 μg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.01 μg/day to about 100 μg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.01 μg/day to about 10 μg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.01 μg/day to about 1 mg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of less than about 1 mg/day of the therapeutic agent or less than about 0.5 mg/day of the therapeutic agent, e.g., dexamethasone.

In certain embodiments, the therapeutic agent-containing polymer composition can continuously release the therapeutic agent at a drug delivery rate (e.g., an average drug delivery rate) of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the therapeutic agent, e.g., dexamethasone, or any values in between. In certain embodiments, the therapeutic agent-containing polymer composition can continuously release the therapeutic agent at a drug delivery rate (e.g., an average drug delivery rate) of about 0.2 μg/day to about 5 μg/day of the therapeutic agent, e.g., dexamethasone. In certain embodiments, the therapeutic agent-containing polymer composition can continuously release the therapeutic agent at a drug delivery rate (e.g., an average drug delivery rate) of about 0.2 μg/day to about 2 μg/day of the therapeutic agent, e.g., dexamethasone. In certain embodiments, the therapeutic agent-containing polymer composition can continuously release the therapeutic agent at a drug delivery rate (e.g., an average drug delivery rate) of about 0.2 μg/day to about 1 μg/day of the therapeutic agent, e.g., dexamethasone. In certain embodiments, the therapeutic agent-containing polymer composition can continuously release the therapeutic agent at a set or predetermined drug delivery rate to achieve desirable therapeutic results such as reducing, minimizing, preventing, and/or inhibiting the inflammation and/or infection.

In certain embodiments, the therapeutic agent-containing polymer composition can continuously release the therapeutic agent at a set or predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days. In certain embodiments, the therapeutic agent-containing polymer composition can continuously release the therapeutic agent at a set or predetermined drug delivery rate for a set or predetermined days such as for at least 20 days. In certain embodiments, the therapeutic agent-containing polymer composition can continuously release the therapeutic agent at a set or predetermined drug delivery rate for a set or predetermined days such as for at least 25 days. In certain embodiments, the therapeutic agent-containing polymer composition can continuously release the therapeutic agent at a set or predetermined drug delivery rate for a set or predetermined days such as for at least 30 days.

In certain embodiments, a therapeutic agent-containing polymer composition of the present disclosure releases about 1% to about 100% of the therapeutic agent present within the composition (e.g., of the total amount of therapeutic agent loaded into the composition), e.g., within a period of about 30-31 days (e.g., when implanted in a subject).

In certain embodiments, a therapeutic agent-containing polymer composition of the present disclosure releases about 30% to about 100% of the therapeutic agent present within the composition (e.g., of the total amount of therapeutic agent loaded into the composition), e.g., within a period of about 30-31 days. In certain embodiments, a therapeutic agent-containing polymer composition of the present disclosure releases about 50% to about 100% of the therapeutic agent present within the composition (e.g., of the total amount of therapeutic agent loaded into the composition), e.g., within a period of about 30-31 days. In certain embodiments, a therapeutic agent-containing polymer composition of the present disclosure releases about 30% to about 90% of the therapeutic agent present within the composition (e.g., of the total amount of therapeutic agent loaded into the composition), e.g., within a period of about 30-31 days. In certain embodiments, a therapeutic agent-containing polymer composition of the present disclosure releases about 30% to about 80% of the therapeutic agent present within the composition (e.g., of the total amount of therapeutic agent loaded into the composition), e.g., within a period of about 30-31 days. In certain embodiments, a therapeutic agent-containing polymer composition of the present disclosure releases about 30% to about 80% of the therapeutic agent present within the composition (e.g., of the total amount of therapeutic agent loaded into the composition), e.g., within a period of about 30-31 days. In certain embodiments, a period of about 30-31 days is the wear time of a sensor described herein. In certain embodiments, a period of about 30-31 days is the life span of a sensor described herein.

In certain embodiments, no more than about 90% of therapeutic agent present within the therapeutic agent-containing polymer composition (e.g., of the total amount of therapeutic agent loaded into the composition) is released, e.g., within a period of about 30-31 days. In certain embodiments, no more than about 70% of therapeutic agent present within the therapeutic agent-containing polymer composition (e.g., of the total amount of therapeutic agent loaded into the composition) is released, e.g., within a period of about 30-31 days.

In certain embodiments, no more than about 30%, 35%, 40%, 45%, 50%, 55%, 60%, 65%, 70% or 75% of the therapeutic agent present within the therapeutic agent-containing polymer composition (e.g., of the total amount of therapeutic agent loaded into the composition) is released in the first 5, 6 or 7 days after insertion of the composition.

In certain embodiments, no more than about 30% of the therapeutic agent present within the therapeutic agent-containing polymer composition (e.g., of the total amount of therapeutic agent loaded into the composition) is released in the first 5, 6 or 7 days after insertion of the composition. In certain embodiments, no more than about 40% of the therapeutic agent present within the therapeutic agent-containing polymer composition (e.g., of the total amount of therapeutic agent loaded into the composition) is released in the first 5, 6 or 7 days after insertion of the composition. In certain embodiments, no more than about 50% of the therapeutic agent present within the therapeutic agent-containing polymer composition (e.g., of the total amount of therapeutic agent loaded into the composition) is released in the first 5, 6 or 7 days after insertion of the composition. In certain embodiments, no more than about 60% of the therapeutic agent present within the therapeutic agent-containing polymer composition (e.g., of the total amount of therapeutic agent loaded into the composition) is released in the first 5, 6 or 7 days after insertion of the composition. In certain embodiments, no more than about 70% of the therapeutic agent present within the therapeutic agent-containing polymer composition (e.g., of the total amount of therapeutic agent loaded into the composition) is released in the first 5, 6 or 7 days after insertion of the composition.

In certain embodiments, a therapeutic agent-containing polymer composition of the present disclosure includes one or more polymers. Non-limiting examples of polymers for use in a therapeutic agent-containing polymer composition of the present disclosure are disclosed in U.S. 2003/0042137, PCT/US2022/011058 and PCT/US2024/010088, the contents of each of which are incorporated herein in their entireties. For example, but not by way of limitation, pages 82-84, 102-104 and 112 of PCT/US2022/011058 and pages 22-32 of PCT/US2024/010088 disclose polymers (e.g., copolymers) that can be used in a therapeutic agent-containing polymer composition of the present disclosure.

In certain embodiments, the polymer of the therapeutic agent-containing polymer composition can include a polymer selected from a polyvinylpyridine-based polymer, a polyvinylimidazole-based polymer, a polyacrylate-based polymer, a polyurethane-based polymer, a polyether urethane-based polymer, a silicone-based polymer, a derivative thereof, copolymers thereof and combinations thereof.

In certain embodiments, the polymer of the therapeutic agent-containing polymer composition comprises acrylate.

In certain embodiments, the polymer of the therapeutic agent-containing polymer composition is a polyurethane polymer comprising acrylate.

In certain embodiments, the polymer of the therapeutic agent-containing polymer composition is a polyacrylate-based polymer.

In certain embodiments, the polymer of the therapeutic agent-containing polymer composition is a polyacrylate-based polymer.

In certain embodiments, the polymer of the therapeutic agent-containing polymer composition is a poly(2-hydroxyethyl methacrylate).

In certain embodiments, the polymer of the therapeutic agent-containing polymer composition is a polyvinylpyridine-based polymer.

In certain embodiments, the polymer of the therapeutic agent-containing polymer composition is a polyurethane-based polymer.

In certain embodiments, the polymer of the therapeutic agent-containing polymer composition is a polyurethane polyurea polymer.

In certain embodiments, the polymer of the therapeutic agent-containing polymer composition is a polyurethane-based polymer comprising silicone.

In certain embodiments, the polymer of the therapeutic agent-containing polymer composition is a polymer comprising polyurethane block polymers and polysiloxane.

In certain embodiments, the polymer of the therapeutic agent-containing polymer composition can include a copolymer selected from a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and combinations thereof.

In certain embodiments, the polymer of the therapeutic agent-containing polymer composition can include a copolymer selected from a polyvinylpyridine-based copolymer, a polyvinylimidazole-based copolymer, and a combination thereof.

In certain embodiments, the polyvinylimidazole-based copolymer can be a polyvinylimidazole-co-polystyrene polymer. In certain embodiments, the polyvinylimidazole-co-polystyrene polymer can be a poly(N-vinylimidazole)-co-polystyrene polymer, a poly(1-vinylimidazole)-co-polystyrene polymer, or a derivative thereof.

In certain embodiments, the polyvinylpyridine-based copolymer can be a copolymer of vinylpyridine and styrene or a derivative thereof.

In certain embodiments, the polymer of the therapeutic agent-containing polymer composition can include a polyvinylpyridine-co-polystyrene polymer.

In certain embodiments, the polyvinylpyridine-co-polystyrene polymer can be a poly(4-vinylpyridine)-co-poly styrene polymer, a poly(2-vinylpyridine)-co-poly styrene polymer, or a derivative thereof.

In certain embodiments, the polymer of the therapeutic agent-containing polymer composition can include poly(4-vinylpyridine-co-styrene).

In certain embodiments, the polyvinylpyridine-co-polystyrene polymer can include about 1-50 mer % of styrene units, about 1-40 mer % of styrene units, about 1-30 mer % of styrene units, about 1-20 mer % of styrene units, or about 1-15 mer % of styrene units. In certain embodiments, the polyvinylpyridine-co-polystyrene polymer can include 1-50 mer % of styrene units. In certain embodiments, the polyvinylpyridine-co-polystyrene polymer can include 10-50 mer % of styrene units. In certain embodiments, the polyvinylpyridine-co-polystyrene polymer can include 10-40 mer % of styrene units. In certain embodiments, the polyvinylpyridine-co-polystyrene polymer can include 10-30 mer % of styrene units. In certain embodiments, the polyvinylpyridine-co-polystyrene polymer can include about 1-40 mer % of styrene units. In certain embodiments, the polyvinylpyridine-co-polystyrene polymer can include about 1-30 mer % of styrene units.

In certain embodiments, the polyvinylpyridine-co-polystyrene polymer can include about 1-20 mer % of styrene units. In certain embodiments, the polyvinylpyridine-co-polystyrene polymer can include about 1-15 mer % of styrene units.

In certain embodiments, the polyvinylpyridine-co-polystyrene polymer can include about 5-25 mer % of styrene units. In certain embodiments, the polyvinylpyridine-co-polystyrene polymer can include about 5-20 mer % of styrene units.

In certain embodiments, the polyvinylpyridine-co-polystyrene polymer can include about 5-15 mer % of styrene units.

In certain embodiments, the polymer of the therapeutic agent-containing polymer composition can be a biodegradable or bioresorbable polymer, such as but not limited to polycaprolactone (PCL), poly(glycolic acid) (PGA), poly(lactic acid) (PLA) or poly(D,L-lactide-co-glycolide) (PLGA). In certain embodiments, the polymer can be a polylactide, a polyglycolide or polyethylene glycol polymer. In certain embodiments, the polymer can be a blend of two or three of these functionalities as a block copolymer, e.g., a diblock copolymer or a triblock copolymer. Non-limiting embodiments of such block copolymers include poly(D,L-lactic-co-glycolic acid) (PLGA) and triblock copolymer polylactide-block-poly(ethylene glycol)-block-polylactide (PLA-PEG-PLA). Additional non-limiting examples of block copolymers include PEO and PPO copolymers such as PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof.

In certain embodiments, a weight average molecular weight of the polymer (e.g., copolymer) is in a range of about 5 kD-1,000 kD, about 5 kD-900 kD, about 5 kD-800 kD, about 5 kD-700 kD, about 5 kD-600 kD, about 5 kD-500 kD, about 5 kD-400 kD, about 5 kD-300 kD, about 10 kD-300 kD, about 20 kD-300 kD, about 30 kD-300 kD, about 40 kD-300 kD, about 50 kD-300 kD, about 60 kD-300 kD, about 70 kD-300 kD, about 80 kD-300 kD, about 90 kD-300 kD, about 100 kD-300 kD, or about 100 kD-200 kD, or with any range defined between any two of the foregoing values, such as in a range of about 100 kD-400 kD. In certain embodiments, a weight average molecular weight of the polymer (e.g., copolymer) is in a range of about 100 kD-250 kD. In certain embodiments, the molecular weight of a polymer (e.g., copolymer) can be determined by a suitable method such as gel permeation chromatography.

In certain embodiments, the polymer (e.g., copolymer) for use in the present disclosure is capable of absorbing from about 5% to about 95% of its weight in water. For example, but not by way of limitation, the polymer (e.g., copolymer) for use in the present disclosure is capable of absorbing from about 5% to about 95%, from about 5% to about 90%, from about 5% to about 85%, from about 10% to about 95%, from about 15% to about 95%, from about 20% to about 95%, from about 25% to about 95%, from about 30% to about 95%, from about 5% to about 30%, from about 5% to about 35%, from about 5% to about 25% or from about 5% to about 20%. In certain embodiments, the polymer (e.g., copolymer) is capable of absorbing at least about 5% of its weight in water. In certain embodiments, the polymer (e.g., copolymer) is capable of absorbing at least about 10% of its weight in water. In certain embodiments, the polymer (e.g., copolymer) is capable of absorbing at least about 20% of its weight in water. In certain embodiments, the polymer (e.g., copolymer) is capable of absorbing at least about 30% of its weight in water. In certain embodiments, the polymer (e.g., copolymer) is capable of absorbing at least about 40% of its weight in water. In certain embodiments, the polymer (e.g., copolymer) is capable of absorbing at least about 50% of its weight in water. In certain embodiments, the polymer (e.g., copolymer) is capable of absorbing at least about 60% of its weight in water. In certain embodiments, the polymer (e.g., copolymer) is capable of absorbing at least about 70% of its weight in water. In certain embodiments, the polymer (e.g., copolymer) is capable of absorbing at least about 80% of its weight in water. In certain embodiments, the polymer (e.g., copolymer) is capable of absorbing at least about 90% of its weight in water. In certain embodiments, the polymer (e.g., copolymer) is capable of absorbing at least about 95% of its weight in water. In certain embodiments, the polymer (e.g., copolymer) is capable of absorbing from about 5% to about 25% of its weight in water.

In certain embodiments, the polymer (e.g., copolymer) for use in the present disclosure can have a hardness from about 20 to about 100 Shore A. For example, but not by way of limitation, the polymer (e.g., copolymer) for use in the present disclosure can have a hardness from about 20 to about 90 Shore A, about 20 to about 80 Shore A, about 20 to about 70 Shore A, about 20 to about 60 Shore A, about 20 to about 50 Shore A, about 20 to about 40 Shore A, about 20 to about 30 Shore A, about 30 to about 100 Shore A, about 40 to about 100 Shore A, about 50 to about 100 Shore A, about 60 to about 100 Shore A, about 70 to about 100 Shore A, about 80 to about 100 Shore A, about 90 to about 100 Shore A, about 70 to about 95 Shore A, about 70 to about 90 Shore A, from about 70 to about 85 Shore A, from about 70 to about 80 Shore A, from about 75 to about 95 Shore A, from about 80 to about 95 Shore A, from about 85 to about 95 Shore A, from about 80 to about 93 Shore A or from about 80 to about 90 Shore A. In certain embodiments, the polymer (e.g., copolymer) for use in the present disclosure can have a hardness of about 80 Shore A. In certain embodiments, the polymer (e.g., copolymer) for use in the present disclosure can have a hardness of about 90 Shore A. In certain embodiments, the polymer (e.g., copolymer) for use in the present disclosure can have a hardness of about 93 Shore A. In certain embodiments, the polymer (e.g., copolymer) for use in the present disclosure can have a hardness of about 80 to about 100 Shore A, e.g., prior to implantation in a subject or prior to being hydrated. In certain embodiments, the polymer (e.g., copolymer) for use in the present disclosure can have a hardness of about 20 to about 60 Shore A, e.g., when implanted in a subject or when hydrated.

In certain embodiments, the linear expansion of a polymer (e.g., copolymer) for use in the present disclosure is from about 10% to about 200%. For example, but not by way of limitation, the linear expansion of a polymer (e.g., copolymer) for use in the present disclosure can be from about 10% to about 190%, from about 10% to about 180%, from about 10% to about 170%, from about 10% to about 160%, from about 10% to about 150%, from about 10% to about 140%, from about 10% to about 130%, from about 10% to about 120%, from about 10% to about 110%, from about 10% to about 100%, from about 25% to about 100%, from about 30% to about 100%, from about 35% to about 100%, from about 40% to about 100%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%, from about 20% to about 95%, from about 20% to about 90%, from about 20% to about 85%, from about 20% to about 80%, from about 20% to about 75%, from about 20% to about 70%, from about 20% to about 65%, from about 20% to about 60%, from about 20% to about 55%, from about 20% to about 50%, from about 20% to about 45%, from about 20% to about 40%, from about 20% to about 30%, from about 30% to about 60%, from about 40% to about 50%, from about 40% to about 60%, from about 20% to about 30% or from about 50% to about 70%. In certain embodiments, the linear expansion of a polymer (e.g., copolymer) for use in the present disclosure is about 25%. In certain embodiments, the linear expansion of a polymer (e.g., copolymer) for use in the present disclosure is about 40%. In certain embodiments, the linear expansion of a polymer (e.g., copolymer) for use in the present disclosure is about 45%. In certain embodiments, the linear expansion of a polymer (e.g., copolymer) for use in the present disclosure is about 50%. In certain embodiments, the linear expansion of a polymer (e.g., copolymer) for use in the present disclosure is about 60%. In certain embodiments, the linear expansion of a polymer (e.g., copolymer) for use in the present disclosure is of about 100%. In certain embodiments, the linear expansion of a polymer (e.g., copolymer) for use in the present disclosure is about 110% or greater, of about 100% or greater, of about 90% or greater, of about 80% or greater, of about 70% or greater, of about 60% or greater, of about 50% or greater, of about 40% or greater, of about 30% or greater, of about 20% or greater or of about 10% or greater. In certain embodiments, the linear expansion of a polymer (e.g., copolymer) for use in the present disclosure is about 110% or greater. In certain embodiments, the linear expansion of a polymer (e.g., copolymer) for use in the present disclosure is about 100% or greater. In certain embodiments, the linear expansion of a polymer (e.g., copolymer) for use in the present disclosure is about 90% or greater. In certain embodiments, the linear expansion of a polymer (e.g., copolymer) for use in the present disclosure is about 80% or greater. In certain embodiments, the linear expansion of a polymer (e.g., copolymer) for use in the present disclosure is about 70% or greater. In certain embodiments, the linear expansion of a polymer (e.g., copolymer) for use in the present disclosure is about 60% or greater. In certain embodiments, the linear expansion of a polymer (e.g., copolymer) for use in the present disclosure is about 50% or greater. In certain embodiments, the linear expansion of a polymer (e.g., copolymer) for use in the present disclosure is about 40% or greater. In certain embodiments, the linear expansion of a polymer (e.g., copolymer) for use in the present disclosure is about 30% or greater. In certain embodiments, the linear expansion of a polymer (e.g., copolymer) for use in the present disclosure is about 20% or greater. In certain embodiments, the linear expansion of a polymer (e.g., copolymer) for use in the present disclosure is about 10% or greater.

In certain embodiments, the polymer (e.g., copolymer) has a linear expansion of about 45% and is capable of absorbing about 70% of its weight in water. In certain embodiments, the polymer (e.g., copolymer) has a linear expansion of about 25% and is capable of absorbing about 55% of its weight in water. In certain embodiments, the polymer (e.g., copolymer) has a linear expansion of about 40% and is capable of absorbing about 60% of its weight in water. In certain embodiments, the polymer (e.g., copolymer) has a linear expansion of about 50% and is capable of absorbing about 50% of its weight in water. In certain embodiments, the polymer (e.g., copolymer) has a linear expansion of about 60% and is capable of absorbing about 80% of its weight in water. In certain embodiments, the polymer (e.g., copolymer) has a linear expansion of about 100% and is capable of absorbing about 90% of its weight in water. In certain embodiments, the polymer (e.g., copolymer) has a linear expansion of about 10% and is capable of absorbing about 30% of its weight in water. In certain embodiments, the polymer (e.g., copolymer) has a linear expansion of about 180% and is capable of absorbing about 95% of its weight in water.

In certain embodiments, a therapeutic agent-containing polymer composition of the present disclosure can further include a crosslinker. In certain embodiments, the polyvinylpyridine-co-polystyrene polymer can be further crosslinked with a suitable crosslinking agent. In certain embodiments, the crosslinker can be a diglycidyl- or triglycidyl-functional epoxy.

In certain embodiments, the crosslinking agent can include polyepoxides, carbodiimide, cyanuric chloride, triglycidyl glycerol (Gly3), N-hydroxysuccinimide, imidoesters, epichlorohydrin, or derivatized variants thereof. In certain embodiments, the crosslinking agent is polyethylene glycol diglycidyl ether.

In certain embodiments, the crosslinker can be selected from the group consisting of diglycidyl-PEG (200-1000), glycerol triglycidyl ether, and a combination thereof. For example, but not by way of limitation, the crosslinker is a diglycidyl-PEG (200-1000) with a molecular weight of 200 g/mol-1000 g/mol. The term “diglycidyl-PEG” utilized in the present disclosure can refer to polyethylene glycol diglycidyl ether.

In certain embodiments, the crosslinker can be selected from the group consisting of diglycidyl-PEG 200, diglycidyl-PEG 400, glycerol triglycidyl ether, and a combination thereof. In certain embodiments, the crosslinker can be diglycidyl-PEG 200. In certain embodiments, the crosslinker can be diglycidyl-PEG 400. In certain embodiments, the crosslinker can be glycerol triglycidyl ether.

In certain embodiments, a therapeutic agent-containing polymer composition comprises a crosslinker (e.g., includes a certain amount of crosslinker) that provides a certain mol % of crosslinking of the polymer, e.g., the copolymer, present within the therapeutic agent-containing polymer composition. In certain embodiments, mol % crosslinking of the polymer, e.g., the copolymer, can be in a range of about 0.1 mol %-50 mol %. In certain embodiments, mol % crosslinking of the polymer, e.g., the copolymer, can be in a range of about 1 mol %-50 mol %. In certain embodiments, mol % crosslinking of the polymer, e.g., the copolymer, can be in a range of about 0.1 mol %-40 mol %. In certain embodiments, mol % crosslinking of the polymer, e.g., the copolymer, can be in a range of about 1 mol %-40 mol %. In certain embodiments, mol % crosslinking of the polymer, e.g., the copolymer, can be in a range of about 0.1 mol %-30 mol %. In certain embodiments, mol % crosslinking of the copolymer can be in a range of about 0.2 mol %-30 mol %. In certain embodiments, mol % crosslinking of the polymer, e.g., the copolymer, can be in a range of about 1 mol %-30 mol %. In certain embodiments, mol % crosslinking of the polymer, e.g., the copolymer, can be in a range of about 0.1 mol %-25 mol %. In certain embodiments, mol % crosslinking of the polymer, e.g., the copolymer, can be in a range of about 1 mol %-25 mol %. In certain embodiments, mol % crosslinking of the polymer, e.g., the copolymer, can be in a range of about 0.1 mol %-20 mol %. In certain embodiments, mol % crosslinking of the polymer, e.g., the copolymer, can be in a range of about 1 mol %-20 mol %. In certain embodiments, mol % crosslinking of the polymer, e.g., the copolymer, can be in a range of about 0.1 mol %-15 mol %. In certain embodiments, mol % crosslinking of the polymer, e.g., the copolymer, can be in a range of about 1 mol %-15 mol %. In certain embodiments, mol % crosslinking of the polymer, e.g., the copolymer, can be in a range of about 0.1 mol %-10 mol %. In certain embodiments, mol % crosslinking of the polymer, e.g., the copolymer, can be in a range of about 0.5 mol %-10 mol %. In certain embodiments, mol % crosslinking of the polymer, e.g., the copolymer, can be in a range of about 1 mol %-10 mol %.

In certain embodiments, a therapeutic agent-containing polymer composition of the present disclosure includes a crosslinker, where the amount of crosslinker in the therapeutic agent-containing polymer composition provides no more than about 20 mol % crosslinking (e.g., of the copolymer).

In certain embodiments, mol % crosslinking of the polymer can be in a range of about 0.5 mol %-10 mol %.

In certain embodiments, mol % crosslinking of the polymer can be in a range of about 1 mol %-10 mol %.

In certain embodiments, mol % crosslinking of the polymer can be in a range of about 1 mol %-25 mol %.

In certain embodiments, the therapeutic agent-containing polymer composition can include an amount of the crosslinker in a range of about 0.01 wt %-50 wt % based on the total weight of the polymer, e.g., the copolymer (e.g., in the therapeutic agent-containing polymer composition). In certain embodiments, the therapeutic agent-containing polymer composition can include an amount of the crosslinker in a range of about 0.01 wt %-40 wt % based on the total weight of the polymer, e.g., the copolymer. In certain embodiments, the therapeutic agent-containing polymer composition can include an amount of the crosslinker in a range of about 0.01 wt %-30 wt % based on the total weight of the polymer, e.g., the copolymer. In certain embodiments, the therapeutic agent-containing polymer composition can include an amount of the crosslinker in a range of about 0.01 wt %-20 wt % based on the total weight of the polymer, e.g., the polymer, e.g., the copolymer. In certain embodiments, the therapeutic agent-containing polymer composition can include an amount of the crosslinker in a range of about 0.01 wt %-15 wt % based on the total weight of the polymer, e.g., the copolymer. In certain embodiments, the therapeutic agent-containing polymer composition can include an amount of the crosslinker in a range of about 0.01 wt %-10 wt % based on the total weight of the polymer, e.g., the copolymer. In certain embodiments, the therapeutic agent-containing polymer composition can include an amount of the crosslinker in a range of about 1 wt %-15 wt % based on the total weight of the polymer, e.g., the copolymer. In certain embodiments, the therapeutic agent-containing polymer composition can include an amount of the crosslinker in a range of about 1 wt %-10 wt % based on the total weight of the polymer, e.g., the copolymer. In certain embodiments, the therapeutic agent-containing polymer composition can include an amount of the crosslinker in a range of about 1 wt %-5 wt % based on the total weight of the polymer, e.g., the copolymer.

In certain embodiments, the crosslinker is triglycidyl glycerol (Gly3) and the mol % crosslinking of the polymer can be in a range of about 1 mol %-15 mol %.

In certain embodiments, the therapeutic agent-containing polymer composition includes (i) a polyvinylpyridine-co-polystyrene copolymer comprising about 5-15 mer % of styrene units and (ii) a crosslinker, wherein the mol % crosslinking of the copolymer can be in a range of about 1 mol %-15 mol %.

In certain embodiments, the therapeutic agent-containing polymer composition includes (i) a polyvinylpyridine-co-polystyrene copolymer comprising about 5-15 mer % of styrene units and (ii) a triglycidyl glycerol (Gly3) crosslinker, wherein the mol % crosslinking of the copolymer can be in a range of about 1 mol %-15 mol %.

In certain embodiments, the therapeutic agent can be incorporated into the polymer of the therapeutic agent-containing polymer composition. For example, but not way of limitation, the therapeutic agent can be non-covalently mixed with and/or entrapped within the polymer of the therapeutic agent-containing polymer composition.

In certain embodiments, the therapeutic agent can be non-covalently mixed with and/or entrapped within a therapeutic agent-containing polymer composition comprising a polyvinylpyridine-co-polystyrene copolymer.

In certain embodiments, the therapeutic agent can be coupled to (e.g., covalently attached to) a polymer of the therapeutic agent-containing polymer composition. In certain embodiments, the therapeutic agent can be coupled (e.g., covalently attached) directly or via a linker to one or more chains and/or monomers of the polymer.

In certain embodiments, the therapeutic agent can be reversibly coupled to a therapeutic agent such that the therapeutic agent is uncoupled from the polymer under certain conditions. In certain embodiments, the therapeutic agent can be uncoupled from the polymer in response to the environmental conditions once inserted into a subject. In certain embodiments, the therapeutic agent can be uncoupled from the polymer upon exposure to aqueous media (e.g., interstitial fluid). In certain embodiments, the therapeutic agent can be uncoupled from the polymer upon exposure to glucose. In certain embodiments, the therapeutic agent can be uncoupled from the polymer in response to an alteration in the pH of the environment in which the analyte sensor is inserted. In certain embodiments, the therapeutic agent can be uncoupled from the polymer in response to an alteration in the temperature of the environment in which the analyte sensor is inserted. In certain embodiments, the therapeutic agent can be uncoupled from the polymer in response to an electrochemical stimulus, e.g., from a voltage applied to the analyte sensor and/or from a current within the analyte sensor. In certain embodiments, the therapeutic agent can be uncoupled from polymer by an enzyme, an active oxygen species such as hydrogen peroxide, temperature, ions, pH and the like.

In certain embodiments, the therapeutic agent can be coupled to (e.g., covalently attached to) one or more chains and/or monomers of the polymer via a hydrolyzable bond to allow delayed release of the therapeutic agent after insertion of the analyte sensor in vivo.

In certain embodiments, the therapeutic agent can be coupled to (e.g., covalently attached to) a polyvinylpyridine polymer present within the therapeutic agent-containing polymer composition via a hydrolyzable bond to allow delayed release of the therapeutic agent after insertion of the analyte sensor in vivo.

In certain embodiments, the therapeutic agent can be coupled to (e.g., covalently attached to) a poly(2-hydroxyethyl methacrylate) polymer present within the therapeutic agent-containing polymer composition. In certain embodiments, the therapeutic agent can be coupled to (e.g., covalently attached to) a poly(2-hydroxyethyl methacrylate) polymer present within the therapeutic agent-containing polymer composition via an acrylate moiety to allow delayed release of the therapeutic agent after insertion of the analyte sensor in vivo. In certain embodiments, the therapeutic agent can be uncoupled from the acrylate moiety as described above, e.g., upon exposure to aqueous media (e.g., interstitial fluid), upon exposure to glucose, in response to an alteration in the pH of the environment in which the analyte sensor is inserted, in response to an alteration in the temperature of the environment in which the analyte sensor is inserted and/or in response to an electrochemical stimulus, e.g., from a voltage applied to the analyte sensor and/or from a current within the analyte sensor.

### V. Incorporation of Therapeutic Agents into Analyte Sensors

The present disclosure further provides analyte sensors that include one or more therapeutic agents. For example, but not by way of limitation, the present disclosure provides analyte sensors that include one or more therapeutic agent-containing polymer compositions.

In certain embodiments, a therapeutic agent-containing polymer composition applied to an analyte sensor can be referred to as a drug-eluting membrane or a second polymer membrane herein.

The incorporation of a therapeutic agent within the analyte sensor itself allows for targeted delivery of the therapeutic agent to the tissue surrounding the implantation site and the analyte sensor and allows for the release of the therapeutic agent in close proximity to the analyte sensor in vivo. In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to prevent and/or reduce the late sensor attenuation (LSA). In certain embodiments, the therapeutic agent to be delivered according to the present disclosure can be a therapeutic agent that is effective at reducing, minimizing, preventing and/or inhibiting a tissue's response to analyte sensor implantation and/or a tissue infection, thus, to prevent and/or reduce analyte signal inaccuracy towards the end of sensor life.

Non-limiting examples of therapeutic agents that can be incorporated into an analyte sensor of the present disclosure are disclosed herein in Section II. In certain embodiments, the therapeutic agent can be an antibiotic agent, an antiviral agent, an anti-inflammatory agent, an anti-cancer agent, an antiplatelet agent, an anticoagulant agent, a coagulant agent, an antiglycolytic agent and combinations thereof. In certain embodiments, the anti-inflammatory agent can be a non-steroidal anti-inflammatory agent. In certain embodiments, the anti-inflammatory agent can be a steroidal anti-inflammatory agent, e.g., a corticosteroid. Non-limiting examples of anti-inflammatory agents can include triamcinolone, betamethasone, dexamethasone, hydrocortisone, prednisone, methylprednisolone, fludrocortisone, acetylsalicylic acid, isobutylphenylpropanoic acid or a derivative thereof, a salt thereof or a prodrug thereof. Non-limiting salt forms can include pharmaceutically acceptable salts including acetate and phosphate salts. In certain embodiments, the anti-inflammatory agent can be a salt of dexamethasone. In certain embodiments, the anti-inflammatory agent can be a derivative of dexamethasone. In certain embodiments, the dexamethasone derivative or salt form can be dexamethasone acetate. In certain embodiments, the dexamethasone derivative or salt form can be dexamethasone sodium phosphate.

Non-limiting examples of analyte sensors that can be modified to incorporate a therapeutic agent (e.g., in a therapeutic agent-containing polymer composition) are disclosed in Section III. In certain embodiments, the analyte sensor is a dermal sensor. In certain embodiments, the analyte sensor is a subcutaneous sensor such as a subcutaneously implanted sensor. In certain embodiments, the analyte sensor is an intravenous sensor such as intravenously implanted sensor. In certain embodiments, the analyte sensor is configured to detect glucose. In certain embodiments, the analyte sensor is configured to detect glucose and ketones. In certain embodiments, the analyte sensor is configured to detect lactate. In certain embodiments, the analyte sensor is configured to detect creatinine. In certain embodiments, the analyte sensor is configured to detect alcohol. In certain embodiments, the analyte sensor has a second working electrode, e.g., to detect a second analyte, as described in Section III.

The present disclosure provides an analyte sensor of the present disclosure, e.g., a sensor tail (i.e., in vivo portion) 100 or 200, further comprising a therapeutic agent-containing polymer composition. FIGS. 1A-1B and 17A-17B illustrate a cross-sectional diagram of an exemplary analyte sensor according to certain embodiments of the present disclosure. As shown in FIG. 1A, the analyte sensor can include: (i) a sensor tail (i.e., in vivo portion) 200 including at least a first working electrode 214 on a substrate 212; (ii) an active area 218 disposed upon a surface of the first working electrode for detecting an analyte; (iii) a mass transport limiting membrane 220 permeable to the analyte that coats at least the active area; (iv) a counter/reference electrode 216 on the substrate 212; and (v) a therapeutic agent-containing polymer composition. As shown in FIG. 1B, the analyte sensor can include a sensor tail (i.e., in vivo portion) 100 including: (i) a substrate 102; (ii) a first working electrode 104 on the substrate 102; (ii) a sensing spot 108 disposed upon a surface of the first working electrode for detecting an analyte; (iii) a membrane 110 overcoating at least the sensing spot 108; (iv) a counter/reference electrode 106 on the substrate 102 and (v) a therapeutic agent-containing polymer composition. As shown in FIG. 2, the analyte sensor can include a sensor tail (i.e., in vivo portion) 100 including: (i) a substrate 102; (ii) a first working electrode 104 on the substrate 102; (ii) a plurality of sensing spots 108a-108f disposed upon a surface of the first working electrode for detecting an analyte; (iii) a membrane 110 overcoating at least the sensing spot 108; (iv) a counter/reference electrode 106 on the substrate 102 and (v) a therapeutic agent-containing polymer composition.

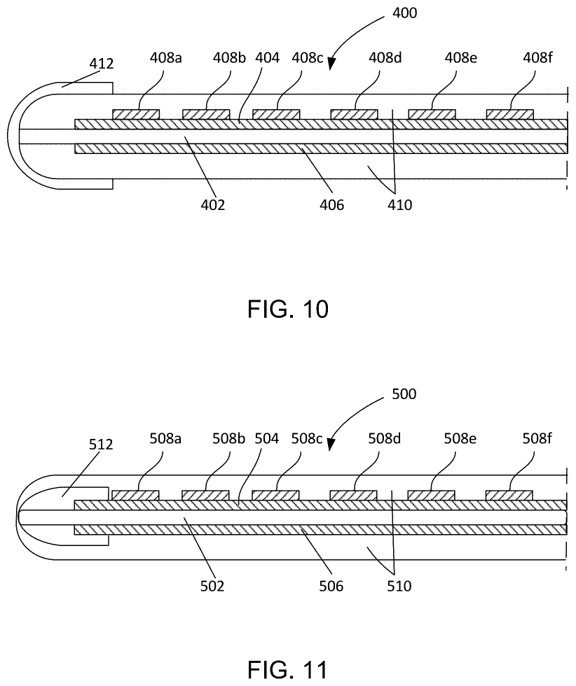

In certain embodiments, the therapeutic agent-containing polymer composition can be disposed upon a structure or component of the in vivo portion (i.e., sensor tail). In certain embodiments, the therapeutic agent-containing polymer composition can be disposed upon or incorporated into a component of the analyte sensor (e.g., an in vivo portion of the analyte sensor). For example, but not by way of limitation, the therapeutic agent-containing polymer composition can be disposed upon a surface of an electrode (e.g., a counter/reference electrode (e.g., 216 of FIG. 18A) and/or a working electrode (e.g., 214 of FIG. 18A)), an insulating material (e.g., a dielectric material (e.g., 219a-c of FIG. 18A)), a substrate (e.g., 212 of FIG. 18A), a mass transport limiting membrane (e.g., 220 of FIG. 18A) and a distal tip of the analyte sensor (e.g., 412 of FIG. 10 and 512 of FIG. 11). In certain embodiments, the therapeutic agent-containing polymer composition can be disposed upon a portion of the substrate and/or working electrode that surrounds the active area. For example, but not by way of limitation, the therapeutic agent-containing polymer composition can be deposited on non-sensing chemistry portions of the working electrode. In certain embodiments, the therapeutic agent-containing polymer composition can be deposited on non-sensing chemistry portions of the substrate. In certain embodiments, the therapeutic agent-containing polymer composition can be disposed upon a portion of the substrate and/or working electrode that is distal to the active area. In certain embodiments, the therapeutic agent-containing polymer composition can be disposed upon a portion of the substrate and/or working electrode that is proximal to the active area. In certain embodiments, the therapeutic agent-containing polymer composition can be disposed upon a portion of the substrate and/or working electrode that surrounds the active area.

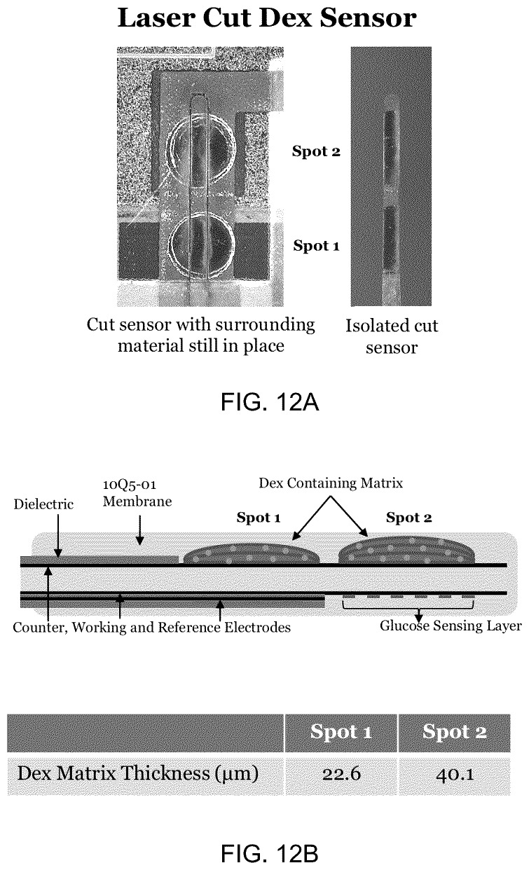

In certain embodiments, the therapeutic agent-containing polymer composition can be disposed upon the counter/reference electrode. In certain embodiments where the analyte sensor includes a counter electrode and a reference electrode, the therapeutic agent-containing polymer composition can be disposed on the counter/reference electrode. In certain embodiments, the therapeutic agent-containing polymer composition can be disposed on the counter electrode, e.g., as shown in FIG. 12A and FIG. 12B. In certain embodiments, the therapeutic agent-containing polymer composition can be disposed on the reference electrode. In certain embodiments where the analyte sensor includes a counter electrode and a reference electrode, the therapeutic agent-containing polymer composition can be disposed on the counter electrode. In certain embodiments where the analyte sensor includes a counter electrode and a reference electrode, the therapeutic agent-containing polymer composition can be disposed on the reference electrode, e.g., as shown in FIG. 12A and FIG. 12B.

In certain embodiments, the therapeutic agent-containing polymer composition can be disposed upon a structure of the in vivo portion (i.e., sensor tail) (e.g., the counter/reference electrode) as multiple discrete areas, e.g., as shown in FIG. 12A and FIG. 12B. In certain embodiments, the therapeutic agent-containing polymer composition can be disposed upon a structure of the in vivo portion (i.e., sensor tail) (e.g., the counter/reference electrode) as multiple spots, e.g., as shown in FIG. 12A and FIG. 12B. For example, but not by way of limitation, the therapeutic agent-containing polymer composition can be disposed upon the counter/reference electrode as 1 or more spots, 2 or more spots, 3 or more spots, 4 or more spots, 5 or more spots, 6 or more spots, 7 or more spots, 8 or more spots, 9 or more spots, 10 or more spots, 11 or more spots, 12 or more spots, 13 or more spots, 14 or more spots, 15 or more spots, 16 or more spots, 17 or more spots, 18 or more spots, 19 or more spots, 20 or more spots, 21 or more spots, 22 or more spots, 23 or more spots, 24 or more spots, 25 or more spots, 26 or more spots, 27 or more spots, 28 or more spots, 29 or more spots or 30 or more spots. For example, but not by way of limitation, the therapeutic agent-containing polymer composition can be disposed upon the counter/reference electrode (e.g., the counter electrode) as two or more spots as shown in FIG. 12A and FIG. 12B. In certain embodiments, the therapeutic agent-containing polymer composition can be disposed upon a portion of the substrate and/or working electrode that surrounds the active area as one or more spots, e.g., two or more spots. In certain embodiments, the therapeutic agent-containing polymer composition can be disposed upon a portion of the substrate and/or working electrode that surrounds the active area. For example, but not by way of limitation, the therapeutic agent-containing polymer composition can be deposited on non-sensing chemistry portions of the working electrode as one or more spots, e.g., two or more spots. In certain embodiments, the therapeutic agent-containing polymer composition can be deposited on non-sensing chemistry portions of the substrate as one or more spots, e.g., two or more spots. In certain embodiments, the therapeutic agent-containing polymer composition can be disposed upon a portion of the substrate and/or working electrode that is distal to the active area as one or more spots, e.g., two or more spots. In certain embodiments, the therapeutic agent-containing polymer composition can be disposed upon a portion of the substrate and/or working electrode that is proximal to the active area as one or more spots, e.g., two or more spots. In certain embodiments, the therapeutic agent-containing polymer composition can be disposed upon a portion of the substrate and/or working electrode that surrounds the active area as one or more spots, e.g., two or more spots.

In certain embodiments, the one or more spots, e.g., two or more spots, can have the shape of a circle, can have the shape of an oval, can have the shape of a regular polygon or can have the shape of an irregular polygon. In certain embodiments, each of the spots have the same or similar shapes. For example, but not by way of limitation, each of the spots can have a circular or rounded shape. In certain embodiments, at least two or more of the spots have different shapes, e.g., one of the spots can be circular or oval in shape and a different spot can have the shape of a regular or irregular polygon.

In certain embodiments, each spot contains the same or similar amount of the therapeutic agent. Alternatively, at least one of the spots contains a different amount of the therapeutic agent compared to one other spot. In certain embodiments, at least two or more of the spots have a difference in the amount of therapeutic agent of about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 105% or more, about 110% or more, about 115% or more, about 120% or more, about 125% or more, about 130% or more, about 135% or more, about 140% or more, about 145% or more, about 150% or more, about 155% or more, about 160% or more, about 165% or more, about 170% or more, about 180% or more, about 185% or more, about 190% or more, about 195% or more or 200% or more. In certain embodiments, at least two or more of the spots have a difference in the amount of therapeutic agent of about 50% or more. In certain embodiments, at least two or more of the spots have a difference in the amount of therapeutic agent of about 60% or more. In certain embodiments, at least two or more of the spots have a difference in the amount of therapeutic agent of about 70% or more. In certain embodiments, at least two or more of the spots have a difference in the amount of therapeutic agent of about 80% or more. In certain embodiments, at least two or more of the spots have a difference in the amount of therapeutic agent of about 90% or more. In certain embodiments, at least two or more of the spots have a difference in the amount of therapeutic agent of about 100% or more.

In certain embodiments, each spot has the same or similar thickness. Alternatively, at least two or more of the spots differ in thicknesses, e.g., as shown in FIG. 12A and FIG. 12B. In certain embodiments, at least two or more of the spots have a difference in thickness of about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 105% or more, about 110% or more, about 115% or more, about 120% or more, about 125% or more, about 130% or more, about 135% or more, about 140% or more, about 145% or more, about 150% or more, about 155% or more, about 160% or more, about 165% or more, about 170% or more, about 180% or more, about 185% or more, about 190% or more, about 195% or more or 200% or more. For example, but not by way of limitation, at least two or more of the spots have a difference in thickness of about 50% or more. In certain embodiments, at least two or more of the spots have a difference in thickness of about 60% or more. In certain embodiments, at least two or more of the spots have a difference in thickness of about 70% or more. In certain embodiments, at least two or more of the spots have a difference in thickness of about 80% or more. In certain embodiments, at least two or more of the spots have a difference in thickness of about 90% or more. In certain embodiments, at least two or more of the spots have a difference in thickness of about 100% or more.

In certain embodiments, an analyte sensor of the present disclosure can include (i) a sensor tail (i.e., an in vivo portion) including at least a first working electrode on a substrate; (ii) an active area disposed upon a surface of the first working electrode for detecting an analyte; (iii) a mass transport limiting membrane permeable to the analyte that coats at least the active area; (iv) a counter/reference electrode on the substrate; and (v) at least one spot (e.g., at least two spots) of a therapeutic agent-containing polymer composition on a structure of the sensor tail (i.e., in vivo portion). In certain embodiments, mass transport limiting membrane also at least partially covers the at least one spot (e.g., at least two spots) of a therapeutic agent-containing polymer composition.

In certain embodiments, an analyte sensor of the present disclosure can include (i) a sensor tail including at least a first working electrode on a substrate; (ii) an active area disposed upon a surface of the first working electrode for detecting an analyte; (iii) a mass transport limiting membrane permeable to the analyte that coats at least the active area; (iv) a counter/reference electrode on the substrate; and (v) at least one spot (e.g., at least two spots) of a therapeutic agent-containing polymer composition on a surface of the counter/reference electrode. In certain embodiments, mass transport limiting membrane also at least partially covers the at least one spot (e.g., at least two spots) of a therapeutic agent-containing polymer composition. In certain embodiments, the analyte sensor includes two or more spots on a surface of the counter/reference electrode, where at least two of the two or more spots differ in thickness, e.g., where at least two of the two or more spots differ in thickness by more than about 50% (e.g., by more than about 70%). In certain embodiments, the mass transport limiting membrane can be disposed upon the at least one spot (e.g., at least two spots) of the therapeutic agent-containing polymer composition that is on the surface (e.g., the Ag/AgCl material) of the counter/reference electrode.

In certain embodiments, the therapeutic agent-containing polymer composition can be located at the distal most region of the sensor tail (e.g., in vivo portion of an analyte sensor). For example, but not by way of limitation, the distal tip of an analyte sensor, e.g., the distal tip of an in vivo portion of an analyte sensor, is coated with a therapeutic agent-containing polymer composition.

In certain embodiments, the therapeutic agent-containing polymer composition can be located in a region between the tip of the sensor tail (e.g., 300 in FIG. 7) and the beginning of the sensing layer (e.g., the edge of the first sensing spot (e.g., spot 108a of FIG. 7)), e.g., above or below the mass transport limiting membrane. In certain embodiments, the region between the tip of the sensor tail and the beginning of the sensing layer has a length, e.g., referred to as d3 in FIG. 7. In certain embodiments, as shown in FIG. 7, an exemplary sensor tail 300 can include a first region between a tip of the sensor tail 300 and a edge of a first sensing spot 108a with a first length d3 and a second region extending from the edge of the first sensing spot 108a along a direction away from the tip of the sensor tail and including the plurality of sensing spots 108a-108f with a second length d4. The first region cannot include (e.g., excludes) any sensing spot and can be a non-sensing region, and the second region can be a sensing region (e.g., including the plurality of sensing spots 108a-108f). In certain embodiments, a therapeutic agent-containing polymer composition can be deposited, e.g., by dipping, onto the first region (e.g., without contacting the second region).

In certain embodiments, the region between the tip of the sensor tail and the beginning of the sensing layer can have a length (e.g., d3 in FIG. 7) in a range of about 0.1 mm to about 1 mm, e.g., about 0.2 mm to about 1 mm, about 0.3 mm to about 1 mm, about 0.4 mm to about 1 mm, about 0.5 mm to about 1 mm, about 0.6 mm to about 1 mm or about 0.7 mm to about 1 mm. In certain embodiments, the region between the tip of the sensor tail and the beginning of the sensing layer can have a length (e.g., d3 in FIG. 7) in a range of about 0.5 mm to about 1 mm. In certain embodiments, the region between the tip of the sensor tail and the beginning of the sensing layer can have a length (e.g., d3 in FIG. 7) in a range of about 0.5 mm to about 1 mm. In certain embodiments, the region between the tip of the sensor tail and the beginning of the sensing layer can have a length (e.g., d3 in FIG. 7) of about 0.1 mm, about 0.2 mm, about 0.3 mm, about 0.4 mm, about 0.5 mm, about 0.6 mm, about 0.7 mm, about 0.8 mm, about 0.9 mm or about 1 mm. In certain embodiments, the first length of the first region can be a length between the tip of the sensor tail and the edge of the first sensing spots 108a which is proximal to the tip of the sensor tail among the plurality of sensing spots. In certain embodiments, the first length of the first region can be in a range of about 0.1 mm to about 1 mm, for example, about 0.2 mm to about 1 mm, about 0.3 mm to about 1 mm, about 0.4 mm to about 1 mm, about 0.5 mm to about 1 mm, about 0.6 mm to about 1 mm, or about 0.7 mm to about 1 mm. In certain embodiments, the first length of the first region can be about 0.1 mm, about 0.2 mm, about 0.3 mm, about 0.4 mm, about 0.5 mm, about 0.6 mm, about 0.7 mm, about 0.8 mm, about 0.9 mm, or about 1 mm. However, embodiments of the present disclosure are not limited thereto.

In certain embodiments, the second region that includes the sensing layer (e.g., the plurality of sensing spots) has a length, e.g., referred to as d4 in FIG. 7. For example, but not by way of limitation, the second length of the second region can be a length of a sensing region including the sensing layer (e.g., the plurality of sensing spots). In certain embodiments, the second length of the second region can be in a range of about 1 mm to about 3 mm, e.g., about 1 mm to about 2.9 mm, about 1 mm to about 2.8 mm, about 1 mm to about 2.7 mm, about 1 mm to about 2.6 mm, about 1 mm to about 2.5 mm, about 1 mm to about 2.5 mm, about 1 mm to about 2.4 mm, about 1 mm to about 2.3 mm, about 1 mm to about 2.2 mm, about 1 mm to about 2.1 mm, or about 1 mm to about 2 mm. In certain embodiments, the second length of the second region can be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm or about 3 mm. However, embodiments of the present disclosure are not limited thereto.

In certain embodiments, the therapeutic agent-containing polymer composition can be disposed on the mass transport limiting membrane. In certain embodiments, the therapeutic agent-containing polymer composition can be disposed on a portion of the mass transport limiting membrane. For example, but not by way of limitation, the therapeutic agent-containing polymer composition 512 and 412 can be disposed on the mass transport limiting membrane 510 and 410 located at the distal tip of the sensor tail, e.g., as shown in FIGS. 9 and 10. In certain embodiments, the therapeutic agent-containing polymer composition can be disposed on the mass transport limiting membrane located at the distal most region of the sensor tail (i.e., in vivo portion), where the therapeutic agent-containing polymer composition is not located on top of and/or not covering the analyte sensing layer, e.g., as shown in FIGS. 9 and 10. In certain embodiments, the therapeutic agent-containing polymer composition can be present on the portion of the mass transport limiting membrane that is disposed upon the region of the sensor tail located between the tip of the sensor tail and the beginning of the sensing layer.

In certain embodiments, the therapeutic agent-containing polymer composition can be located underneath the mass transport limiting membrane. For example, but not by way of limitation, the therapeutic agent-containing polymer composition can be located at the distal tip of the sensor tail under the mass transport limiting membrane 510, e.g., as shown in FIG. 11. In certain embodiments, the therapeutic agent-containing polymer composition can be disposed underneath the mass transport limiting membrane located at the distal most region of the sensor tail (i.e., in vivo portion), where the therapeutic agent-containing polymer composition is not located on top of and/or not covering the analyte sensing layer, e.g., as shown in FIG. 11. In certain embodiments, the therapeutic agent-containing polymer composition can be disposed upon the substrate, e.g., 502 of FIG. 11, and/or disposed upon the substrate and a portion of the working electrode 504 adjacent to but not covering the analyte sensing layer, e.g., shown as 508a-508f in FIG. 11. In certain embodiments, the therapeutic agent-containing polymer composition can be present on the distal region of the sensor tail located between the tip of the sensor tail and the beginning of the sensing layer and beneath the mass transport limiting membrane.

In certain embodiments, an exemplary analyte sensor includes an in vivo portion configured to reside below a skin surface of a subject and in contact with interstitial fluid of the subject, wherein the in vivo portion comprises a second region comprising an active area and a first region distal to the second region. In certain embodiments, the first region and the second region are coated with a first polymer membrane and the first region and not the second region is coated with a second polymer membrane comprising a therapeutic agent.

In certain embodiments, an exemplary analyte sensor includes an in vivo portion configured to reside below a skin surface of a subject and in contact with interstitial fluid of the subject, wherein the in vivo portion comprises:
(i) a substrate comprising (a) a second region comprising an active area and (b) a first region distal to the second region;
(ii) a working electrode on the substrate comprising the active area;
(iii) a counter/reference electrode on the substrate;
(iv) a first polymer membrane coated on the first region and the second region including active area; and
(v) a second polymer membrane coated on the first polymer membrane in the first region and not in the second region, wherein the second polymer membrane includes a therapeutic agent.

In certain embodiments, an exemplary analyte sensor includes an in vivo portion configured to reside below a skin surface of a subject and in contact with interstitial fluid of the subject, wherein the in vivo portion comprises:
(i) a substrate comprising (a) a second region comprising an active area and (b) a first region distal to the second region;
(ii) a working electrode on the substrate comprising the active area;
(iii) a counter/reference electrode on the substrate;
(iv) a second polymer membrane coated on the first region and not the second region, wherein the second polymer membrane includes a therapeutic agent; and
(v) a first polymer membrane coated on the first region and the second region including active area and on top of the second polymer membrane.

FIG. 10 illustrates an exemplary sensor tail (i.e., in vivo portion) including a mass-limiting membrane and a membrane comprising a therapeutic agent according to one or more embodiments of the present disclosure. As shown in FIG. 10, according to one or more embodiments of the present disclosure, an analyte sensor can include a sensor tail 400 (i.e., in vivo portion) including:
(i) a substrate 402;
(ii) a working electrode 404;
(iii) a counter/reference electrode 406 on the substrate 402;
(iv) a plurality of sensing spots 408a-408f on a surface of the working electrode 404 including a first sensing spot 408a proximal to a tip of the sensor tail among the plurality of sensing spots 408a-408f;
(v) a first region between the tip of the sensor tail 400 and an edge of the first sensing spot 408a with a first length and not including any sensor spot;
(vi) a second region extending from the edge of the first sensing spot 408a along a distal direction away from the tip of the sensor tail and including the plurality of sensing spots 408a-408f with a second length;
(vii) a first polymer membrane 410 coated on the sensor tail 400 in the first region and the second region including the plurality of sensing spots 408a-408f; and
(viii) a second polymer membrane 412, differing from the first polymer membrane 410, coated on the first polymer membrane 410 in the first region, and not in the second region,
wherein the second polymer membrane 412 including a therapeutic agent.

In certain embodiments, the first length of the first region of the sensor tail 400 can be in a range of about 0.1 mm to about 1 mm, for example, about 0.2 mm to about 1 mm, about 0.3 mm to about 1 mm, about 0.4 mm to about 1 mm, about 0.5 mm to about 1 mm, about 0.6 mm to about 1 mm, or about 0.7 mm to about 1 mm. In certain embodiments, the first length of the first region can be about 0.1 mm, about 0.2 mm, about 0.3 mm, about 0.4 mm, about 0.5 mm, about 0.6 mm, about 0.7 mm, about 0.8 mm, about 0.9 mm, or about 1 mm. However, embodiments of the present disclosure are not limited thereto.

In certain embodiments, the second length of the second region of the sensor tail 400 can be in a range of about 1 mm to about 3 mm, for example, about 1 mm to about 2.9 mm, about 1 mm to about 2.8 mm, about 1 mm to about 2.7 mm, about 1 mm to about 2.6 mm, about 1 mm to about 2.5 mm, about 1 mm to about 2.5 mm, about 1 mm to about 2.4 mm, about 1 mm to about 2.3 mm, about 1 mm to about 2.2 mm, about 1 mm to about 2.1 mm, or about 1 mm to about 2 mm. In certain embodiments, the second length of the second region can be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, or about 3 mm. However, embodiments of the present disclosure are not limited thereto.

In certain embodiments, the first polymer membrane 410 (e.g., of FIG. 10) can be composed of a first polymer selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and combinations thereof. In certain embodiments, the first polymer membrane 410 can be composed of a first polymer including a first copolymer selected from the group consisting of a polyvinylpyridine-based copolymer, a polyvinylimidazole-based copolymer, and combinations thereof. In certain embodiments, the polyvinylpyridine-based copolymer can be a polyvinylpyridine-co-polystyrene polymer. In certain embodiments, the first polymer membrane 410 can be composed of a polyvinylpyridine-co-polystyrene polymer, wherein the polyvinylpyridine-co-polystyrene polymer includes charged pyridine moieties. In certain embodiments, the first polymer membrane 410 can be composed a polyvinylpyridine-co-styrene copolymer in which a portion of the pyridine nitrogen atoms are functionalized with a non-crosslinked polyethylene glycol tail and a portion of the pyridine nitrogen atoms are functionalized with an alkylsulfonic acid group, e.g., a propylsulfonic acid. In certain embodiments, a derivatized polyvinylpyridine-co-styrene copolymer for utilization in the first polymer membrane 410 can be the 10Q5 polymer as described in U.S. Pat. No. 8,761,857, the content of which is incorporated by reference herein in its entirety.

In certain embodiments, the first polymer membrane 410 (e.g., of FIG. 10) can have a thickness (e.g., dry thickness) in a range of about 0.1 μm to about 1,000 μm, e.g., from about 1 μm to and about 500 μm, about 10 μm to about 300 μm, or about 10 μm to about 100 μm.

In certain embodiments, the second polymer membrane 412 (e.g., of FIG. 10) can have the composition of a therapeutic agent-containing polymer composition described herein. In certain embodiments, the second polymer membrane 412 can include a second polymer including a second polymer selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and combinations thereof. In certain embodiments, the second polymer membrane 412 can include a second copolymer selected from the group consisting of a polyvinylpyridine-based copolymer, a polyvinylimidazole-based copolymer, and a combination thereof. In certain embodiments, the polyvinylpyridine-based copolymer can be a copolymer of vinylpyridine and styrene, or a derivative thereof. In certain embodiments, the second polymer membrane 412 can include a polyvinylpyridine-co-polystyrene polymer.

In certain embodiments, the second polymer membrane 412 (e.g., of FIG. 10) can include poly(4-vinylpyridine-co-styrene). In certain embodiments, the poly(4-vinylpyridine-co-styrene) can include 1-50 mer % of styrene units. In certain embodiments, the poly(4-vinylpyridine-co-styrene) can include 1-30 mer % of styrene units.

In certain embodiments, the second polymer membrane 412 (e.g., of FIG. 10) including a therapeutic agent can have a thickness (e.g., dry thickness) in a range of about 0.1 μm to about 1,000 μm, e.g., from about 1 μm to and about 500 μm, about 10 μm to about 500 μm, about 10 μm to about 400 μm, about 10 μm to about 300 μm, about 10 μm to about 200 μm, or about 10 μm to about 100 μm. In certain embodiments, the second polymer membrane 412 including a therapeutic agent can have a thickness in a range of about 1 μm to about 500 μm. In certain embodiments, the second polymer membrane 412 including a therapeutic agent can have a thickness in a range of about 1 μm to about 400 μm. In certain embodiments, the second polymer membrane 412 including a therapeutic agent can have a thickness in a range of about 1 μm to about 300 μm. In certain embodiments, the second polymer membrane 412 including a therapeutic agent can have a thickness in a range of about 1 μm to about 200 μm. In certain embodiments, the second polymer membrane 412 including a therapeutic agent can have a thickness in a range of about 10 μm to about 200 μm. In certain embodiments, the second polymer membrane 412 including a therapeutic agent can have a thickness in a range of about 10 μm to about 300 μm. In certain embodiments, the second polymer membrane 412 including a therapeutic agent can have a thickness in a range of about 50 μm to about 300 μm.

FIG. 11 illustrates an exemplary sensor tail including a mass-limiting membrane and a drug-eluting membrane according to one or more embodiments of the present disclosure. As shown in FIG. 11, an analyte sensor can include a sensor tail 500 (i.e., in vivo portion) including:
(i) a substrate 502;
(ii) a working electrode 504;
(iii) a counter/reference electrode 506 on the substrate 502;
(iv) a plurality of sensing spots 508a-508f on a surface of the working electrode 504 including a first sensing spot 508a proximal to a tip of the sensor tail among the plurality of sensing spots 508a-508f;
(v) a first region between the tip of the sensor tail 500 and a edge of the first sensing spot 508a with a first length and not including any sensor spot;
(vi) a second region extending from the edge of the first sensing spot 508a along a distal direction away from the tip of the sensor tail and including the plurality of sensing spots 508a-508f with a second length;
(vii) a first polymer membrane 510 coated on the sensor tail 500 in the first region and the second region including the plurality of sensing spots 508a-508f and covering a second polymer membrane 512; and
(viii) the second polymer membrane 512, differing from the first polymer membrane 510, underneath the first polymer membrane 510 in the first region and not covering the second region,
wherein the second polymer membrane 512 including a therapeutic agent.

In certain embodiments, the first length of the first region of the sensor tail 500 can be in a range of about 0.1 mm to about 1 mm, for example, about 0.2 mm to about 1 mm, about 0.3 mm to about 1 mm, about 0.4 mm to about 1 mm, about 0.5 mm to about 1 mm, about 0.6 mm to about 1 mm, or about 0.7 mm to about 1 mm. In certain embodiments, the first length of the first region can be about 0.1 mm, about 0.2 mm, about 0.3 mm, about 0.4 mm, about 0.5 mm, about 0.6 mm, about 0.7 mm, about 0.8 mm, about 0.9 mm, or about 1 mm. However, embodiments of the present disclosure are not limited thereto.

In certain embodiments, the second length of the second region of the sensor tail 500 can be in a range of about 1 mm to about 3 mm, for example, about 1 mm to about 2.9 mm, about 1 mm to about 2.8 mm, about 1 mm to about 2.7 mm, about 1 mm to about 2.6 mm, about 1 mm to about 2.5 mm, about 1 mm to about 2.5 mm, about 1 mm to about 2.4 mm, about 1 mm to about 2.3 mm, about 1 mm to about 2.2 mm, about 1 mm to about 2.1 mm, or about 1 mm to about 2 mm. In certain embodiments, the second length of the second region can be about 1 mm, about 1.1 mm, about 1.2 mm, about 1.3 mm, about 1.4 mm, about 1.5 mm, about 1.6 mm, about 1.7 mm, about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, or about 3 mm. However, embodiments of the present disclosure are not limited thereto.

In certain embodiments, the first polymer membrane 510 can be composed of a first polymer selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and combinations thereof. In certain embodiments, the first polymer membrane 510 can be composed of a first polymer including a first copolymer selected from the group consisting of a polyvinylpyridine-based copolymer, a polyvinylimidazole-based copolymer, and combinations thereof.

In certain embodiments, the polyvinylpyridine-based copolymer can be a polyvinylpyridine-co-polystyrene polymer. In certain embodiments, the first polymer membrane 510 can be composed of a polyvinylpyridine-co-polystyrene polymer, wherein the polyvinylpyridine-co-polystyrene polymer includes charged pyridine moieties. In certain embodiments, the first polymer membrane 510 can be composed a polyvinylpyridine-co-styrene copolymer in which a portion of the pyridine nitrogen atoms are functionalized with a non-crosslinked polyethylene glycol tail and a portion of the pyridine nitrogen atoms are functionalized with an alkylsulfonic acid group, e.g., a propylsulfonic acid. In certain embodiments, a derivatized polyvinylpyridine-co-styrene copolymer for utilization in the first polymer membrane 510 can be the 10Q5 polymer as described in U.S. Pat. No. 8,761,857, the content of which is incorporated by reference herein in its entirety.

In certain embodiments, the first polymer membrane 510 can have a thickness (e.g., dry thickness) in a range of about 0.1 μm to about 1,000 μm, e.g., from about 1 μm to and about 500 μm, about 10 μm to about 300 μm, or about 10 μm to about 100 μm.

In certain embodiments, the second polymer membrane 512 can have the composition of a therapeutic agent-containing polymer composition described herein. In certain embodiments, the second polymer membrane 512 can include a second polymer including a second polymer selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and combinations thereof. In certain embodiments, include a second polymer including a second copolymer selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and combinations thereof. In certain embodiments, the second polymer membrane 512 can include a second copolymer selected from the group consisting of a polyvinylpyridine-based copolymer, a polyvinylimidazole-based copolymer, and a combination thereof. In certain embodiments, the polyvinylpyridine-based copolymer can be a copolymer of vinylpyridine and styrene, or a derivative thereof.

In certain embodiments, the second polymer membrane 512 can include a polyvinylpyridine-co-polystyrene polymer. In certain embodiments, the second polymer membrane 512 can include poly(4-vinylpyridine-co-styrene).

In certain embodiments, the poly(4-vinylpyridine-co-styrene) can include 1-50 mer % of styrene units. In certain embodiments, the poly(4-vinylpyridine-co-styrene) can include 1-30 mer % of styrene units.

In certain embodiments, the second polymer membrane 512 including a therapeutic agent can have a thickness (e.g., dry thickness) in a range of about 0.1 μm to about 1,000 μm, e.g., from about 1 μm to and about 500 μm, about 10 μm to about 500 μm, about 10 μm to about 400 μm, about 10 μm to about 300 μm, about 10 μm to about 200 μm, or about 10 μm to about 100 μm. In certain embodiments, the second polymer membrane 512 including a therapeutic agent can have a thickness in a range of about 1 μm to about 500 μm. In certain embodiments, the second polymer membrane 512 including a therapeutic agent can have a thickness in a range of about 1 μm to about 400 μm. In certain embodiments, the second polymer membrane 512 including a therapeutic agent can have a thickness in a range of about 1 μm to about 300 μm. In certain embodiments, the second polymer membrane 512 including a therapeutic agent can have a thickness in a range of about 1 μm to about 200 μm. In certain embodiments, the second polymer membrane 512 including a therapeutic agent can have a thickness in a range of about 10 m to about 200 μm. In certain embodiments, the second polymer membrane 512 including a therapeutic agent can have a thickness in a range of about 10 μm to about 300 μm. In certain embodiments, the second polymer membrane 512 including a therapeutic agent can have a thickness in a range of about 50 μm to about 300 μm.

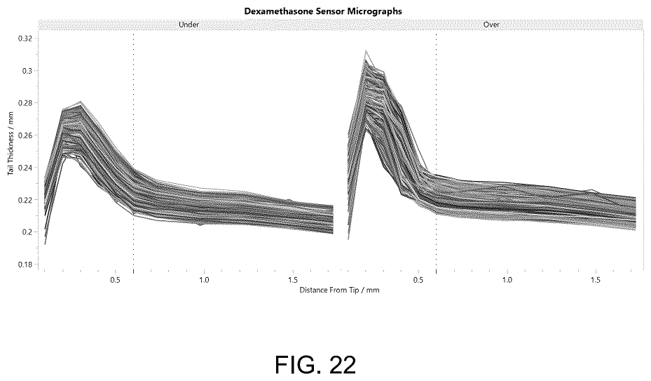

In certain embodiments, the second polymer membrane can have a varied thickness (e.g., over the length of the of the second polymer membrane. As shown in FIG. 22, the more distal region of the second polymer membrane is thicker (e.g., about 0.3 mm from the tip of the sensor tail) than the more proximal region (e.g., about 1.5 mm from the tip of the sensor tail) of the second polymer membrane. In certain embodiments, the ratio of the thinnest point to the thickest point of the second polymer membrane is no greater than about 0.9. In certain embodiments, the ratio of the thinnest point to the thickest point of the second polymer membrane is no greater than about 0.85. In certain embodiments, the ratio of the thinnest point to the thickest point of the second polymer membrane is no greater than about 0.8. In certain embodiments, the ratio of the thinnest point to the thickest point of the second polymer membrane is no greater than about 0.75. In certain embodiments, the ratio of the thinnest point to the thickest point of the second polymer membrane is no greater than about 0.7. In certain embodiments, the ratio of the thinnest point to the thickest point of the second polymer membrane is no greater than about 0.65. In certain embodiments, the ratio of the thinnest point to the thickest point of the second polymer membrane is no greater than about 0.6. In certain embodiments, the ratio of the thinnest point to the thickest point of the second polymer membrane is no greater than about 0.55. In certain embodiments, the ratio of the thinnest point to the thickest point of the second polymer membrane is no greater than about 0.5.

In certain embodiments, the ratio of the thinnest point to the thickest point of the second polymer membrane is less than about 0.9, as shown in FIG. 22. In certain embodiments, the ratio of the thinnest point to the thickest point of the second polymer membrane is less than about 0.85. In certain embodiments, the ratio of the thinnest point to the thickest point of the second polymer membrane less than about 0.8. In certain embodiments, the ratio of the thinnest point to the thickest point of the second polymer membrane less than about 0.75. In certain embodiments, the ratio of the thinnest point to the thickest point of the second polymer membrane less than about 0.7. In certain embodiments, the ratio of the thinnest point to the thickest point of the second polymer membrane less than about 0.65. In certain embodiments, the ratio of the thinnest point to the thickest point of the second polymer membrane less than about 0.6. In certain embodiments, the ratio of the thinnest point to the thickest point of the second polymer membrane less than about 0.55. In certain embodiments, the ratio of the thinnest point to the thickest point of the second polymer membrane less than about 0.5.

In certain embodiments, the first polymer membrane 410 or 510 can be a mass transport limiting membrane. In certain embodiments, the mass transport limiting membrane can be a glucose-limiting membrane.

In certain embodiments, the second polymer membrane 412 or 512 can be a drug-eluting membrane including a therapeutic agent. As disclosed herein, the terms “drug-eluting membrane” and “therapeutic agent-containing polymer composition” are used interchangeably. Further details of therapeutic agent-containing polymer compositions that can be second polymer membranes are described herein in Section IV.

The present disclosure further provides the use of a drug-loading structure to incorporate a therapeutic agent disclosed herein into an analyte sensor. In certain embodiments, due to the limited space on the subcutaneous part of the in vivo analyte sensor, applying/adding a therapeutic agent, such as dexamethasone, which can minimize immune response and therefore improve sensors performance, in the correct region is desired.

In certain embodiments, the present disclosure provides an analyte sensor including a sensor tail (i.e., an in vivo portion) including a drug-loading structure filled with a therapeutic agent-containing polymer composition. In certain embodiments, the drug-loaded structure filled with a therapeutic agent-containing polymer composition is located in a non-sensing region of the sensor tail, which allows the therapeutic agent such as dexamethasone to be released over time but does not affect the sensing chemistry of the active area (e.g., sensing spots) or sensor thickness around the active area (e.g., sensing spots (also referred to active chemistry spots)). For example, but not by way of limitation, one or more aspects of embodiments of the present disclosure are directed toward an analyte sensor, where the analyte sensor includes a sensor tail (i.e., in vivo portion) including a non-sensing region and an active sensing region (e.g., comprising a plurality of sensing spots), wherein the non-sensing region surrounds the active sensing region and can include a drug-loading structure filled with a therapeutic agent-containing polymer composition.

In certain embodiments, an analyte sensor of the present disclosure includes one or more drug-loading structures. For example, but not by way of limitation, an analyte sensor of the present disclosure can include (i) a sensor tail (i.e., an in vivo portion) including at least a first working electrode on a substrate; (ii) an active area disposed upon a surface of the first working electrode for detecting an analyte; (iii) a mass transport limiting membrane permeable to the analyte that coats at least the active area; (iv) a counter/reference electrode on the substrate; and (v) one or more drug-loading structures.

FIG. 3 illustrates a schematic view of an exemplary sensor tail (i.e., an in vivo portion) according to one or more embodiments of the present disclosure. As shown in FIG. 3, an analyte sensor can include a sensor tail 300 including a non-sensing region 302 and an active sensing region 304 comprising a sensing layer (e.g., including a plurality of sensing spots 306), wherein the non-sensing region 302 surrounds the active sensing region 304 and can include a drug-loading structure, such as 308a shown in of FIG. 3B, 308b shown in FIG. 3C, or 308c shown in FIG. 3D, filled with a therapeutic agent-containing polymer composition 310. In certain embodiments, the one or more drug-loading structures can be located on the substrate of the sensor tail.

In certain embodiments, the one or more drug-loading structures can be located on the substrate of the sensor tail, e.g., as shown in FIGS. 3B-3D.

In certain embodiments, the drug-loading structure can include at least one selected from a slot, a hole, a pore, a groove, a depression or a combination thereof.

In certain embodiments, the drug-loading structure can include at least one slot.

In certain embodiments, the drug-loading structure can include at least one hole.

In certain embodiments, the drug-loading structure can include at least one pore.

In certain embodiments, the drug-loading structure can include at least one groove.

In certain embodiments, the drug-loading structure can include at least one depression.

In certain embodiments, the drug-loading structure can include two slots symmetrically arranged along the plurality of sensing spots, e.g., as shown in FIG. 3B. In certain embodiments, the drug-loading structure can include a plurality of holes arranged (e.g., symmetrically arranged) around the plurality of sensing spots, e.g., as shown in FIG. 3C. For example, but not by way of limitation, the plurality of holes can include 1 or more holes, 2 or more holes, 3 or more holes, 4 or more holes, 5 or more holes, 6 or more holes, 7 or more holes, 8 or more holes, 9 or more holes, 10 or more holes, 11 or more holes, 12 or more holes, 13 or more holes, 14 or more holes, 15 or more holes, 16 or more holes, 17 or more holes, 18 or more holes, 19 or more holes, 20 or more holes, 21 or more holes, 22 or more holes, 23 or more holes, 24 or more holes, 25 or more holes, 26 or more holes, 27 or more holes, 28 or more holes, 29 or more holes or 30 or more holes. In certain embodiments, the hole is in a shape of a circle, a shape of a regular polygon, or a shape of an irregular polygon. In certain embodiments, the hole is in a shape of a circle, a shape of a regular polygon, or a shape of a rectangle, e.g., as shown in FIG. 3D. In certain embodiments, the hole is a through hole.

In certain embodiments, the slot is in a shape of an oval, a shape of a polygon, or an irregular shape. In certain embodiments, the slot is a through slot.

In certain embodiments, a depth of the groove or the depression can be about one fourth to about three fourths of a thickness of the sensor tail. In certain embodiments, the surface of the distal tip of the sensor tail is ablated (e.g., laser ablated) and/or is planed to create a depression or groove. In certain embodiments, the surface of the distal tip of the sensor tail is ablated and/or is planed up to the sensing layer (e.g., the one or more sensing spots) to create a depression or groove. For example, but not by way of limitation, the region between the tip of the sensor tail and the beginning of the sensing layer (referred to as d3 in FIG. 7) is ablated and/or is planed to create a depression or groove to generate a drug-loading structure that can be filled and/or covered with a therapeutic agent-containing polymer composition.

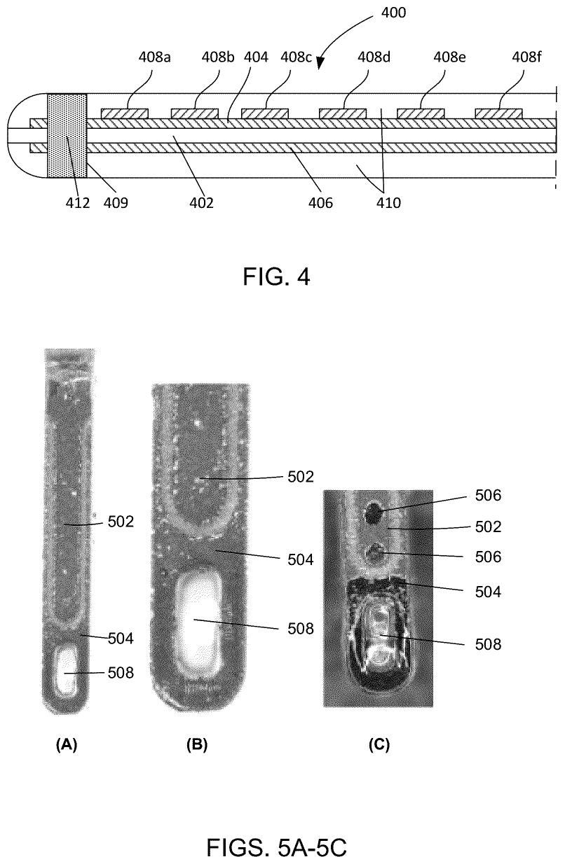

FIG. 4 illustrates a schematic view of an exemplary analyte sensor according to one or more embodiments of the present disclosure. As shown in FIG. 4, an analyte sensor can include a sensor tail 400 (i.e., in vivo portion) including:
(i) a substrate 402;
(ii) a working electrode 404;
(iii) a counter/reference electrode 406 on the substrate 402;
(iv) a plurality of sensing spots 408a-408f on a surface of the working electrode 404;
(v) a polymer membrane 410 covering the plurality of sensing spots 408a-408f;
(vi) a drug-loading structure 409 filled with a therapeutic agent-containing polymer composition 412 (e.g., a therapeutic agent-containing polymer composition).

In certain embodiments, the polymer membrane 410 can be a mass-limiting membrane. In certain embodiments, mass-limiting membrane 410 can refer to the mass-limiting membrane disclosed elsewhere herein (e.g., in Section III), which will not be repeated herein for conciseness. In certain embodiments, the mass-limiting membrane can be a glucose-limiting membrane. In certain embodiments, the polymer membrane 410 can have a thickness (e.g., dry thickness) in a range of about 0.1 μm to about 1,000 μm, e.g., from about 1 μm to and about 500 μm, about 10 μm to about 300 μm, about 10 μm to about 100 μm or about 10 μm to about 500 μm.

In certain embodiments, the drug-loading structure 409 can be an indent, an impression, a hole or a slot. In certain embodiments, therapeutic agent-containing polymer composition 412 fills the drug-loading structure 409. In certain embodiments, the therapeutic agent-containing polymer composition 412 can continuously release a therapeutic agent, such as dexamethasone, a derivative thereof, or a salt thereof, at a predetermined drug delivery rate for a predetermined days such as for at least 30 days. The detailed description of the therapeutic agent-containing polymer composition 412 can refer to the therapeutic agent-containing polymer composition disclosed elsewhere in the present disclosure (e.g., in Section IV).

FIG. 5 illustrates images of an exemplary analyte sensor according to one or more embodiments of the present disclosure. FIG. 5A illustrates that an analyte sensor can include a sensor tail including an active sensing region 502 and a non-sensing region 504 including a drug-loading structure 508 filled with a therapeutic-agent-containing polymer composition. FIG. 5B is a magnified version of FIG. 5A and shows the detailed structures of the sensor tail. FIG. 5C illustrates that an analyte sensor can include a sensor tail including an active sensing region 502 (e.g., including a plurality of sensing spots 506) and a non-sensing region 504 including a drug-loading structure 508 filled with a therapeutic-agent-containing polymer composition. The detailed description of the drug-loading structure 508 filled (or loaded) with the therapeutic agent-containing polymer composition can refer to the drug-loading structure and the therapeutic agent-containing polymer composition disclosed herein.

In certain embodiments, the drug-loading structure can have a total area of about 0.01 mm² to about 3.0 mm², e.g., about 0.01 mm² to about 2.0 mm², 0.01 mm² to about 1.0 mm², about 0.01 mm² to about 0.5 mm², about 0.01 mm² to about 0.3 mm², or about 0.01 mm² to about 0.1 mm², or any values in between. In certain embodiments, the total area of the drug-loading structure can refer to the sum of an area of all sub-loading structures such as slots, holes, pores, grooves, and/or depressions. The area of the drug-loading structure is an area of the drug-loading structure in a plan view, for example, the sum of an area of all sub-loading structures in a plan view.

As described herein, examples of therapeutic agent-containing polymer compositions (also referred to as drug-eluting membranes and second polymer membranes) that can be incorporated into an analyte sensor of the present disclosure are described in Section IV. In certain embodiments, the therapeutic agent-containing polymer composition incorporated into an analyte sensor can include an amount of the therapeutic agent, e.g., dexamethasone or a derivative thereof, in a range of 0.01 wt %-50 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 0.01 wt %-40 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 1 wt %-40 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 5 wt %-40 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 10 wt %-40 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 20 wt %-40 wt % based on the total weight of the therapeutic agent-containing polymer composition. In certain embodiments, the therapeutic agent-containing polymer composition incorporated into an analyte sensor can include an amount of the therapeutic agent, e.g., dexamethasone or a derivative thereof, in a range of 30 wt %-40 wt % based on the total weight of the therapeutic agent-containing polymer composition.

In certain embodiments, the therapeutic agent-containing polymer composition incorporated into an analyte sensor can include an amount of the therapeutic agent, e.g., dexamethasone or a derivative thereof, in a range of 1 wt %-50 wt % based on the total weight of the therapeutic agent-containing polymer composition.

In certain embodiments, the therapeutic agent-containing polymer composition incorporated into an analyte sensor can include from about 0.0005 mg to about 0.2 mg of the therapeutic agent, e.g., dexamethasone, or any values in between. In certain embodiments, the therapeutic agent-containing polymer composition can include about 0.0005 mg, about 0.001 mg, about 0.005 mg, about 0.01 mg, about 0.05 mg, about 0.1 mg or about 0.2 mg of the therapeutic agent, e.g., dexamethasone. In certain embodiments, the therapeutic agent-containing polymer composition incorporated into an analyte sensor can include from about 0.1 μg to about 20 μg of the therapeutic agent. In certain embodiments, the therapeutic agent-containing polymer composition incorporated into an analyte sensor can include from about 0.1 μg to about 200 μg of the therapeutic agent, e.g., from about 0.5 μg to about 200 μg, from about 1 μg to about 200 μg, from about 1.5 μg to about 200 μg, from about 2.0 μg to about 200 μg, from about 2.5 μg to about 200 μg, from about 3 μg to about 200 μg, from about 4 μg to about 200 μg, from about 5 μg to about 200 μg, from about 10 μg to about 200 μg, from about 15 μg to about 200 μg, from about 20 μg to about 200 μg, from about 25 μg to about 200 μg, from about 30 μg to about 200 μg, from about 35 μg to about 200 μg, from about 40 μg to about 200 μg, from about 45 μg to about 200 μg, from about 50 μg to about 200 μg, from about 55 μg to about 200 μg, from about 60 μg to about 200 μg, from about 65 μg to about 200 μg, from about 70 μg to about 200 μg, from about 75 μg to about 200 μg, from about 80 μg to about 200 μg, from about 85 μg to about 200 μg, from about 90 μg to about 200 μg, from about 95 μg to about 200 μg, from about 100 μg to about 200 μg, from about 110 μg to about 200 μg, from about 120 μg to about 200 μg, from about 130 μg to about 200 μg, from about 140 μg to about 200 μg, from about 150 μg to about 200 μg, from about 160 μg to about 200 μg, from about 170 μg to about 200 μg, from about 180 μg to about 200 μg, from about 190 μg to about 200 μg, from about 0.1 μg to about 190 μg, from about 0.1 μg to about 180 μg, from about 0.1 μg to about 170 μg, from about 0.1 μg to about 160 μg, from about 0.1 μg to about 150 μg, from about 0.1 μg to about 140 μg, from about 0.1 μg to about 130 μg, from about 0.1 μg to about 120 μg, from about 0.1 μg to about 110 μg, from about 0.1 μg to about 100 μg, from about 1 μg to about 150 μg, from about 5 μg to about 150 μg or from about 5 μg to about 120 μg. In certain embodiments, the therapeutic agent-containing polymer composition incorporated into an analyte sensor can include from about 1 μg to about 100 μg of the therapeutic agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In certain embodiments, the therapeutic agent-containing polymer composition incorporated into an analyte sensor can include from about 1 μg to about 5 μg of the therapeutic agent. In certain embodiments, the therapeutic agent-containing polymer composition incorporated into an analyte sensor can include from about 1 μg to about 10 μg of the therapeutic agent. In certain embodiments, the therapeutic agent-containing polymer composition incorporated into an analyte sensor can include from about 1 μg to about 15 μg of the therapeutic agent. In certain embodiments, the therapeutic agent-containing polymer composition incorporated into an analyte sensor can include from about 1 μg to about 20 μg of the therapeutic agent. In certain embodiments, the therapeutic agent-containing polymer composition incorporated into an analyte sensor can include from about 5 μg to about 20 μg of the therapeutic agent. In certain embodiments, the therapeutic agent-containing polymer composition incorporated into an analyte sensor can include from about 1 μg to about 30 μg of the therapeutic agent. In certain embodiments, the therapeutic agent-containing polymer composition incorporated into an analyte sensor can include from about 5 μg to about 10 μg of the therapeutic agent. In certain embodiments, the therapeutic agent-containing polymer composition incorporated into an analyte sensor can include from about 5 μg to about 15 μg of the therapeutic agent. In certain embodiments, the therapeutic agent-containing polymer composition incorporated into an analyte sensor can include from about 5 μg to about 20 μg of the therapeutic agent. In certain embodiments, the therapeutic agent-containing polymer composition incorporated into an analyte sensor can include from about 5 μg to about 25 μg of the therapeutic agent. In certain embodiments, the therapeutic agent-containing polymer composition incorporated into an analyte sensor can include from about 5 μg to about 30 μg of the therapeutic agent. In certain embodiments, the therapeutic agent-containing polymer composition incorporated into an analyte sensor can include from about 0.1 μg to about 30 μg of the therapeutic agent. In certain embodiments, the therapeutic agent-containing polymer composition incorporated into an analyte sensor can include from about 0.1 μg to about 20 μg of the therapeutic agent. In certain embodiments, the therapeutic agent-containing polymer composition incorporated into an analyte sensor can include from about 0.1 μg to about 15 μg of the therapeutic agent. In certain embodiments, the therapeutic agent-containing polymer composition incorporated into an analyte sensor can include from about 0.1 μg to about 10 μg of the therapeutic agent. In certain embodiments, the therapeutic agent-containing polymer composition incorporated into an analyte sensor can include from about 0.1 μg to about 5 μg of the therapeutic agent.

In certain embodiments, the therapeutic agent-containing polymer composition incorporated into an analyte sensor can include from about 0.01 μg to about 5 μg of the therapeutic agent.

In certain embodiments, the therapeutic agent-containing polymer composition can include less than about 10 μg of the therapeutic agent.

In certain embodiments, the therapeutic agent-containing polymer composition can include less than about 5 μg of the therapeutic agent.

In certain embodiments, the therapeutic agent-containing polymer composition incorporated into an analyte sensor can continuously release the therapeutic agent at a drug delivery rate of about 0.001 μg/day to about 1 mg/day of the therapeutic agent, e.g., dexamethasone, or any values in between.

In certain embodiments, the therapeutic agent-containing polymer composition incorporated into an analyte sensor can continuously release the therapeutic agent at a drug delivery rate (e.g., an average drug delivery rate) of about 0.001 μg/day to about 5 μg/day of the therapeutic agent, e.g., dexamethasone, or any values in between.

In certain embodiments, the therapeutic agent-containing polymer composition incorporated into an analyte sensor can continuously release the therapeutic agent at a drug delivery rate (e.g., an average drug delivery rate) of about 0.001 μg/day to about 1 μg/day of the therapeutic agent, e.g., dexamethasone, or any values in between.

In certain embodiments, the therapeutic agent-containing polymer composition incorporated into an analyte sensor can continuously release the therapeutic agent at a drug delivery rate (e.g., an average drug delivery rate) of about 0.001 μg/day to about 0.1 μg/day of the therapeutic agent, e.g., dexamethasone, or any values in between.

In certain embodiments, the therapeutic agent-containing polymer composition incorporated into an analyte sensor can continuously release the therapeutic agent at a drug delivery rate (e.g., an average drug delivery rate) of about 0.01 μg/day to about 100 μg/day of the therapeutic agent, e.g., dexamethasone, or any values in between.

In certain embodiments, the therapeutic agent-containing polymer composition incorporated into an analyte sensor can continuously release the therapeutic agent at a drug delivery rate (e.g., an average drug delivery rate) of about 0.01 μg/day to about 10 μg/day of the therapeutic agent, e.g., dexamethasone, or any values in between.

In certain embodiments, the therapeutic agent-containing polymer composition incorporated into an analyte sensor can continuously release the therapeutic agent at a drug delivery rate of about 0.01 μg/day to about 1 mg/day of the therapeutic agent, e.g., dexamethasone, or any values in between.

In certain embodiments, the therapeutic agent-containing polymer composition incorporated into an analyte sensor can continuously release the therapeutic agent at a drug delivery rate of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the therapeutic agent, e.g., dexamethasone, or any values in between. In certain embodiments, the therapeutic agent-containing polymer composition incorporated into an analyte sensor can continuously release the therapeutic agent at a drug delivery rate of about 0.2 μg/day to about 5 μg/day of the therapeutic agent, e.g., dexamethasone. In certain embodiments, the therapeutic agent-containing polymer composition incorporated into an analyte sensor can continuously release the therapeutic agent at a drug delivery rate of about 0.2 μg/day to about 2 μg/day of the therapeutic agent, e.g., dexamethasone. In certain embodiments, the therapeutic agent-containing polymer composition incorporated into an analyte sensor can continuously release the therapeutic agent at a drug delivery rate of about 0.2 μg/day to about 1 μg/day of the therapeutic agent, e.g., dexamethasone. In certain embodiments, the therapeutic agent-containing polymer composition incorporated into an analyte sensor can continuously release the therapeutic agent at a predetermined drug delivery rate to achieve desirable therapeutic results such as reducing, minimizing, preventing, and/or inhibiting the inflammation and/or infection.

In certain embodiments, the therapeutic agent-containing polymer composition incorporated into an analyte sensor can continuously release the therapeutic agent at a predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days.

In certain embodiments, the therapeutic agent-containing polymer composition incorporated into an analyte sensor can continuously release the therapeutic agent at a predetermined drug delivery rate for a predetermined days such as for at least 30 days.

In certain embodiments, a therapeutic agent-containing polymer composition of the present disclosure when incorporated into an analyte sensor releases about 40% to about 80% of the therapeutic agent present within the composition (e.g., the total amount of therapeutic agent loaded into the composition), e.g., within a period of about 30-31 days. For example, but not by way by way of limitation, a composition of the present disclosure when incorporated into an analyte sensor releases about 45% to about 80% of the therapeutic agent, releases about 50% to about 80% of the therapeutic agent, releases about 55% to about 80% of the therapeutic agent, releases about 60% to about 80% of the therapeutic agent, releases about 65% to about 80% of the therapeutic agent, releases about 70% to about 80% of the therapeutic agent, releases about 75% to about 80% of the therapeutic agent, releases about 40% to about 75% of the therapeutic agent, releases about 40% to about 70% of the therapeutic agent, releases about 40% to about 65% of the therapeutic agent, releases about 40% to about 60% of the therapeutic agent, releases about 40% to about 55% of the therapeutic agent, releases about 40% to about 50% of the therapeutic agent, releases about 40% to about 45% of the therapeutic agent, releases about 45% to about 75% of the therapeutic agent, releases about 50% to about 75% of the therapeutic agent, releases about 55% to about 80% of the therapeutic agent or releases about 60% to about 75% of the therapeutic agent present within the composition (e.g., the total amount of therapeutic agent loaded into the composition), e.g., within a period of about 30-31 days.

In certain embodiments, no more than about 90% of therapeutic agent present within a composition (e.g., the total amount of therapeutic agent loaded into the composition) incorporated into an analyte sensor is released, e.g., within a period of about 30-31 days. In certain embodiments, no more than about 95% of the therapeutic agent present within the composition (e.g., the total amount of therapeutic agent loaded into the composition) incorporated into an analyte sensor is released, e.g., within a period of about 30-31 days. In certain embodiments, no more than about 80% of the therapeutic agent present within the composition (e.g., the total amount of therapeutic agent loaded into the composition) incorporated into an analyte sensor is released, e.g., within a period of about 30-31 days. In certain embodiments, no more than about 75% of the therapeutic agent present within the composition (e.g., the total amount of therapeutic agent loaded into the composition) incorporated into an analyte sensor is released, e.g., within a period of about 30-31 days. In certain embodiments, no more than about 70% of the therapeutic agent present within the composition (e.g., the total amount of therapeutic agent loaded into the composition) incorporated into an analyte sensor is released, e.g., within a period of about 30-31 days. In certain embodiments, no more than about 65% of the therapeutic agent present within the composition (e.g., the total amount of therapeutic agent loaded into the composition) incorporated into an analyte sensor is released, e.g., within a period of about 30-31 days. In certain embodiments, no more than about 60% of the therapeutic agent present within the composition (e.g., the total amount of therapeutic agent loaded into the composition) incorporated into an analyte sensor is released, e.g., within a period of about 30-31 days. In certain embodiments, no more than about 55% of the therapeutic agent present within the composition (e.g., the total amount of therapeutic agent loaded into the composition) incorporated into an analyte sensor is released, e.g., within a period of about 30-31 days. In certain embodiments, no more than about 50% of the therapeutic agent present within the composition (e.g., the total amount of therapeutic agent loaded into the composition) incorporated into an analyte sensor is released, e.g., within a period of about 30-31 days.

In certain embodiments, no more than about 30%, 35%, 40%, 45%, 50%, 55%, 60%, 65%, 70% or 75% of the therapeutic agent present within the composition (e.g., the total amount of therapeutic agent loaded into the composition) incorporated into an analyte sensor is released in the first 5, 6 or 7 days after insertion. In certain embodiments, no more than about 30% of the therapeutic agent present within the composition (e.g., the total amount of therapeutic agent loaded into the composition) incorporated into an analyte sensor is released in the first 5 days after insertion. In certain embodiments, no more than about 30% of the therapeutic agent present within the composition (e.g., the total amount of therapeutic agent loaded into the composition) incorporated into an analyte sensor is released in the first 6 days after insertion. In certain embodiments, no more than about 30% of the therapeutic agent present within the composition (e.g., the total amount of therapeutic agent loaded into the composition) incorporated into an analyte sensor is released in the first 7 days after insertion. In certain embodiments, no more than about 35% of the therapeutic agent present within the composition (e.g., the total amount of therapeutic agent loaded into the composition) incorporated into an analyte sensor is released in the first 5 days after insertion. In certain embodiments, no more than about 35% of the therapeutic agent present within the composition (e.g., the total amount of therapeutic agent loaded into the composition) incorporated into an analyte sensor is released in the first 6 days after insertion. In certain embodiments, no more than about 35% of the therapeutic agent present within the composition (e.g., the total amount of therapeutic agent loaded into the composition) incorporated into an analyte sensor is released in the first 7 days after insertion. In certain embodiments, no more than about 40% of the therapeutic agent present within the composition (e.g., the total amount of therapeutic agent loaded into the composition) incorporated into an analyte sensor is released in the first 5 days after insertion. In certain embodiments, no more than about 40% of the therapeutic agent present within the composition (e.g., the total amount of therapeutic agent loaded into the composition) incorporated into an analyte sensor is released in the first 6 days after insertion. In certain embodiments, no more than about 40% of the therapeutic agent present within the composition (e.g., the total amount of therapeutic agent loaded into the composition) incorporated into an analyte sensor is released in the first 7 days after insertion.

Further descriptions of therapeutic agent-containing polymer compositions (also referred to as drug-eluting membranes and second polymer membranes) that can be incorporated into an analyte sensor are disclosed elsewhere in the present disclosure, e.g., Section IV, which will not be repeated herein for conciseness.

### VI. Methods of Manufacture

The present disclosure further provides methods for preparing an analyte sensor including one or more therapeutic agents. For example, but not by way of limitation, the present disclosure provides methods for preparing an analyte sensor including one or more therapeutic agent-containing compositions of the present disclosure.

In certain embodiments, the present disclosure provides methods for preparing analyte sensors that include one or more therapeutic agents, e.g., present in a therapeutic agent-containing polymer composition, disposed upon or incorporated into a component of the analyte sensor (e.g., an in vivo portion of the analyte sensor). For example, but not by way of limitation, methods of the present disclosure include the dispensing of a therapeutic agent-containing polymer composition upon a surface of an electrode (e.g., a counter/reference electrode (e.g., 216 of FIG. 18A) and/or a working electrode (e.g., 214 of FIG. 18A)), an insulating material (e.g., a dielectric material (e.g., 219a-c of FIG. 18A)), a substrate (e.g., 212 of FIG. 18A), a mass transport limiting membrane (e.g., 220 of FIG. 18A) and a distal tip of the analyte sensor (e.g., 412 of FIG. 10 and 513 of FIG. 11). In certain embodiments, a therapeutic agent-containing polymer composition is not disposed upon the active layer (e.g., the multiple sensing spots). In certain embodiments, a therapeutic agent-containing polymer composition is disposed upon a structure or component of the analyte sensor that is adjacent to the active layer (e.g., the multiple sensing spots).

In certain embodiments, a therapeutic agent-containing polymer composition of the present disclosure disposed upon or incorporated into a structure or component of the analyte sensor (e.g., an in vivo portion of the analyte sensor) by a screen printing, a rotary printing, a jetting, an aerosol deposition, microdispensing, a spray coating, a dip coating, a drop-casting, a spin coating or a brush coating process.

In certain embodiments, depositing a therapeutic agent-containing polymer composition of the present disclosure onto a structure or component of the sensor tail (e.g., an in vivo portion of the analyte sensor) can be performed by dipping.

In certain embodiments, depositing a therapeutic agent-containing polymer composition of the present disclosure onto a structure or component of the sensor tail (e.g., an in vivo portion of the analyte sensor) can be performed by drop-casting.

In certain embodiments, depositing a therapeutic agent-containing polymer composition of the present disclosure onto a structure or component of the sensor tail (e.g., an in vivo portion of the analyte sensor) can be performed by aerosol deposition.

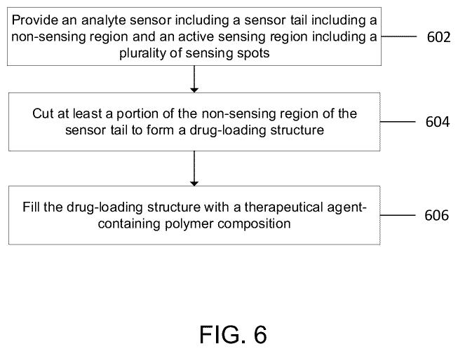

In one aspect, the present disclosure provides methods for preparing an analyte sensor including one or more therapeutic agents that includes the generation of drug-loading structures. FIG. 6 illustrates a flow chart of an exemplary method for preparing an analyte sensor according to one or more embodiments of the present disclosure. As shown in FIG. 6, an exemplary method for preparing analyte sensor can include:
(602) providing an analyte sensor including a sensor tail including a non-sensing region and an active sensing region (e.g., comprising a plurality of sensing spots);
(604) cutting at least a portion of the non-sensing region of the sensor tail to form a drug-loading structure; and
(606) filling (or loading) the drug-loading structure with a therapeutic agent-containing polymer composition.

In certain embodiments, the active sensing region includes the analyte-responsive active area. In certain embodiments, the analyte-responsive active area includes two or more sensing spots.

In certain embodiments, the cutting of at least a portion of the non-sensing region of the sensor tail to form a drug-loading structure can include removing the at least a portion of the non-sensing region of the sensor tail by a laser-cutting, photolithography, ion beam lithography, etching (e.g., dry-etching or wet-etching), ablation, planing etc. to form the drug-loading structure.

In certain embodiments, the cutting of at least a portion of the non-sensing region of the sensor tail to form a drug-loading structure can include removing the at least a portion of the non-sensing region of the sensor tail by a laser-cutting.

In certain embodiments, the cutting of at least a portion of the non-sensing region of the sensor tail to form a drug-loading structure can include removing the at least a portion of the non-sensing region of the sensor tail by ablation.

In certain embodiments, the cutting of at least a portion of the non-sensing region of the sensor tail to form a drug-loading structure can include removing the at least a portion of the non-sensing region of the sensor tail by planing.

In certain embodiments, filling (or loading) the drug-loading structure with a therapeutic agent-containing polymer composition can include filling the drug-loading structure into the drug-loading structure with the therapeutic agent-containing polymer composition by dipping, spraying, aerosol deposition, jetting, drop-casting, etc.

In certain embodiments, the filling (or loading) the drug-loading structure with a therapeutic agent-containing polymer composition can include filling (or loading) the drug-loading structure into the drug-loading structure with the therapeutic agent-containing polymer composition by dipping.

In certain embodiments, the filling (or loading) the drug-loading structure with a therapeutic agent-containing polymer composition can include filling (or loading) the drug-loading structure into the drug-loading structure with the therapeutic agent-containing polymer composition by aerosol deposition.

In certain embodiments, the drug-loading structure can include at least one selected from a slot, a hole, a pore, a groove, an impression, an indent and a depression. In certain embodiments, the drug-loading structure can include at least one slot. In certain embodiments, the drug-loading structure can include at least one hole. In certain embodiments, the drug-loading structure can include at least one pore. In certain embodiments, the drug-loading structure can include at least one groove. In certain embodiments, the drug-loading structure can include at least one depression. Further details regarding drug-loading structures are described in Section V herein.

In certain embodiments, the drug-loading structure can include two slots symmetrically arranged along the plurality of sensing spots.

In certain embodiments, the drug-loading structure can include a plurality of holes symmetrically arranged around the plurality of sensing spots. In certain embodiments, the hole is in a shape of a circle, a shape of a regular polygon, or a shape of an irregular polygon. In certain embodiments, the hole is a through hole.

In certain embodiments, the slot is in a shape of an oval, a shape of a polygon, or an irregular shape. In certain embodiments, the slot is a through slot.

In certain embodiments, a depth of the groove or the depression can be about one fourth to about three fourth of a thickness of the sensor tail.

In certain embodiments, the drug-loading structure can have a total area of about 0.01 mm² to about 3.0 mm², e.g., about 0.01 mm² to about 2.0 mm², 0.01 mm² to about 1.0 mm², about 0.01 mm² to about 0.5 mm², about 0.01 mm² to about 0.3 mm², or about 0.01 mm² to about 0.1 mm², or any values in between. The total area of the drug-loading structure can refer to the sum of an area of all sub-loading structures such as slots, holes, pores, grooves, and/or depressions. The area of the drug-loading structure is an area of the drug-loading structure in a plan view, for example, the sum of an area of all sub-loading structures in a plan view.

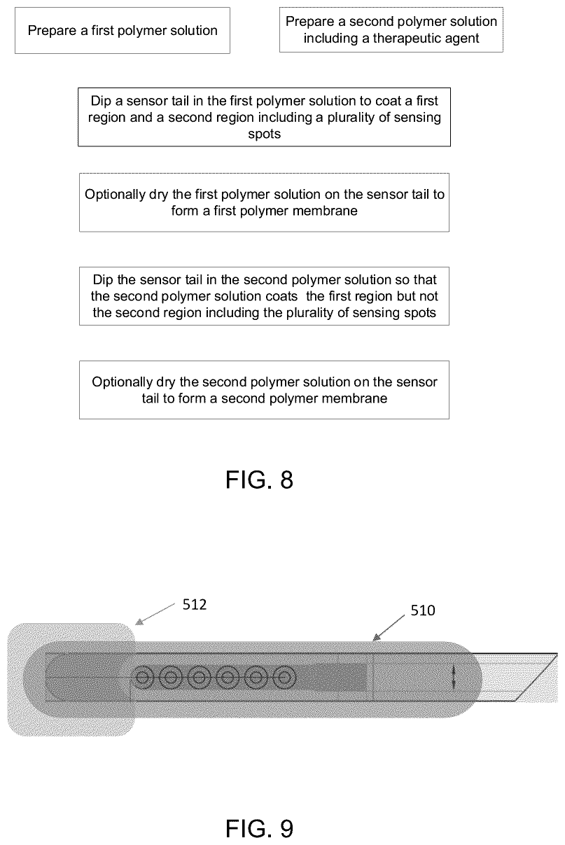

The present disclosure further provides methods for depositing a therapeutic agent at the distal region of a sensor tail (e.g., in vivo portion of an analyte sensor). FIG. 8 illustrates steps, tasks, or acts of a method for coating an analyte sensor according to one or more embodiments of the present disclosure. FIG. 8 illustrates a schematic view of a coated analyte sensor according to one or more embodiments of the present disclosure.

Here, the listing of steps, tasks, or acts in a particular order should not necessarily means that the invention or claims require that particular order. That is, the general rule that unless the steps, tasks, or acts of a method (e.g., a method claim) actually recite an order, the steps, tasks, or acts should not be construed to require one.

In certain embodiments, a method for coating an analyte sensor of the present disclosure includes:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer or providing a first polymer solution comprising a first polymer;
(iii) preparing a second polymer solution comprising a second polymer and a therapeutic agent or providing a second polymer solution comprising a second polymer and a therapeutic agent;
(iv) contacting the in vivo portion with the first polymer solution to coat the first region and the second region to generate a first polymer membrane; and
(v) contacting the first region with the second polymer solution to coat the first region but not the second region to generate a second polymer membrane comprising the therapeutic agent.

In certain embodiments, contacting the in vivo portion with the first polymer solution comprises a screen printing, a rotary printing, a jetting, an aerosol deposition, a spray coating, a dip coating, a drop-casting, a spin coating or a brush coating process.

In certain embodiments, contacting the in vivo portion with the first polymer solution comprises a dip coating process.

In certain embodiments, contacting the first region with the second polymer solution comprises a screen printing, a rotary printing, a jetting, an aerosol deposition, a spray coating, a dip coating, a drop-casting, a spin coating or a brush coating process.

In certain embodiments, contacting the first region with the second polymer solution comprises a dip coating process.

In certain embodiments, contacting the in vivo portion with the first solution is performed before contacting the first region with the second polymer solution. In certain embodiments, contacting the first region with the second polymer solution is performed before contacting the in vivo portion with the first polymer solution.

In certain embodiments, a method for manufacturing an analyte sensor comprising a therapeutic agent includes:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer or providing a first polymer solution comprising a first polymer;
(iii) preparing a second polymer solution comprising a second polymer and a therapeutic agent or providing a second polymer solution comprising a second polymer and a therapeutic agent;
(iv) dipping the in vivo portion in the first polymer solution to coat the first region and the second region to generate a first polymer membrane; and
(v) dipping the first region in the second polymer solution to coat the first region and not the second region to generate a second polymer membrane comprising the therapeutic agent on top of the first polymer membrane.

In certain embodiments, a method for manufacturing an analyte sensor comprising a therapeutic agent includes:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer or providing a first polymer solution comprising a first polymer;
(iii) preparing a second polymer solution comprising a second polymer and a therapeutic agent or providing a second polymer solution comprising a second polymer and a therapeutic agent;
(iv) dipping the first region in the second polymer solution to coat the first region and not the second region to generate a second polymer membrane comprising the therapeutic agent; and
(v) dipping the in vivo portion in the first polymer solution to coat the first region and the second region to generate a first polymer membrane on top of the second polymer membrane.

As shown in FIG. 8, according to one or more embodiments of the present disclosure, a method for coating an analyte sensor including a sensor tail (e.g., an in vivo portion configured to reside below a skin surface of a subject and in contact with interstitial fluid of the subject) including a first region not including any sensing spot and a second region including a plurality of sensing spots, can include:
(i) preparing a first polymer solution and a second polymer solution differing from the first polymer solution and including a therapeutic agent;
(ii) dipping the sensor tail in the first polymer solution to coat the first region and the second region including the plurality of sensing spots;
   optionally drying the first polymer solution on the sensor tail to form a first polymer membrane;
(iii) dipping the sensor tail in the second polymer solution so that the second polymer solution coats the first region of the sensor tail but not the second region including the plurality of sensor spots; and
   optionally drying the second polymer solution on the sensor tail to form a second polymer membrane.

In certain embodiments, methods of the present disclosure can further include repeating the dipping the sensor tail in the first polymer solution and then optionally drying the first polymer solution on the sensor tail to form the first polymer membrane until the first polymer membrane, for example, first polymer membrane (e.g., 510 of FIG. 9 or 410 of FIG. 10), reaches a first set or predetermined thickness (e.g., prior to dipping the sensor tail in the second polymer solution). In certain embodiments, the method can further include repeating the dipping the sensor tail in the second polymer solution and then optionally drying the second polymer solution on the sensor tail to form the second polymer membrane until the second polymer membrane, for example, second polymer membrane (e.g., 512 of FIG. 9 or 412 of FIG. 10), reaches a second set or predetermined thickness (e.g., after formation of the first polymer membrane).

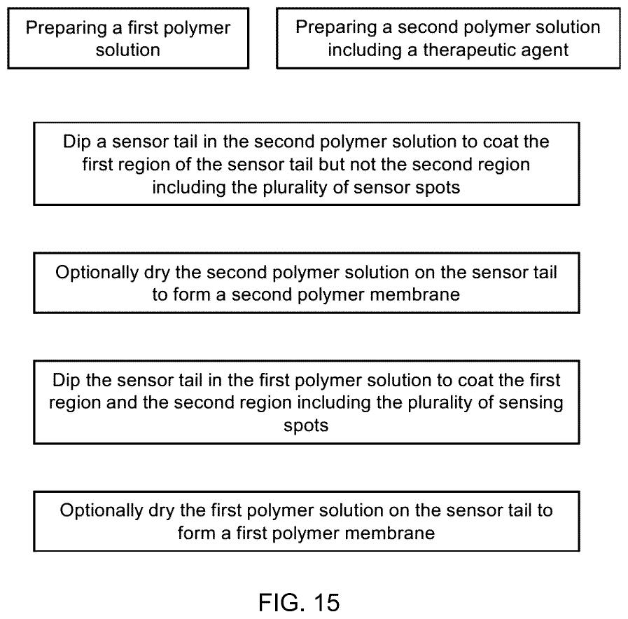

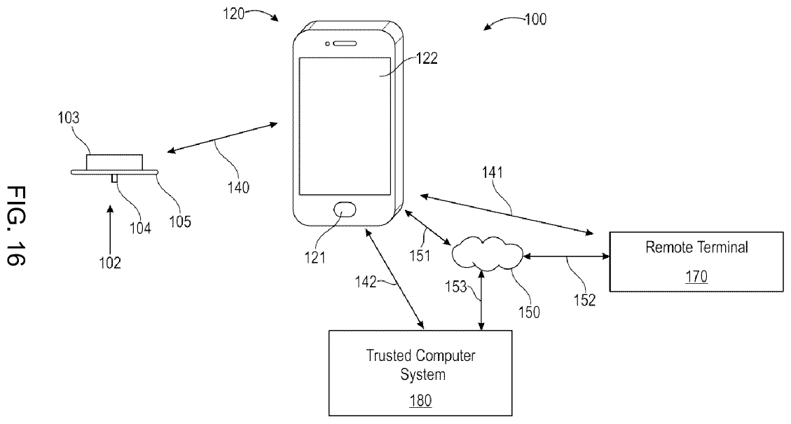

Alternatively, in certain embodiments as shown in FIG. 15, a method for coating an analyte sensor including a sensor tail including a first region not including any sensing spot and a second region including a plurality of sensing spots, can include:
(i) preparing a first polymer solution and a second polymer solution differing from the first polymer solution and including a therapeutic agent;
(ii) dipping the sensor tail in the second polymer solution so that the second polymer solution coats the first region of the sensor tail but not the second region that includes the plurality of sensor spots;
   optionally drying the second polymer solution on the sensor tail to form a second polymer membrane;
(iii) dipping the sensor tail in the first polymer solution to coat the first region (e.g., which is coated by the second polymer membrane) and the second region including the plurality of sensing spots;
   optionally drying the first polymer solution on the sensor tail to form a first polymer membrane.

In certain embodiments, the method can further include repeating the dipping the sensor tail in the second polymer solution and the optionally drying the second polymer solution on the sensor tail to form the second polymer membrane until the second polymer membrane, for example, second polymer membrane 512 of FIG. 11, reaches a first set or predetermined thickness (e.g., prior to dipping the sensor tail in the first polymer solution). In certain embodiments, the method can further include repeating the dipping the sensor tail in the first polymer solution and the optionally drying the first polymer solution on the sensor tail to form the first polymer membrane until the first polymer membrane, for example, first polymer membrane 510 of FIG. 11, reaches a first set or predetermined thickness (e.g., after formation of the second polymer membrane).

In certain embodiments, the first polymer membrane can have a thickness (e.g. dry thickness) in a range of about 0.1 μm to about 1,000 μm, e.g., from about 1 μm to and about 500 μm, about 10 μm to about 300 μm, or about 10 μm to about 100 μm. In certain embodiments, the sensor tail can be dipped in the first polymer solution more than once. For example, but not by way of limitation, a sensor tail of the present disclosure can be dipped in the first polymer solution at least twice, at least three times, at least four times, at least five times, at least six times, at least seven times, at least eight times, at least nine times or at least ten times to obtain the first set or predetermined thickness. In certain embodiments, a sensor tail of the present disclosure can be dipped in the first polymer solution at least twice to obtain the first set or predetermined thickness. In certain embodiments, a sensor tail of the present disclosure can be dipped in the first polymer solution at least three times to obtain the first set or predetermined thickness. In certain embodiments, a sensor tail of the present disclosure can be dipped in the first polymer solution at least four times to obtain the first set or predetermined thickness. In certain embodiments, a sensor tail of the present disclosure can be dipped in the first polymer solution between about two times to about five times to obtain the first set or predetermined thickness. In certain embodiments, a sensor tail of the present disclosure can be dipped in the first polymer solution for a time period of about 1 second to about 5 seconds. In certain embodiments, there is a period (e.g., a wait period) of about 1-20 minutes between each dip in the first polymer solution.

In certain embodiments, the second region of the sensor tail can be ablated and/or planed prior to the dipping of the second region into the second polymer solution. For example, but not by way of limitation, a portion of the substrate and/or a portion of the working electrode can be removed from the second region by ablation and/or planing.

In certain embodiments, the second polymer membrane including a therapeutic agent can have a thickness (e.g., dry thickness) in a range of about 0.1 μm to about 1,000 μm, e.g., from about 1 μm to and about 500 μm, about 10 μm to about 500 μm, about 10 m to about 400 μm, about 10 μm to about 300 μm, about 10 μm to about 200 μm, or about 10 μm to about 100 μm. In certain embodiments, the second polymer membrane including a therapeutic agent can have a thickness in a range of about 1 μm to about 500 μm. In certain embodiments, the second polymer membrane including a therapeutic agent can have a thickness in a range of about 1 μm to about 400 μm. In certain embodiments, the second polymer membrane including a therapeutic agent can have a thickness in a range of about 1 μm to about 300 μm. In certain embodiments, the second polymer membrane including a therapeutic agent can have a thickness in a range of about 1 μm to about 200 μm. In certain embodiments, the second polymer membrane including a therapeutic agent can have a thickness in a range of about 1 μm to about 100 μm. In certain embodiments, the second polymer membrane including a therapeutic agent can have a thickness in a range of about 10 μm to about 200 μm. In certain embodiments, the second polymer membrane including a therapeutic agent can have a thickness in a range of about 10 μm to about 100 μm. In certain embodiments, the second polymer membrane including a therapeutic agent can have a thickness in a range of about 10 μm to about 300 μm. In certain embodiments, the second polymer membrane including a therapeutic agent can have a thickness in a range of about 50 μm to about 300 μm. In certain embodiments, the sensor tail can be dipped in the second polymer solution more than once. For example, but not by way of limitation, a sensor tail of the present disclosure can be dipped in the second polymer solution at least once, at least twice, at least three times, at least four times, at least five times, at least six times, at least seven times, at least eight times, at least nine times or at least ten times to obtain the second set or predetermined thickness. In certain embodiments, a sensor tail of the present disclosure can be dipped in the second polymer solution at least twice to obtain the second set or predetermined thickness. In certain embodiments, a sensor tail of the present disclosure can be dipped in the second polymer solution at least three times to obtain the second set or predetermined thickness. In certain embodiments, a sensor tail of the present disclosure can be dipped in the second polymer solution at least four times to obtain the second set or predetermined thickness. In certain embodiments, a sensor tail of the present disclosure can be dipped in the second polymer solution between about two times to about five times to obtain the second set or predetermined thickness. In certain embodiments, a sensor tail of the present disclosure can be dipped in the second polymer solution for a time period of about 1 second to about 5 seconds. In certain embodiments, there is a period (e.g., a wait period) of about 1-20 minutes between each dip in the solution.

In certain embodiments, a sensor tail of the present disclosure can be dipped in the second polymer solution at least three times to obtain a thickness of about 1 μm to about 100 μm, e.g., about 1 μm to about 50 μm.

In certain embodiments, the dipping the sensor tail in the first polymer solution can be performed before the dipping the sensor tail in the second polymer.

In certain embodiments, the dipping the sensor tail in the second polymer solution can be performed before the dipping the sensor tail in the first polymer.

In certain embodiments, the second polymer membrane can be on top of at least a portion of the first polymer membrane.

In certain embodiments, the second polymer membrane can be underneath at least a portion of the first polymer membrane.

In certain embodiments, the drying the first polymer solution on the sensor tail to form the first polymer membrane and drying the second polymer solution on the sensor tail to form the second polymer membrane can be proceeded concurrently or at the same time after the dipping the sensor tail in the first polymer solution and the dipping the sensor tail in the second polymer solution are completed.

In certain embodiments, the first polymer solution can result in the mass transport limiting membrane. In certain embodiments, the mass transport limiting membrane can be a glucose-limiting membrane. Non-limiting examples of polymers (e.g., copolymers) that can be used in a mass transport limiting membrane are disclosed in Section III herein. In certain embodiments, the first polymer can be selected from the group consisting of a polyvinylpyridine-based polymer, a polyvinylimidazole-based polymer, a poly acrylate-based polymer, a polyurethane-based polymer, a polyether urethane-based polymer, a silicone-based polymer, a derivative thereof, and combinations thereof. In certain embodiments, the first copolymer can be selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and combinations thereof. In certain embodiments, the first polymer solution can include a first copolymer selected from the group consisting of a polyvinylpyridine-based copolymer, a polyvinylimidazole-based copolymer, and combinations thereof. In certain embodiments, the polyvinylpyridine-based copolymer can be a polyvinylpyridine-co-polystyrene polymer. In certain embodiments, the first copolymer can be a polyvinylpyridine-co-polystyrene polymer, wherein the polyvinylpyridine-co-polystyrene polymer can include charged pyridine moieties. In certain embodiments, the first copolymer can include a polyvinylpyridine-co-styrene copolymer in which a portion of the pyridine nitrogen atoms are functionalized with a non-crosslinked polyethylene glycol tail and a portion of the pyridine nitrogen atoms are functionalized with an alkylsulfonic acid group, e.g., a propylsulfonic acid. In certain embodiments, a derivatized polyvinylpyridine-co-styrene copolymer for utilization as a first copolymer can be the 10Q5 polymer as described in U.S. Pat. No. 8,761,857, the content (e.g., amount) of which is incorporated by reference herein in its entirety.

In certain embodiments, the second polymer solution can include a polymer and a therapeutic agent (e.g., to form a therapeutic agent-containing polymer composition described herein). In certain embodiments, the second polymer solution can result in a drug-eluting membrane (also referred to herein as a therapeutic agent-containing polymer composition). In certain embodiments, the second polymer solution can include a polymer as described in the Section IV. For example, and not by way of limitation, the second polymer solution can include a polymer selected from a polyvinylpyridine-based polymer, a polyvinylimidazole-based polymer, a polyacrylate-based polymer, a polyurethane-based polymer, a polyether urethane-based polymer, a silicone-based polymer, a derivative thereof, and combinations thereof. In certain embodiments, the second polymer solution can include a second copolymer selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and combinations thereof.

In certain embodiments, the second polymer solution can include a polyvinylpyridine-based polymer.

In certain embodiments, the second polymer solution can include a second copolymer selected from the group consisting of a polyvinylpyridine-based copolymer, a polyvinylimidazole-based copolymer, and a combination thereof.

In certain embodiments, the second polymer solution can include a polyvinylpyridine-based copolymer.

In certain embodiments, the polyvinylpyridine-based copolymer can be a copolymer of vinylpyridine and styrene or a derivative thereof. In certain embodiments, the second copolymer can be a polyvinylpyridine-co-polystyrene polymer. In certain embodiments, the second copolymer can be poly(4-vinylpyridine-co-styrene). In certain embodiments, the polyvinylpyridine-co-polystyrene polymer can include 1-50 mer % of styrene units. In certain embodiments, the polyvinylpyridine-co-polystyrene polymer can include 1-30 mer % of styrene units. In certain embodiments, a weight averaged molecular weight of the first copolymer is in a range of about 5 kD-1,000 kD. In certain embodiments, a weight averaged molecular weight of the second copolymer is in a range of about 5 kD-1,000 kD.

In certain embodiments, an amount of the first polymer (e.g., copolymer) included in the first polymer solution can be in a range of about 1 mg/mL to about 200 mg/mL, for example, about 1 mg/mL to about 180 mg/mL, about 1 mg/mL to about 160 mg/mL, about 1 mg/mL to about 140 mg/mL, about 1 mg/mL to about 120 mg/mL, about 1 mg/mL to about 100 mg/mL, about 1 mg/mL to about 90 mg/mL, about 1 mg/mL to about 80 mg/mL, about 1 mg/mL to about 70 mg/mL, about 1 mg/mL to about 60 mg/mL, about 1 mg/mL to about 50 mg/mL, about 5 mg/mL to about 150 mg/mL, about 10 mg/mL to about 150 mg/mL, about 20 mg/mL to about 150 mg/mL, about 30 mg/mL to about 150 mg/mL, about 40 mg/mL to about 150 mg/mL, about 50 mg/mL to about 150 mg/mL, or about 60 mg/mL to about 150 mg/mL, or any values in between. In certain embodiments, an amount of the first copolymer included in the first polymer solution can be in a range of about 1 mg/mL to about 20 mg/mL, about 20 mg/mL to about 40 mg/mL, about 40 mg/mL to about 60 mg/mL, about 60 mg/mL to about 80 mg/mL, about 80 mg/mL to about 100 mg/mL, about 90 mg/mL to about 110 mg/mL, about 100 mg/mL to about 120 mg/mL, or about 120 mg/mL to about 140 mg/mL. However, embodiments of the present disclosure are not limited thereto.

In certain embodiments, the first polymer solution can further include a solvent selected from water, methanol, ethanol, propanol, isopropanol, n-butanol, iso-butanol, tetrahydrofuran, methyl-tetrahydrofuran, cyclopentyl methyl ether, and a combination thereof.

In certain embodiments, the solvent in the first polymer solution is ethanol.

In certain embodiments, an amount of the second polymer (e.g., copolymer) included in the second polymer solution can be in a range of about 1 mg/mL to about 200 mg/mL, for example, about 1 mg/mL to about 180 mg/mL, about 1 mg/mL to about 160 mg/mL, about 1 mg/mL to about 140 mg/mL, about 1 mg/mL to about 120 mg/mL, about 1 mg/mL to about 100 mg/mL, about 1 mg/mL to about 90 mg/mL, about 1 mg/mL to about 80 mg/mL, about 1 mg/mL to about 70 mg/mL, about 1 mg/mL to about 60 mg/mL, about 1 mg/mL to about 50 mg/mL, about 5 mg/mL to about 100 mg/mL, about 10 mg/mL to about 100 mg/mL, about 20 mg/mL to about 100 mg/mL, about 30 mg/mL to about 100 mg/mL, about 40 mg/mL to about 100 mg/mL, about 50 mg/mL to about 100 mg/mL, or about 60 mg/mL to about 100 mg/mL, or any values in between. In certain embodiments, an amount of the second copolymer included in the second polymer solution can be in a range of about 1 mg/mL to about 10 mg/mL, about 20 mg/mL to about 30 mg/mL, about 40 mg/mL to about 50 mg/mL, about 50 mg/mL to about 60 mg/mL, about 60 mg/mL to about 70 mg/mL, about 70 mg/mL to about 80 mg/mL, about 80 mg/mL to about 90 mg/mL, or about 90 mg/mL to about 100 mg/mL. However, embodiments of the present disclosure are not limited thereto.

In certain embodiments, the second polymer solution can further include a solvent selected from water, methanol, ethanol, propanol, isopropanol, n-butanol, iso-butanol, tetrahydrofuran, methyl-tetrahydrofuran, cyclopentyl methyl ether, and a combination thereof.

In certain embodiments, the second polymer solution can further include a solvent system comprising a solvent and a second component (e.g., a buffer). In certain embodiments, the ratio of the solvent to the second component is 8:2.

In certain embodiments, the solvent in the second polymer solution is ethanol.

In certain embodiments, a viscosity of the first polymer solution can be in a range of about 50 cP (centiPoise) to about 250 cP, for example, about 60 cP to about 250 cP, about 70 cP to about 250 cP, about 80 cP to about 250 cP, about 90 cP to about 250 cP, about 100 cP to about 250 cP, about 100 cP to about 240 cP, about 100 cP to about 230 cP, about 100 cP to about 220 cP, about 100 cP to about 210 cP, or about 100 cP to about 200 cP, or any values in between. In certain embodiments, a viscosity of the first polymer solution can be in a range of about 50 cP to about 120 cP, about 60 cP to about 130 cP, about 70 cP to about 140 cP, about 80 cP to about 150 cP, about 90 cP to about 160 cP, about 100 cP to about 170 cP, about 110 cP to about 180 cP, about 120 cP to about 190 cP, or about 130 cP to about 200 cP. In certain embodiments, the viscosity of the first polymer solution can be in a range of about 10 cP to about 150 cP, e.g., about 10 cP to about 110 cP. However, embodiments of the present disclosure are not limited thereto.

In certain embodiments, the viscosity of the first polymer solution can be in a range of about 10 cP to about 150 cP.

In certain embodiments, the viscosity of the first polymer solution can be in a range of about 10 cP to about 110 cP.

In certain embodiments, a viscosity of the second polymer solution can be in a range of about 10 cP (centiPoise) to about 250 cP, about 30 cP (centiPoise) to about 250 cP, about 50 cP (centiPoise) to about 250 cP, for example, about 60 cP to about 250 cP, about 70 cP to about 250 cP, about 80 cP to about 250 cP, about 90 cP to about 250 cP, about 100 cP to about 250 cP, about 100 cP to about 240 cP, about 100 cP to about 230 cP, about 100 cP to about 220 cP, about 100 cP to about 210 cP, or about 100 cP to about 200 cP, or any values in between. In certain embodiments, a viscosity of the second polymer solution can be in a range of about 50 cP to about 150 cP, about 60 cP to about 160 cP, about 70 cP to about 170 cP, about 80 cP to about 180 cP, about 90 cP to about 190 cP, about 100 cP to about 100 cP, about 110 cP to about 210 cP, about 120 cP to about 220 cP, or about 130 cP to about 230 cP. In certain embodiments, viscosity of the second polymer solution can be in a range of about 10 cP to about 150 cP, e.g., about 10 cP to about 110 cP. However, embodiments of the present disclosure are not limited thereto.

In certain embodiments, viscosity of the second polymer solution can be in a range of about 10 cP to about 150 cP.

In certain embodiments, viscosity of the second polymer solution can be in a range of about 10 cP to about 110 cP.

In certain embodiments, the second polymer solution (e.g., that forms the second polymer membrane) includes a therapeutic agent. In certain embodiments, the second polymer membrane is a drug-eluting membrane including a therapeutic agent (also referred to herein as a therapeutic agent-containing polymer composition). Non-limiting examples of therapeutics are described herein at Section II. Non-limiting examples of therapeutic agent-containing polymer compositions are provided in Section IV herein.

In certain embodiments, the therapeutic agent can be at least one selected from group consisting of an anti-inflammatory agent, an antiplatelet agent, an anticoagulant agent, a coagulant agent, an antiglycolytic agent, an antibiotic agent, an antiviral agent, and combinations thereof. In certain embodiments, the therapeutic agent can be an anti-inflammatory agent, an antibiotic agent, or a combination thereof. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof.

In certain embodiments, the anti-inflammatory agent can be dexamethasone or a derivative or a salt form thereof. In certain embodiments, the derivative and/or salt form of dexamethasone is dexamethasone acetate. In certain embodiments, the derivative and/or salt form of dexamethasone is dexamethasone sodium phosphate.

In certain embodiments, the therapeutic agent can be in a range of 0.01 wt %-50 wt % based on the total weight of the second polymer (e.g., in the second polymer solution).

In certain embodiments, the therapeutic agent can be in a range of 0.01 wt %-40 wt % based on the total weight of the second polymer (e.g., in the second polymer solution).

In certain embodiments, the drug-eluting membrane (also referred to as a second polymer membrane and a therapeutic agent-containing polymer composition) formed by depositing the second polymer solution can include from about 0.0005 mg to about 0.2 mg of the therapeutic agent, e.g., dexamethasone, or any values in between. In certain embodiments, the drug-eluting membrane can include about 0.0005 mg, about 0.001 mg, about 0.005 mg, about 0.01 mg, about 0.05 mg, about 0.1 mg or about 0.2 mg of the therapeutic agent, e.g., dexamethasone. In certain embodiments, the drug-eluting membrane can include from about 0.1 μg to about 20 μg of the therapeutic agent. In certain embodiments, the drug-eluting membrane can include from about 0.1 μg to about 200 μg of the therapeutic agent, e.g., from about 0.5 μg to about 200 μg, from about 1 μg to about 200 μg, from about 1.5 μg to about 200 μg, from about 2.0 μg to about 200 μg, from about 2.5 μg to about 200 μg, from about 3 μg to about 200 μg, from about 4 μg to about 200 μg, from about 5 μg to about 200 μg, from about 10 μg to about 200 μg, from about 15 μg to about 200 μg, from about 20 μg to about 200 μg, from about 25 μg to about 200 μg, from about 30 μg to about 200 μg, from about 35 μg to about 200 μg, from about 40 μg to about 200 μg, from about 45 μg to about 200 μg, from about 50 μg to about 200 μg, from about 55 μg to about 200 μg, from about 60 μg to about 200 μg, from about 65 μg to about 200 μg, from about 70 μg to about 200 μg, from about 75 μg to about 200 μg, from about 80 μg to about 200 μg, from about 85 μg to about 200 μg, from about 90 μg to about 200 μg, from about 95 μg to about 200 μg, from about 100 μg to about 200 μg, from about 110 μg to about 200 μg, from about 120 μg to about 200 μg, from about 130 μg to about 200 μg, from about 140 μg to about 200 μg, from about 150 μg to about 200 μg, from about 160 μg to about 200 μg, from about 170 μg to about 200 μg, from about 180 μg to about 200 μg, from about 190 μg to about 200 μg, from about 0.1 μg to about 190 μg, from about 0.1 μg to about 180 μg, from about 0.1 μg to about 170 μg, from about 0.1 μg to about 160 μg, from about 0.1 μg to about 150 μg, from about 0.1 μg to about 140 μg, from about 0.1 μg to about 130 μg, from about 0.1 μg to about 120 μg, from about 0.1 μg to about 110 μg, from about 0.1 μg to about 100 μg, from about 1 μg to about 150 μg, from about 5 μg to about 150 μg or from about 5 μg to about 120 μg. In certain embodiments, the drug-eluting membrane formed by depositing the second polymer solution can include from about 1 μg to about 100 μg of the therapeutic agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In certain embodiments, the drug-eluting membrane can include from about 1 μg to about 5 μg of the therapeutic agent. In certain embodiments, the drug-eluting membrane can include from about 1 μg to about 10 μg of the therapeutic agent. In certain embodiments, the drug-eluting membrane can include from about 1 μg to about 15 μg of the therapeutic agent. In certain embodiments, the drug-eluting membrane can include from about 1 μg to about 20 μg of the therapeutic agent. In certain embodiments, the drug-eluting membrane can include from about 5 μg to about 20 μg of the therapeutic agent. In certain embodiments, the drug-eluting membrane can include from about 1 μg to about 30 μg of the therapeutic agent. In certain embodiments, the drug-eluting membrane can include from about 5 μg to about 10 μg of the therapeutic agent. In certain embodiments, the drug-eluting membrane can include from about 5 μg to about 15 μg of the therapeutic agent. In certain embodiments, the drug-eluting membrane can include from about 5 μg to about 20 μg of the therapeutic agent. In certain embodiments, the drug-eluting membrane can include from about 5 μg to about 25 μg of the therapeutic agent. In certain embodiments, the drug-eluting membrane can include from about 5 μg to about 30 μg of the therapeutic agent. In certain embodiments, the drug-eluting membrane can include from about 0.1 μg to about 30 μg of the therapeutic agent. In certain embodiments, the drug-eluting membrane can include from about 0.1 μg to about 20 μg of the therapeutic agent. In certain embodiments, the drug-eluting membrane can include from about 0.1 μg to about 15 μg of the therapeutic agent. In certain embodiments, the drug-eluting membrane can include from about 0.1 μg to about 10 μg of the therapeutic agent. In certain embodiments, the drug-eluting membrane can include from about 0.1 μg to about 5 μg of the therapeutic agent.

In certain embodiments, the drug-eluting membrane can include from about 0.01 μg to about 5 μg of the therapeutic agent.

In certain embodiments, the drug-eluting membrane can include less than about 10 μg of the therapeutic agent.

In certain embodiments, the drug-eluting membrane can include less than about 5 μg of the therapeutic agent.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.001 μg/day to about 1 mg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.001 μg/day to about 5 μg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.001 μg/day to about 1 μg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.001 μg/day to about 0.1 μg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.01 μg/day to about 100 μg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.01 μg/day to about 10 μg/day of the therapeutic agent, e.g., dexamethasone, or any values in between.

In certain embodiments, the drug-eluting membrane (also referred to as a second polymer membrane and a therapeutic agent-containing polymer composition) formed by depositing the second polymer solution can continuously release the therapeutic agent at a drug delivery rate of about 0.01 μg/day to about 5 μg/day of the therapeutic agent, e.g., dexamethasone, or any values in between.

In certain embodiments, the drug-eluting membrane formed by depositing the second polymer solution can continuously release the therapeutic agent at a drug delivery rate of about 0.01 μg/day to about 1 mg/day of the therapeutic agent, e.g., dexamethasone, or any values in between. In certain embodiments, the drug-eluting membrane can continuously release the therapeutic agent at a drug delivery rate of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the therapeutic agent, e.g., dexamethasone, or any values in between. In certain embodiments, the drug-eluting membrane can continuously release the therapeutic agent at a drug delivery rate of about 0.2 μg/day to about 5 μg/day of the therapeutic agent, e.g., dexamethasone. In certain embodiments, the drug-eluting membrane can continuously release the therapeutic agent at a drug delivery rate of about 0.2 μg/day to about 2 μg/day of the therapeutic agent, e.g., dexamethasone. In certain embodiments, the drug-eluting membrane can continuously release the therapeutic agent at a drug delivery rate of about 0.2 μg/day to about 1 μg/day of the therapeutic agent, e.g., dexamethasone. In certain embodiments, the drug-eluting membrane can continuously release the therapeutic agent at a set or predetermined drug delivery rate to achieve desirable therapeutic results such as reducing, minimizing, preventing, and/or inhibiting the inflammation.

In certain embodiments, the drug-eluting membrane (also referred to as a second polymer membrane and a therapeutic agent-containing polymer composition) can continuously release the therapeutic agent at a set or predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days.

In certain embodiments, the drug-eluting membrane (also referred to as a second polymer membrane and a therapeutic agent-containing polymer composition) can continuously release the therapeutic agent at a set or predetermined drug delivery rate for a set or predetermined days such as for at least 30 days. Additional features of the drug-eluting membrane (also referred to herein as therapeutic agent-containing polymer compositions) are disclosed herein at Section IV.

One or more aspects of embodiments of the present disclosure are directed toward methods for depositing a therapeutic agent-containing polymer composition on a structure or component of an analyte sensor. Early-stage trials highlighted issues of reduced performance and ascorbic acid interference over time due to increased diffusion of the dexamethasone drug out of the drug-eluting membrane. Further, the polymer of choice of the drug-eluting membrane for controlled or selected drug elution has high hydrophobic character which is unsuitable for covering the sensing chemistry of the analyte sensor, for example, glucose sensing chemistry. This poses an issue for deposition techniques, as the only portion of the analyte sensor with a large area not coated with the sensing chemistry is the counter electrode opposite to the working electrode of the analyte sensor, as shown in FIGS. 1A-1B and FIG. 2, on the underside of the analyte sensor. Underside sensor deposition, in tandem with the relatively large deposition volume required, makes a high-volume adoption of a dispensing process difficult.

In certain embodiments, a therapeutic agent-containing polymer composition of the present disclosure can be deposited onto a structure of the sensor tail. For example, but not by way of limitation, depositing a therapeutic agent-containing polymer composition of the present disclosure onto a structure or component of the sensor tail can be performed by dipping, spraying, aerosol deposition, micro-dispensing, jetting, drop-casting, etc. In certain embodiments, depositing a therapeutic agent-containing polymer composition of the present disclosure onto a structure of the sensor tail can be performed by dipping. In certain embodiments, depositing a therapeutic agent-containing polymer composition of the present disclosure onto a structure or component of the sensor tail can be performed by drop-casting. In certain embodiments, depositing a therapeutic agent-containing polymer composition of the present disclosure onto a structure of the sensor tail can be performed by aerosol deposition. For example, but not by way of limitation, a therapeutic agent-containing polymer composition can be disposed upon a surface of an electrode (e.g., a counter/reference electrode (e.g., 216 of FIG. 17A) and/or a working electrode (e.g., 214 of FIG. 17A)), an insulating material (e.g., a dielectric material (e.g., 219a-c of FIG. 17A)), a substrate (e.g., 212 of FIG. 17A) and/or a mass transport limiting membrane (e.g., 220 of FIG. 17A). In certain embodiments, a therapeutic agent-containing polymer composition is not disposed upon the active layer (e.g., the multiple sensing spots). In certain embodiments, a therapeutic agent-containing polymer composition is disposed upon a structure or component of the analyte sensor that is adjacent to the active layer (e.g., the multiple sensing spots).

The present disclosure further provides methods for generating a therapeutic agent-containing polymer composition described herein on a structure of an analyte sensor, e.g., an electrode, a substrate and/or an insulating material. In certain embodiments, a therapeutic agent-containing polymer composition of the present disclosure can be generated on an electrode, e.g., a counter electrode, of an analyte sensor described herein. In certain embodiments, a therapeutic agent-containing polymer composition of the present disclosure can be generated on an electrode, e.g., a counter electrode, of an analyte sensor described herein. For example, but not by way of limitation, a therapeutic agent-containing polymer composition of the present disclosure can be generated as one or more spots onto a structure of an analyte sensor, e.g., an electrode, a substrate and/or an insulating material.

In certain embodiments, a method for manufacturing an analyte sensor comprising a therapeutic agent can include:
(a) providing an analyte sensor comprising (i) a working electrode, (ii) a counter and/or a reference electrode and (iii) an active area;
(b) preparing a first polymer solution comprising a first polymer or providing a first polymer solution comprising a first polymer;
(c) preparing a second polymer solution comprising a second polymer and a therapeutic agent or providing a second polymer solution comprising a second polymer and a therapeutic agent;
(d) depositing the second polymer solution on at least a portion of the counter and/or a reference electrode to generate a therapeutic agent-containing polymer composition; and
(e) depositing the first polymer solution on the in vivo portion to generate a first polymer membrane on top at least a portion of the therapeutic agent-containing polymer composition.

In certain embodiments, a method for fabricating an analyte sensor comprising a therapeutic agent can include:
(a) patterning a plurality of first conductive layers on a substrate to generate a plurality of working electrodes;
(b) patterning a plurality of second conductive layers on the substrate to generate a plurality of counter and/or reference electrodes;
(c) forming one or more spots of a therapeutic agent-containing polymer composition on each counter and/or reference electrode of the plurality of counter and/or reference electrodes, wherein the therapeutic agent-containing polymer composition comprises a polymer and a therapeutic agent; and
(d) singulating individual analyte sensors from the substrate, wherein each individual analyte sensor comprises at least one working electrode and at least one counter and/or reference electrode.

In certain embodiments, forming the one or more spots of a therapeutic agent-containing polymer composition on each of the plurality of counter and/or reference electrodes comprising dispensing two or more layers of a polymer solution comprising the therapeutic agent. In certain embodiments, at least one of the two or more spots of a therapeutic agent-containing polymer composition is formed by dispensing two layers of the polymer solution and a second spot of the two or more spots of a therapeutic agent-containing polymer composition is formed by dispensing three layers of the polymer solution.

In certain embodiments, depositing the second polymer solution on at least a portion of the counter and/or reference electrode comprises a screen printing, a rotary printing, a jetting, an aerosol deposition, micro-dispensing, a spray coating, a dip coating, a drop-casting, a spin coating or a brush coating process.

In certain embodiments, depositing the first polymer solution on the in vivo portion comprises a screen printing, a rotary printing, micro-dispensing, a jetting, an aerosol deposition, a spray coating, a dip coating, a drop-casting, a spin coating or a brush coating process.

In certain embodiments, depositing the second polymer solution on the counter and/or reference electrode comprising depositing the second polymer solution onto the counter and/or reference electrode as two or more spots of the therapeutic agent-containing polymer composition.

In certain embodiments, the at least two of the two or more spots comprise different amounts of the therapeutic agent. In certain embodiments, at least one of the spots has a circle shape, an oval shape, a regular polygon shape or an irregular polygon shape.

In certain embodiments, a therapeutic agent-containing polymer composition of the present disclosure can be generated as one or more spots onto a structure of an analyte sensor, e.g., an electrode, a substrate and/or an insulating material. In certain embodiments, 1 or more spots, 2 or more spots, 3 or more spots, 4 or more spots, 5 or more spots, 6 or more spots, 7 or more spots, 8 or more spots, 9 or more spots, 10 or more spots, 11 or more spots, 12 or more spots, 13 or more spots, 14 or more spots, 15 or more spots, 16 or more spots, 17 or more spots, 18 or more spots, 19 or more spots, 20 or more spots, 21 or more spots, 22 or more spots, 23 or more spots, 24 or more spots, 25 or more spots, 26 or more spots, 27 or more spots, 28 or more spots, 29 or more spots or 30 or more spots of a therapeutic agent-containing polymer composition can be deposited onto a structure of an analyte sensor, e.g., an electrode, a substrate and/or an insulating material.

In certain embodiments, about 1 to about 30 spots of a therapeutic agent-containing polymer composition can be deposited onto a structure of an analyte sensor, e.g., an electrode, a substrate and/or an insulating material. In certain embodiments, about 1 to about 10 spots of a therapeutic agent-containing polymer composition can be deposited onto a structure of an analyte sensor, e.g., an electrode, a substrate and/or an insulating material.

In certain embodiments, a therapeutic agent-containing polymer composition of the present disclosure can be generated as one or more spots onto an electrode, e.g., a counter electrode. In certain embodiments, 1 or more spots, 2 or more spots, 3 or more spots, 4 or more spots, 5 or more spots, 6 or more spots, 7 or more spots, 8 or more spots, 9 or more spots, 10 or more spots, 11 or more spots, 12 or more spots, 13 or more spots, 14 or more spots, 15 or more spots, 16 or more spots, 17 or more spots, 18 or more spots, 19 or more spots, 20 or more spots, 21 or more spots, 22 or more spots, 23 or more spots, 24 or more spots, 25 or more spots, 26 or more spots, 27 or more spots, 28 or more spots, 29 or more spots or 30 or more spots of a therapeutic agent-containing polymer composition can be deposited onto a counter electrode of an analyte sensor described herein. In certain embodiments, about 1 to about 30 spots of a therapeutic agent-containing polymer composition can be deposited onto a counter electrode of an analyte sensor described herein. In certain embodiments, about 1 to about 10 spots of a therapeutic agent-containing polymer composition can be deposited onto a counter electrode of an analyte sensor described herein.

In certain embodiments, the one or more spots, e.g., two or more spots, can have the shape of a circle, can have the shape of an oval, can have the shape of a regular polygon or can have the shape of an irregular polygon. In certain embodiments, each of the spots have the same or similar shapes. For example, but not by way of limitation, each of the spots can have a circular or rounded shape. In certain embodiments, at least two or more of the spots have different shapes, e.g., one of the spots can be circular or oval in shape and a different spot can have the shape of a regular or irregular polygon.

In certain embodiments, each spot contains the same or similar amount of the therapeutic agent. Alternatively, at least one of the spots contains a different amount of the therapeutic agent compared to one other spot. In certain embodiments, at least two or more of the spots have a difference in the amount of therapeutic agent of about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 105% or more, about 110% or more, about 115% or more, about 120% or more, about 125% or more, about 130% or more, about 135% or more, about 140% or more, about 145% or more, about 150% or more, about 155% or more, about 160% or more, about 165% or more, about 170% or more, about 180% or more, about 185% or more, about 190% or more, about 195% or more or 200% or more. In certain embodiments, at least two or more of the spots have a difference in the amount of therapeutic agent of about 50% or more. In certain embodiments, at least two or more of the spots have a difference in the amount of therapeutic agent of about 60% or more. In certain embodiments, at least two or more of the spots have a difference in the amount of therapeutic agent of about 70% or more. In certain embodiments, at least two or more of the spots have a difference in the amount of therapeutic agent of about 80% or more. In certain embodiments, at least two or more of the spots have a difference in the amount of therapeutic agent of about 90% or more. In certain embodiments, at least two or more of the spots have a difference in the amount of therapeutic agent of about 100% or more.

In certain embodiments, each spot has the same or similar thickness. Alternatively, at least two or more of the spots differ in thicknesses, e.g., as shown in FIG. 12A and FIG. 12B. In certain embodiments, at least two or more of the spots have a difference in thickness of about 5% or more, about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 100% or more, about 105% or more, about 110% or more, about 115% or more, about 120% or more, about 125% or more, about 130% or more, about 135% or more, about 140% or more, about 145% or more, about 150% or more, about 155% or more, about 160% or more, about 165% or more, about 170% or more, about 180% or more, about 185% or more, about 190% or more, about 195% or more or 200% or more. For example, but not by way of limitation, at least two or more of the spots have a difference in thickness of about 50% or more. In certain embodiments, at least two or more of the spots have a difference in thickness of about 60% or more. In certain embodiments, at least two or more of the spots have a difference in thickness of about 70% or more. In certain embodiments, at least two or more of the spots have a difference in thickness of about 80% or more. In certain embodiments, at least two or more of the spots have a difference in thickness of about 90% or more. In certain embodiments, at least two or more of the spots have a difference in thickness of about 100% or more.

In certain embodiments, at least two of the two or more spots differ in thickness, e.g., where at least two of the two or more spots differ in thickness by more than about 50% (e.g., by more than about 70%). In certain embodiments, the difference in thickness is obtained by depositing more layers on one spot compared to another spot. As shown in FIG. 12B, an exemplary sensor tail of the present disclosure can comprise one spot of a therapeutic agent-containing polymer composition that includes two layers (e.g., two deposition layers) and can comprise a second that includes three layers (e.g., three deposition layers). In certain embodiments, the thickness of each layer and the amount of therapeutic agent in each layer is the same or similar. In certain embodiments, each spot can be obtained by depositing between about one to about ten layers (e.g., about one to about five layers or about one to about three layers) of a therapeutic agent-containing polymer composition onto a structure of the sensor tail, e.g., on a counter electrode of a sensor tail.

The monitoring, detection, manufacturing and/or any other relevant devices or components according to embodiments of the present invention described herein can be implemented utilizing any suitable hardware, firmware (e.g., an application-specific integrated circuit), software, or a combination of software, firmware, and hardware. For example, the various components of a device can be formed on one integrated circuit (IC) chip or on separate IC chips. Further, the various components of the device can be implemented on a flexible printed circuit film, a tape carrier package (TCP), a printed circuit board (PCB), or formed on one substrate. Further, the various components of the device can be a process or thread, running on one or more processors, in one or more computing devices, executing computer program instructions and interacting with other system components for performing the various functionalities described herein. The computer program instructions are stored in a memory which can be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices can be combined or integrated into a single computing device, or the functionality of a particular computing device can be distributed across one or more other computing devices without departing from the scope of the embodiments of the present disclosure.

### VII. Insertion Devices

The present disclosure further provides a sharp for inserting an analyte sensor into a subject. For example, but not by way of limitation, the present disclosure provides a sharp that is coated with a therapeutic agent-containing polymer composition. In certain embodiments, the present disclosure further provides an insertion device that includes a sharp coated with a therapeutic agent-containing polymer composition and analyte sensor.

In certain embodiments, a sharp of the present disclosure is coated with a therapeutic agent-containing polymer composition. Non-limiting examples of therapeutic agents that can be incorporated into a therapeutic agent-containing polymer composition coating a sharp are disclosed in Section II. Non-limiting examples of therapeutic agent-containing polymer compositions that can be used to coat a sharp of the present disclosure are described in Section IV.

In certain embodiments, a therapeutic agent-containing polymer composition for coating a sharp can include a therapeutic agent selected from group consisting of an antibiotic agent, an antiviral agent, an anti-inflammatory agent, an anti-cancer agent, an antiplatelet agent, an anticoagulant agent, a coagulant agent, an antiglycolytic agent and combinations thereof. In certain embodiments, the therapeutic agent is an antibiotic agent. In certain embodiments, the therapeutic agent is an antiviral agent. In certain embodiments, the therapeutic agent is an anti-inflammatory agent. In certain embodiments, the therapeutic agent is an anti-cancer agent. In certain embodiments, the therapeutic agent is an antiplatelet agent. In certain embodiments, the therapeutic agent is an anticoagulant agent. In certain embodiments, the therapeutic agent is a coagulant agent. In certain embodiments, the therapeutic agent is an antiglycolytic agent.

In certain embodiments, the therapeutic agent can be an anti-inflammatory agent. In certain embodiments, the anti-inflammatory agent is a non-steroidal anti-inflammatory agent. In certain embodiments, the anti-inflammatory agent is a steroidal anti-inflammatory agent, e.g., a corticosteroid. In certain embodiments, the anti-inflammatory agent can be one or more selected from among triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and a derivative or salt forms thereof. Non-limiting salt forms include pharmaceutically acceptable salts including acetate and phosphate salts. In certain embodiments, the anti-inflammatory agent is a salt of dexamethasone.

In certain embodiments, the anti-inflammatory agent is dexamethasone or a derivative or a salt form thereof.

In certain embodiments, the anti-inflammatory agent is dexamethasone acetate.

In certain embodiments, the anti-inflammatory agent is dexamethasone sodium phosphate.

In certain embodiments, a therapeutic agent-containing polymer composition for coating a sharp can include an amount of the therapeutical agent, e.g., dexamethasone or a derivative thereof, in a range of 0.01 wt %-50 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 0.01 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 1 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 5 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 10 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 20 wt %-40 wt % based on the total weight of the therapeutic agent-containing polymer composition. In certain embodiments, the therapeutic agent-containing polymer composition can include an amount of the therapeutical agent, e.g., dexamethasone or a derivative thereof, in a range of 30 wt %-40 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 5 wt %-20 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 5 wt %-10 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-10 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-20 wt % based on the total weight of the therapeutic agent-containing polymer composition. In certain embodiments, the therapeutic agent-containing polymer composition can include an amount of the therapeutic agent, e.g., dexamethasone or a derivative thereof, in a range of about 1 wt %-30 wt % based on the total weight of the therapeutic agent-containing polymer composition.

In certain embodiments, the therapeutic agent-containing polymer composition can include an amount of the therapeutic agent, e.g., dexamethasone or a derivative thereof, in a range of about 10 wt %-20 wt % based on the total weight of the therapeutic agent-containing polymer composition.

In certain embodiments, a therapeutic agent-containing polymer composition for coating a sharp can include from about 0.0005 mg to about 0.2 mg of the therapeutic agent, e.g., dexamethasone, or any values in between. In certain embodiments, the therapeutic agent-containing polymer composition can include about 0.0005 mg, about 0.001 mg, about 0.005 mg, about 0.01 mg, about 0.05 mg, about 0.1 mg or about 0.2 mg of the therapeutic agent, e.g., dexamethasone. In certain embodiments, the therapeutic agent-containing polymer composition can include from about 0.0005 mg to about 0.1 mg, about 0.0005 mg to about 0.05 mg, about 0.0005 mg to about 0.01 mg, about 0.0005 mg to about 0.005 mg or about 0.0005 mg to about 0.001 mg of the therapeutic agent, e.g., dexamethasone. In certain embodiments, the therapeutic agent-containing polymer composition can include from about 0.0005 mg to about 0.1 mg. In certain embodiments, the therapeutic agent-containing polymer composition can include from about 0.0005 mg to about 0.01 mg. In certain embodiments, the therapeutic agent-containing polymer composition can include from about 0.001 mg to about 0.1 mg. In certain embodiments, the therapeutic agent-containing polymer composition can include from about 0.001 mg to about 0.01 mg. In certain embodiments, the therapeutic agent-containing polymer composition can include from about 0.001 mg to about 0.005 mg. In certain embodiments, the therapeutic agent-containing polymer composition can include from about 0.001 mg to about 0.003 mg. In certain embodiments, the therapeutic agent-containing polymer composition can include from about 0.1 μg to about 200 μg of the therapeutic agent, e.g., from about 0.5 μg to about 200 μg, from about 1 μg to about 200 μg, from about 1.5 μg to about 200 μg, from about 2.0 μg to about 200 μg, from about 2.5 μg to about 200 μg, from about 3 μg to about 200 μg, from about 4 μg to about 200 μg, from about 5 μg to about 200 μg, from about 10 μg to about 200 μg, from about 15 μg to about 200 μg, from about 20 μg to about 200 μg, from about 25 μg to about 200 μg, from about 30 μg to about 200 μg, from about 35 μg to about 200 μg, from about 40 μg to about 200 μg, from about 45 μg to about 200 μg, from about 50 μg to about 200 μg, from about 55 μg to about 200 μg, from about 60 μg to about 200 μg, from about 65 μg to about 200 μg, from about 70 μg to about 200 μg, from about 75 μg to about 200 μg, from about 80 μg to about 200 μg, from about 85 μg to about 200 μg, from about 90 μg to about 200 μg, from about 95 μg to about 200 μg, from about 100 μg to about 200 μg, from about 110 μg to about 200 μg, from about 120 μg to about 200 μg, from about 130 μg to about 200 μg, from about 140 μg to about 200 μg, from about 150 μg to about 200 μg, from about 160 μg to about 200 μg, from about 170 μg to about 200 μg, from about 180 μg to about 200 μg, from about 190 μg to about 200 μg, from about 0.1 μg to about 190 μg, from about 0.1 μg to about 180 μg, from about 0.1 μg to about 170 μg, from about 0.1 μg to about 160 μg, from about 0.1 μg to about 150 μg, from about 0.1 μg to about 140 μg, from about 0.1 μg to about 130 μg, from about 0.1 μg to about 120 μg, from about 0.1 μg to about 110 μg, from about 0.1 μg to about 100 μg, from about 1 μg to about 150 μg, from about 5 μg to about 150 μg or from about 5 μg to about 120 μg. In certain embodiments, the therapeutic agent-containing polymer composition can include from about 0.1 μg to about 20 μg of the therapeutic agent. In certain embodiments, the therapeutic agent-containing polymer composition can include from about 1 μg to about 100 μg of the therapeutic agent, e.g., from about 1 μg to about 95 μg, from about 1 μg to about 90 μg, from about 1 μg to about 85 μg, from about 1 μg to about 80 μg, from about 1 μg to about 75 μg, from about 1 μg to about 70 μg, from about 1 μg to about 65 μg, from about 1 μg to about 60 μg, from about 1 μg to about 55 μg, from about 1 μg to about 50 μg, from about 1 μg to about 45 μg, from about 1 μg to about 40 μg, from about 1 μg to about 35 μg, from about 1 μg to about 30 μg, from about 1 μg to about 25 μg, from about 1 μg to about 20 μg, from about 1 μg to about 15 μg, from about 1 μg to about 14 μg, from about 1 μg to about 13 μg, from about 1 μg to about 12 μg, from about 1 μg to about 11 μg, from about 1 μg to about 10 μg, from about 1 μg to about 9 μg, from about 2 μg to about 100 μg, from about 3 μg to about 100 μg, from about 4 μg to about 100 μg, from about 5 μg to about 100 μg, from 5 about 6 μg to about 100 μg, from about 7 μg to about 100 μg, from about 8 μg to about 100 μg, from about 9 μg to about 100 μg, from about 10 μg to about 100 μg, from about 11 μg to about 100 μg, from about 12 μg to about 100 μg, from about 13 μg to about 100 μg, from about 14 μg to about 100 μg, from about 15 μg to about 100 μg, from about 16 μg to about 100 μg, from about 17 μg to about 100 μg, from about 18 μg to about 100 μg, from about 19 μg to about 100 μg, from about 20 μg to about 100 μg, from about 25 μg to about 100 μg, from about 30 μg to about 100 μg, from about 35 μg to about 100 μg, from about 40 μg to about 100 μg, from about 45 μg to about 100 μg, from about 50 μg to about 100 μg, from about 55 μg to about 100 μg, from about 60 μg to about 100 μg, from about 65 μg to about 100 μg, from about 70 μg to about 100 μg, from about 75 μg to about 100 μg, from about 80 μg to about 100 μg, from about 85 μg to about 100 μg, from about 90 μg to about 100 μg, from about 95 μg to about 100 μg, from about 5 μg to about 50 μg, from about 5 μg to about 45 μg, from about 5 μg to about 40 μg, from about 5 μg to about 35 μg, from about 5 μg to about 30 μg, from about 5 μg to about 25 μg, or from about 5 μg to about 20 μg. In certain embodiments, the therapeutic agent-containing polymer composition can include from about 1 μg to about 20 μg of the therapeutic agent. In certain embodiments, the therapeutic agent-containing polymer composition can include from about 5 μg to about 20 μg of the therapeutic agent. In certain embodiments, the therapeutic agent-containing polymer composition can include from about 1 μg to about 30 μg of the therapeutic agent. In certain embodiments, the therapeutic agent-containing polymer composition can include from about 5 μg to about 30 μg of the therapeutic agent. In certain embodiments, the therapeutic agent-containing polymer composition can include from about 0.1 μg to about 15 μg of the therapeutic agent. In certain embodiments, the therapeutic agent-containing polymer composition can include from about 0.1 μg to about 10 μg of the therapeutic agent.

In certain embodiments, the therapeutic agent-containing polymer composition can include from about 0.1 μg to about 5 μg of the therapeutic agent.

In certain embodiments, a therapeutic agent-containing polymer composition for coating a sharp can continuously release the therapeutic agent at a drug delivery rate (e.g., an average drug delivery rate) of about 0.001 μg/day to about 1 mg/day of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.001 μg/day to about 5 μg/day of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.001 μg/day to about 1 μg/day of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.001 μg/day to about 0.1 μg/day of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.01 μg/day to about 100 μg/day of the therapeutic agent of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.01 μg/day to about 10 μg/day of the therapeutic agent of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.01 μg/day to about 5 μg/day of the therapeutic agent of the therapeutic agent, e.g., dexamethasone, or any values in between.

In certain embodiments, a therapeutic agent-containing polymer composition for coating a sharp can continuously release the therapeutic agent at a drug delivery rate (e.g., an average drug delivery rate) of about 0.01 μg/day to about 1 mg/day of the therapeutic agent, e.g., dexamethasone, or any values in between.

In certain embodiments, the therapeutic agent-containing polymer composition can continuously release the therapeutic agent at a drug delivery rate (e.g., an average drug delivery rate) of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 20 μg/day, about 30 μg/day, about 40 μg/day, about 50 μg/day, about 60 μg/day, about 70 μg/day, about 80 μg/day, about 90 μg/day, about 100 μg/day, about 200 μg/day, about 300 μg/day, about 400 μg/day, about 500 μg/day, about 600 μg/day, about 700 μg/day, about 800 μg/day, about 900 μg/day, or about 1 mg/day of the therapeutic agent, e.g., dexamethasone, or any values in between. In certain embodiments, the therapeutic agent-containing polymer composition can continuously release the therapeutic agent at a drug delivery rate (e.g., an average drug delivery rate) of about 0.2 μg/day to about 5 μg/day of the therapeutic agent, e.g., dexamethasone. In certain embodiments, the therapeutic agent-containing polymer composition can continuously release the therapeutic agent at a drug delivery rate (e.g., an average drug delivery rate) of about 0.2 μg/day to about 2 μg/day of the therapeutic agent, e.g., dexamethasone. In certain embodiments, the therapeutic agent-containing polymer composition can continuously release the therapeutic agent at a drug delivery rate (e.g., an average drug delivery rate) of about 0.2 μg/day to about 1 μg/day of the therapeutic agent, e.g., dexamethasone. In certain embodiments, the therapeutic agent-containing polymer composition can continuously release the therapeutic agent at a set or predetermined drug delivery rate to achieve desirable therapeutic results such as reducing, minimizing, preventing, and/or inhibiting the inflammation and/or infection. In certain embodiments, the therapeutic agent-containing polymer composition can continuously release the therapeutic agent at a set or predetermined drug delivery rate to reduce and/or minimize sensor signal inaccuracies or in vivo sensor failure, e.g., due to FBR. In certain embodiments, the therapeutic agent-containing polymer composition can continuously release the therapeutic agent at a set or predetermined drug delivery rate to achieve desirable therapeutic results such as minimizing and/or reducing LSA.

In certain embodiments, a therapeutic agent-containing polymer composition for coating a sharp can continuously release the therapeutic agent at a set or predetermined drug delivery rate for at least 1 day, for at least 2 days, for at least 3 days, for at least 4 days, for at least 5 days, for at least 6 days, for at least 7 days, for at least 8 days, for at least 9 days, for at least 10 days, for at least 11 days, for at least 12 days, for at least 13 days, for at least 14 days, for at least 15 days, for at least 16 days, for at least 17 days, for at least 18 days, for at least 19 days, for at least 20 days, for at least 21 days, for at least 22 days, for at least 23 days, for at least 24 days, for at least 25 days, for at least 26 days, for at least 27 days, for at least 28 days, for at least 29 days, or for at least 30 days. In certain embodiments, the therapeutic agent-containing polymer composition can continuously release the therapeutic agent at a set or predetermined drug delivery rate for a set or predetermined days such as for at least 30 days.

In certain embodiments, the therapeutic agent-containing polymer composition is configured to release a therapeutic agent into the tissue of a wearer over a period of at least 7 days. In certain embodiments, the therapeutic agent-containing polymer composition provides controlled release of the therapeutic agent into the tissue of a wearer over a period of at least 7 days. In certain embodiments, the therapeutic agent-containing polymer composition provides controlled release of the therapeutic agent into the tissue of a wearer over a period of at least 14 days. In certain embodiments, the therapeutic agent-containing polymer composition provides controlled release of the therapeutic agent in the tissue of a wearer over a period of at least 30 days. In certain embodiments, the therapeutic agent-containing polymer composition provides controlled release of the therapeutic agent into the tissue of a wearer over a period of at least 4 days, at least 5 days, at least 7 days, at least 10 days, at least 14 days, at least 20 days, or at least 30 days.

In certain embodiments, a therapeutic agent-containing polymer composition for coating a sharp can include a polymer selected from a polyvinylpyridine-based polymer, a polyvinylimidazole-based polymer, a polyacrylate-based polymer, a polyurethane-based copolymer, a polyether urethane-based polymer, a silicone-based polymer, a derivative thereof, and combinations thereof. In certain embodiments, the polymer in the therapeutic agent-containing polymer composition that is used to a coat a sharp of the present disclosure can be a biodegradable or bioresorbable polymer, such as but not limited to polycaprolactone (PCL), poly(glycolic acid) (PGA), poly(lactic acid) (PLA) or poly(D,L-lactide-co-glycolide) (PLGA). In certain embodiments, the polymer can be a polylactide, a polyglycolide or polyethylene glycol polymer. In certain embodiments, the polymer can be a blend of two or three of these functionalities as a block copolymer, e.g., a diblock copolymer or a triblock copolymer. Non-limiting embodiments of such block copolymers include poly(D,L-lactic-co-glycolic acid) (PLGA) and triblock copolymer polylactide-block-poly(ethylene glycol)-block-polylactide (PLA-PEG-PLA). Additional non-limiting examples of block copolymers include PEO and PPO copolymers such as PEO-PPO diblock copolymers, PPO-PEO-PPO triblock copolymers, PEO-PPO-PEO triblock copolymers, alternating block copolymers of PEO-PPO, random copolymers of ethylene oxide and propylene oxide and blends thereof. In certain embodiments, the polymer is polycaprolactone (PCL) or a derivative thereof.

In certain embodiments, the polymer is poly(glycolic acid) (PGA) or a derivative thereof.

In certain embodiments, the polymer is poly(lactic acid) (PLA) or a derivative thereof.

In certain embodiments, the polymer is poly(D,L-lactide-co-glycolide) (PLGA) or a derivative thereof.

A therapeutic agent-containing polymer composition can be placed on any portion or component of the sharp described herein. For example, but not by way of limitation, the therapeutic agent-containing polymer composition can be applied to the distal tip of the sharp. As used herein, the distal portion of the sharp refers to a portion of the sharp that is located away from the insertion site on the skin surface and extends to the insertion tip of the sharp. As used herein, the term “insertion tip” refers to the bottom portion of the sharp that allows the sharp to be inserted into a skin surface. As used herein, the proximal portion of the sharp refers to a portion of the sharp that is closest to the insertion site on the skin surface.

Alternatively, the entire length of the sharp can be coated with a therapeutic agent-containing polymer composition.

In certain embodiments, at least a portion of the interior surface of the sharp (e.g., the surface of the channel of the sharp) is coated with a therapeutic agent-containing polymer composition.

In certain embodiments, at least a portion of the exterior surface of the sharp is coated with a therapeutic agent-containing polymer composition.

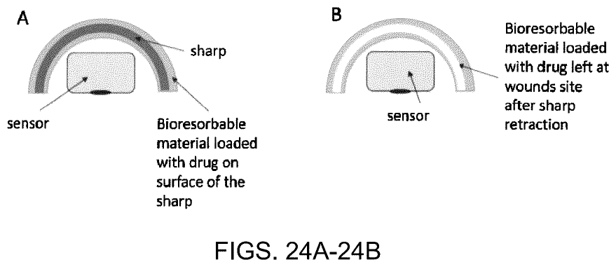

In certain embodiments, at least a portion of both the interior and exterior surfaces of the sharp are coated with a therapeutic agent-containing polymer composition, e.g., as shown in FIGS. 24A-24B.

In certain embodiments, the therapeutic agent-containing polymer composition coating the insertion tip is detachably attached to the insertion tip. For example, but not by way of limitation, upon removal of the insertion tip from the subject, the therapeutic agent-containing polymer composition is retained in the subject, e.g., in close proximity to the analyte sensor that was inserted into the subject by the sharp.

In certain embodiments, the insertion tip of a sharp is coated with a therapeutic agent-containing polymer composition. In certain embodiments, from about 50% to about 100% of the distal portion of the sharp between the tip of the sharp and the proximal portion of the sharp at the skin surface is coated with a therapeutic agent-containing polymer composition. In certain embodiments, from about 50% to about 100%, from about 50% to about 80%, from about 50% to about 60%, from about 60% to about 100%, from about 60% to about 80%, or from about 80% to about 100% of the distal portion of the sharp between the tip of the sharp and the proximal portion of the sharp at the skin surface is coated with a therapeutic agent-containing polymer composition. In certain embodiments, about 50%, about 60%, about 80%, or about 100% of the distal portion of the sharp between the tip of the sharp and the proximal portion of the sharp at the skin surface is coated with a therapeutic agent-containing polymer composition.

In certain embodiments, at least about 50% to about 100% of the insertable portion of the sharp is coated with a therapeutic agent-containing polymer composition. In certain embodiments, from about 50% to about 100%, from about 50% to about 80%, from about 50% to about 60%, from about 60% to about 100%, from about 60% to about 80%, or from about 80% to about 100% of the insertable portion of the sharp is coated with a therapeutic agent-containing polymer composition. In certain embodiments, about 50%, about 60%, about 80%, or about 100% of the insertable portion of the sharp is coated with a therapeutic agent-containing polymer composition. In certain embodiments, the insertable portion of sharp is the portion of the sharp that is inserted into a subject for implanting the analyte sensor.

In certain embodiments, at least about 50% to about 100% of the insertable portion of the sharp is coated with a therapeutic agent-containing polymer composition.

In certain embodiments, the therapeutic agent-containing polymer composition on a sharp has a thickness from about 5 μm to about 40 μm. In certain embodiments, the therapeutic agent-containing polymer composition on a sharp has a thickness from about 5 μm to about 40 μm, from about 5 μm to about 30 μm, from about 5 μm to about 20 μm, from about 5 μm to about 10 μm, from about 10 μm to about 40 μm, from about 10 μm to about 30 μm, from about 10 μm to about 20 μm, from about 20 μm to about 40 μm, from about 20 μm to about 30 μm, or from about 30 μm to about 40 μm. In certain embodiments, the therapeutic agent-containing polymer composition has a thickness of about 5 μm, about 10 μm, about 20 μm, about 30 μm, or about 40 μm.

In certain embodiments, the therapeutic agent-containing polymer composition oats from about 20% to about 100% of the circumference of the sharp. In certain embodiments, the therapeutic agent-containing polymer composition coats from about 20% to about 100%, from about 20% to about 80%, from about 20% to about 60%, from about 20% to about 40%, from about 40% to about 100%, from about 40% to about 80%, from about 40% to about 60%, from about 60% to about 100%, from about 60% to about 80%, or from about 80% to about 100% of the circumference of the sharp. In certain embodiments, the therapeutic agent-containing polymer composition coats about 20%, about 30%, about 40%, about 50%, about 60%, about 70%, about 80%, about 90%, or about 100% of the circumference of the sharp.

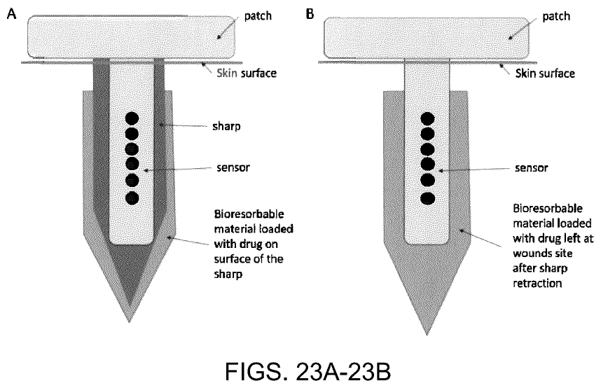

In certain embodiments, the present disclosure comprises a sharp comprising an insertion tip configured to penetrate skin and coated with a therapeutic agent-containing polymer composition, e.g., as shown in FIGS. 23A and 24A. In certain embodiments, suitable sharps can be solid or hollow, beveled or non-beveled and/or circular or non-circular in cross-section. In certain embodiments, the sharp is circular in cross-section. In certain embodiments, the sharp can be comparable in cross-sectional diameter and/or tip design to an acupuncture needle, which can have a cross-sectional diameter of about 250 μm. However, suitable needles can have a larger or smaller cross-sectional diameter if needed for certain particular applications. In certain embodiments, a sharp of the present disclosure has a cross-sectional diameter from about 100 μm to about 500 μm.

In certain embodiments, the sharp can be made of a material such as, but not limited to, a metal, a plastic, a biocompatible material or a combination thereof. In certain embodiments, the sharp can be made of a metal. In certain embodiments, the sharp can be made of stainless steel. In certain embodiments, the therapeutic agent-containing polymer composition is placed directly on the sharp material, e.g., the metal, e.g., the stainless steel, of the sharp.

In certain embodiments, the sharp can include a channel that includes an analyte sensor retained within the channel. Non-limiting examples of analyte sensors that can be included in an analyte sensor of the present disclosure are described in Section III. In certain embodiments, the analyte sensor provided in the sharp, and delivered by the disclosed methods, can be any analyte sensor disclosed herein, e.g., an analyte sensor that includes that includes a therapeutic agent. For example, but not by way of limitation, an analyte sensor can include (i) an in vivo portion comprising at least a first working electrode on a substrate 212, (ii) an active area disposed upon a surface of the first working electrode for detecting an analyte, a membrane permeable to the analyte that coats at least the active area and a counter/reference electrode (e.g., on the substrate). In certain embodiments, the analyte sensor includes a therapeutic agent-containing polymer composition. In certain embodiments, the therapeutic agent provided in the coating of the sharp can be different from the therapeutic agent incorporated into the analyte sensor. Alternatively, the therapeutic agent provided in the coating of the sharp can be the same as the therapeutic agent incorporated into the analyte sensor. For example, but not by way of limitation, the therapeutic agent provided by the coating on the sharp and the therapeutic agent incorporated into the analyte sensor can both bean anti-inflammatory, e.g., dexamethasone.

In certain embodiments, a sharp of the present disclosure can be pre-loaded for packaging and/or shipping. For example, but not by way of limitation, a sharp of the present disclosure can be pre-loaded with an analyte sensor for packaging and/or shipping.

In certain embodiments, the present disclosure provides an insertion device that includes a sharp and an analyte sensor. In certain embodiments, an insertion device of the present disclosure can include:
(a) an insertion tip (e.g., an insertion tip of a sharp) configured to penetrate skin and coated with a therapeutic agent-containing polymer composition; and
(b) an analyte sensor comprising:
   (i) a working electrode;
   (ii) an active area disposed upon the working electrode; and
   (iii) a membrane overcoating at least the active area, wherein the therapeutic agent is separated from the sensor.

In certain embodiments, an insertion device of the present disclosure can include sharp and an analyte sensor wherein the analyte sensor comprises:
an in vivo portion configured to reside below a skin surface of a subject and in contact with interstitial fluid of the subject comprising:
   (a) a working electrode;
   (b) an active area disposed upon the working electrode; and
   (c) a membrane overcoating at least the active area,
wherein at least a portion of the sharp is coated with a therapeutic agent-containing polymer composition.

In certain embodiments, the sharp can be used in a method to deliver a therapeutic agent-containing polymer composition near the analyte sensor in vivo. For example, but not by way of limitation, the method can include providing a sharp coated with a therapeutic agent-containing polymer composition that includes an analyte sensor that is positioned within a channel of the sharp. In certain embodiments, the method can further include penetrating a tissue of a subject with the sharp and inserting the therapeutic agent-containing polymer composition and analyte sensor into the tissue of the subject. In certain embodiments, the method includes retracting the sharp from the tissue of the subject to retain the therapeutic agent-containing polymer composition and analyte sensor in the tissue of the subject.

In certain embodiments, the sharp is retractable. In certain embodiments, when the sharp is retracted back to the applicator, the therapeutic agent-containing polymer composition comprising a therapeutic agent is retained in the tissue in the vicinity of the insertion site of the sharp as shown in FIGS. 23B and 24B. In certain embodiments, the sharp is retracted back into the applicator with a twisting motion which separates the therapeutic agent-containing polymer composition from the sharp and allows the therapeutic agent-containing polymer composition to be retained in the tissue of the subject. In certain embodiments, the sharp is a tear away sharp which contains one or more perforations along a perforation line disposed along a length of the sharp and where a twisting motion allows the sharp to break along the perforations and allows the therapeutic agent-containing polymer composition to separate from the sharp and be retained in the tissue of the subject.

In certain embodiments, the therapeutic agent-containing polymer composition is configured to release the therapeutic agent to an area surrounding an insertion site of the sensor. In certain embodiments, from about 50% to about 100% of the therapeutic agent-containing polymer composition is retained in the tissue after removal of the sharp. In certain embodiments, from about 50% to about 100%, from about 50% to about 80%, from about 50% to about 60%, from about 60% to about 100%, from about 60% to about 80%, or from about 80% to about 100% of the therapeutic agent-containing polymer composition is retained in the tissue after removal of the sharp. In certain embodiments, about 50%, about 60%, about 80%, or about 100% of the therapeutic agent-containing polymer composition is retained in the tissue after removal of the sharp.

In certain embodiments, the present disclosure provides a method of inhibiting an immune response at an analyte sensor insertion site, the method comprising:
(a) introducing into a tissue at the insertion site, a therapeutic agent-containing polymer composition comprising an amount of therapeutic agent suitable for inhibiting an immune response at the insertion site using a sharp, wherein the therapeutic agent-containing polymer composition coats the sharp (e.g., coats the insertion tip of the sharp); and
(b) maintaining the therapeutic agent-containing polymer composition in the tissue following retraction of the insertion tip.

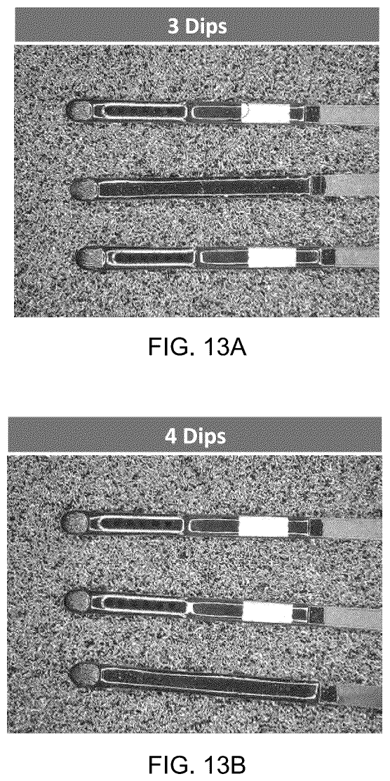

In certain embodiments, the sharp is a part of an introducer as disclosed herein. In certain embodiments, the sharp is a part of a sharp module and/or sensor applicator, e.g., as disclosed in International Publication Nos. WO 2018/136898, WO 2019/236859 and WO 2019/236876 and U.S. Patent Publication No. 2020/0196919, each of which is incorporated by reference in its entirety herein. For example, but not by way of limitation, the sharp can be a part of a sensor applicator as shown in FIG. 32B (e.g., the sharp is noted as 3216), FIG. 34B (e.g., the sharp is noted as 3216), FIG. 40B (e.g., the sharp is noted as 3908) and FIG. 113 (e.g., the sharp is noted as 11308) of WO 2019/236859. In certain embodiments, the sharp can be a part of a sensor module as shown in FIG. 13 of WO 2019/236876 (e.g., the sharp (1318) is incorporated into a sensor module (noted as 1314) for insertion of a sensor (1316)).

Further details regarding non-limiting embodiments of applicators, their components and variants thereof, are described in U.S. Patent Publication Nos. 2013/0150691, 2016/0331283 and 2018/0235520, all of which are incorporated by reference herein in their entireties and for all purposes. In certain embodiments, the sharp is part of a sensor applicator as shown in FIG. 11A of U.S. 2013/0150691 (e.g., the sharp is shown as 1030 and the sensor supported within the sharp is noted as 1102). Further details regarding non-limiting embodiments of sharp modules, sharps, their components and variants thereof, are described in U.S. Patent Publication No. 2014/0171771, which is incorporated by reference herein in its entirety and for all purposes.

The present disclosure further provides methods for coating a sharp described herein with a therapeutic agent-containing polymer composition. In certain embodiments, a polymer solution (e.g., a second polymer solution as described herein, e.g., in Section VI) is applied to the sharp (e.g., the insertion tip of the sharp) to produce a therapeutic agent-containing polymer composition on the sharp. For example, but not by way of limitation, the amount of the polymer included in the polymer solution can be in a range of about 1 mg/mL to about 200 mg/mL, for example, about 1 mg/mL to about 180 mg/mL, about 1 mg/mL to about 160 mg/mL, about 1 mg/mL to about 140 mg/mL, about 1 mg/mL to about 120 mg/mL, about 1 mg/mL to about 100 mg/mL, about 1 mg/mL to about 90 mg/mL, about 1 mg/mL to about 80 mg/mL, about 1 mg/mL to about 70 mg/mL, about 1 mg/mL to about 60 mg/mL, about 1 mg/mL to about 50 mg/mL, about 5 mg/mL to about 150 mg/mL, about 10 mg/mL to about 150 mg/mL, about 20 mg/mL to about 150 mg/mL, about 30 mg/mL to about 150 mg/mL, about 40 mg/mL to about 150 mg/mL, about 50 mg/mL to about 150 mg/mL, or about 60 mg/mL to about 150 mg/mL, or any values in between. In certain embodiments, an amount of the polymer included in the polymer solution can be in a range of about 1 mg/mL to about 20 mg/mL, about 20 mg/mL to about 40 mg/mL, about 40 mg/mL to about 60 mg/mL, about 60 mg/mL to about 80 mg/mL, about 80 mg/mL to about 100 mg/mL, about 90 mg/mL to about 110 mg/mL, about 100 mg/mL to about 120 mg/mL, or about 120 mg/mL to about 140 mg/mL.

In certain embodiments, the polymer solution can further include a solvent selected from water, methanol, ethanol, propanol, isopropanol, n-butanol, iso-butanol, tetrahydrofuran, methyl-tetrahydrofuran, cyclopentyl methyl ether, and a combination thereof. In certain embodiments, the solvent is ethanol.

In certain embodiments, the viscosity of the polymer solution can be in a range of about 50 cP (centiPoise) to about 250 cP, for example, about 60 cP to about 250 cP, about 70 cP to about 250 cP, about 80 cP to about 250 cP, about 90 cP to about 250 cP, about 100 cP to about 250 cP, about 100 cP to about 240 cP, about 100 cP to about 230 cP, about 100 cP to about 220 cP, about 100 cP to about 210 cP, or about 100 cP to about 200 cP, or any values in between. In certain embodiments, a viscosity of the first polymer solution can be in a range of about 50 cP to about 120 cP, about 60 cP to about 130 cP, about 70 cP to about 140 cP, about 80 cP to about 150 cP, about 90 cP to about 160 cP, about 100 cP to about 170 cP, about 110 cP to about 180 cP, about 120 cP to about 190 cP, or about 130 cP to about 200 cP. In certain embodiments, the viscosity of the polymer solution can be in a range of about 10 cP to about 150 cP, e.g., about 10 cP to about 110 cP.

In certain embodiments, a method for manufacturing of sharp of the present disclosure include contacting a polymer solution to the sharp to form a therapeutic agent-containing polymer composition on the sharp. In certain embodiments, the polymer solution is applied to the sharp by a screen printing, a rotary printing, a jetting, an aerosol deposition, a spray coating, a dip coating, a drop-casting, a molding, a spin coating or a brush coating process to generate a therapeutic agent-containing polymer composition on the sharp. In certain embodiments, the polymer solution is applied to the sharp by a dip coating process. In certain embodiments, the sharp is contacted with the polymer solution, e.g., by a dipping process, one or more times until the therapeutic agent-containing polymer composition reaches the desired thickness (e.g., a thickness of about 5 μm to about 40 μm). In certain embodiments, the sharp is contacted with the polymer solution, e.g., by a dipping process, two or more times, three or more times, four or more times or five or more times until the therapeutic agent-containing polymer composition reaches the desired thickness (e.g., a thickness of about 5 μm to about 40 μm).

### VIII. Exemplary Embodiments

A. In certain non-limiting embodiments, the presently disclosed subject matter provides an analyte sensor, comprising:
a sensor tail comprising a non-sensing region and an active sensing region,
wherein the non-sensing region surrounds the active sensing region and comprises a drug-loading structure filled with a therapeutic agent-containing polymer composition.
A1. The analyte sensor of A, wherein the drug-loading structure comprises at least one selected from a slot, a hole, a pore, a groove, and a depression.
A2. The analyte sensor of A or A1, wherein the drug-loading structure comprises two slots symmetrically arranged along the active sensing region.
A2.1. The analyte sensor of any of A-A2, wherein the active sensing region a plurality of sensing spots.
A3. The analyte sensor of A or A1, wherein the drug-loading structure comprises a plurality of holes symmetrically arranged around the active sensing region.
A3.1 The analyte sensor of any one of A-A3, wherein the active sensing region a plurality of sensing spots.
A4. The analyte sensor of A1, A2 or A2.1, wherein the slot is in a shape of an oval, a shape of a polygon, or an irregular shape.
A5. The analyte sensor of A1, A3 or A3.1, wherein the hole is in a shape of a circle, a shape of a regular polygon, or a shape of an irregular polygon.
A5.1. The analyte sensor of any one of A-A5, wherein the therapeutic agent-containing polymer composition comprises a polymer and a therapeutic agent.
A5.2. The analyte sensor of A5.1, wherein the polymer is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and a combination thereof.
A6. The analyte sensor of any one of A-A5, wherein the therapeutic agent-containing polymer composition comprises a copolymer and a therapeutic agent.
A7. The analyte sensor of A6, wherein the copolymer is selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.
A8. The analyte sensor of A7, wherein the polyvinylpyridine-based copolymer is a polyvinylpyridine-co-polystyrene polymer or a derivative thereof.
A9. The analyte sensor of A8, wherein the polyvinylpyridine-co-polystyrene polymer can include 1-50 mer % of styrene units.
A9.1. The analyte sensor of A8, wherein the polyvinylpyridine-co-polystyrene polymer can include 1-20 mer % of styrene units.
A10. The analyte sensor of any one of A-A9, wherein the therapeutic agent is at least one selected from group consisting of an antibiotic agent, an antiviral agent, an anti-inflammatory agent, an anti-cancer agent, an antiplatelet agent, an anticoagulant agent, a coagulant agent, an antiglycolytic agent.
A11. The analyte sensor of any one of A-A10, wherein the therapeutic agent is an anti-inflammatory agent.
A12. The analyte sensor of any one of A-A11, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
A13. The analyte sensor of any one of A-A12, wherein the anti-inflammatory agent is dexamethasone, a derivative thereof, or a salt form thereof.
A14. The analyte sensor of any one of A-A13, wherein the therapeutic agent is in a range of 0.01 wt %-50 wt % based on the total weight of the copolymer within the therapeutic agent-containing polymer composition.
A15. The analyte sensor of any one of A-A14, wherein the therapeutic agent is in a range of 0.01 wt %-40 wt % based on the total weight of the copolymer within the therapeutic agent-containing polymer composition.
A16. The analyte sensor of any one of A-A15, wherein the therapeutic agent-containing polymer composition comprises from about 0.01 μg to about 200 μg of the therapeutic agent.
A17. The analyte sensor of any one of A-A16, wherein the therapeutic agent-containing polymer composition comprises from about 0.01 μg to about 20 μg of the therapeutic agent.
A18. The analyte sensor of any one of A-A17, wherein the therapeutic agent-containing polymer composition comprises from about 0.01 μg to about 10 μg of the therapeutic agent.
A19. The analyte sensor of any one of A-A18, wherein the therapeutic agent-containing polymer composition comprises from about 0.01 μg to about 5 μg of the therapeutic agent.
A20. The analyte sensor of any one of A-A19, wherein the analyte sensor is a subcutaneous sensor.
A21. The analyte sensor of any one of A-A20, wherein the analyte sensor is configured to detect glucose.
B. In certain non-limiting embodiments, the presently disclosed subject matter provides a method of preparing an analyte sensor, the method comprising:
(i) providing a sensor tail (i.e., an in vivo portion) comprising a non-sensing region and an active sensing region;
(ii) removing (e.g., cutting) at least a portion of the non-sensing region of the sensor tail to form a drug-loading structure; and
(iii) filling the drug-loading structure with a therapeutic agent-containing polymer composition.
B1. The method of B, wherein the drug-loading structure comprises at least one selected from a slot, a hole, a pore, a groove, and a depression.
B2. The method of B or B1, wherein the drug-loading structure comprises two slots symmetrically arranged along the active sensing region.
B2.1 The method of any of B-B2, wherein the active sensing region a plurality of sensing spots.
B3. The method of B or B1, wherein the drug-loading structure comprises a plurality of holes symmetrically arranged around the active sensing region.
B3.1 The method of any one of B-B3, wherein the active sensing region a plurality of sensing spots.
B4. The method of B1, B2 or B2, wherein the slot is in a shape of an oval, a shape of a polygon, or an irregular shape.
B5. The method of B1, B3 or B3.1, wherein the hole is in a shape of a circle, a shape of a regular polygon, or a shape of an irregular polygon.
B5.1. The method of any one of B-B5, wherein the therapeutic agent-containing polymer composition comprises a polymer and a therapeutic agent.
B5.2. The method of B5.1, wherein the polymer is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a derivative thereof, and a combination thereof.
B6. The method of any one of B-B5, wherein the therapeutic agent-containing polymer composition comprises a copolymer and a therapeutic agent.
B7. The method of B6, wherein the copolymer is selected from the group consisting of a polyvinylpyridine-based copolymer, a polyvinylimidazole-based copolymer, a polyacrylate-based copolymer, a polyurethane-based copolymer, a polyether urethane-based copolymer, a silicone-based copolymer, a derivative thereof, and a combination thereof.
B8. The method of B7, wherein the polyvinylpyridine-based copolymer is a polyvinylpyridine-co-polystyrene polymer or a derivative thereof.
B9. The method of B8, wherein the polyvinylpyridine-co-polystyrene polymer can include 1-50 mer % of styrene units.
B10. The method of any one of B-B9, wherein the therapeutic agent is at least one selected from group consisting of an antibiotic agent, an antiviral agent, an anti-inflammatory agent, an anti-cancer agent, an antiplatelet agent, an anticoagulant agent, a coagulant agent, an antiglycolytic agent.
B11. The method of any one of B-B10, wherein the therapeutic agent is an anti-inflammatory agent.
B12. The method of B11, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
B13. The method of B11 or B12, wherein the anti-inflammatory agent is dexamethasone, a derivative thereof, or a salt form thereof.
B14. The method of any one of B-B13, wherein the therapeutic agent is in a range of 0.01 wt %-50 wt % based on the total weight of the therapeutic agent-containing polymer composition.
B15. The method of any one of B-B14, wherein the therapeutic agent is in a range of 0.01 wt %-40 wt % based on the total weight of the copolymer within the therapeutic agent-containing polymer composition.
B16. The method of any one of B-B15, wherein the therapeutic agent-containing polymer composition comprises from about 0.01 μg to about 200 μg of the therapeutic agent.
B17. The method of any one of B-B16, wherein the therapeutic agent-containing polymer composition comprises from about 0.01 μg to about 20 μg of the therapeutic agent.
B18. The method of any one of B-B17, wherein the therapeutic agent-containing polymer composition comprises from about 0.01 μg to about 10 μg of the therapeutic agent.
B19. The method of any one of B-B18, wherein the therapeutic agent-containing polymer composition comprises from about 0.01 μg to about 5 μg of the therapeutic agent.
B20. The method of any one of B-B18, wherein the active sensing region comprises a plurality of sensing spots.
B21. The method of any one of B-B20, wherein the analyte sensor is a subcutaneous sensor.
B22. The method of any one of B-B21, wherein the analyte sensor is configured to detect glucose.
C. In certain non-limiting embodiments, the presently disclosed subject matter provides a method for coating an analyte sensor,
the analyte sensor comprising a sensor tail comprising a first region not comprising any sensing spot and a second region comprising a plurality of sensing spots,
wherein the method comprises:
preparing a first polymer solution and a second polymer solution differing from the first polymer solution;
dipping the sensor tail in the first polymer solution to coat the first region and the second region comprising the plurality of sensing spots;
   optionally drying the first polymer solution on the sensor tail to form a first polymer membrane;
dipping the sensor tail in the second polymer solution so that the second polymer solution coats the first region of the sensor tail but not the second region comprising the plurality of sensing spots; and
   optionally drying the second polymer solution on the sensor tail to form a second polymer membrane,
wherein the second polymer solution comprises a therapeutic agent.
C1. The method of C, wherein dipping the sensor tail in the first polymer solution is proceeded before dipping the sensor tail in the second polymer solution.
C2. The method of C, wherein dipping the sensor tail in the second polymer solution is proceeded before dipping the sensor tail in the first polymer solution.
C3. The method of C, wherein the second polymer membrane is on top of at least a portion of the first polymer membrane.
C4. The method of C, wherein the second polymer membrane is underneath at least a portion of the first polymer membrane.
C5. The method of C, wherein the drying the first polymer solution on the sensor tail to form the first polymer membrane and drying the second polymer solution on the sensor tail to form the second polymer membrane are proceeded concurrently after the dipping the sensor tail in the first polymer solution and the dipping the sensor tail in the second polymer solution are completed.
C6. The method of any one of C-C5, wherein the first polymer solution comprises a first copolymer selected from the group consisting of a polyvinylpyridine-based copolymer, a polyvinylimidazole-based copolymer, and a combination thereof.
C7. The method of C6, wherein the polyvinylpyridine-based copolymer is a polyvinylpyridine-co-polystyrene polymer.
C8. The method of any one of C-C7, wherein the second polymer solution comprises a second copolymer selected from the group consisting of a polyvinylpyridine-based copolymer, a polyvinylimidazole-based copolymer, and a combination thereof.
C9. The method of C8, wherein the polyvinylpyridine-based copolymer is a polyvinylpyridine-co-polystyrene polymer.
C10. The method of any one of C-C9, wherein a viscosity of the first polymer solution is in a range of about 50 cP to about 250 cP.
C11. The method of any one of C-C10, wherein a viscosity of the second polymer solution is in a range of about 50 cP to about 250 cP.
C11.1. The method of any one of C-C10, wherein a viscosity of the second polymer solution is in a range of about 50 cP to about 250 cP.
C12. The method of any one of C-C11.1, wherein the first polymer membrane is a mass-limiting membrane.
C13. The method of any one of C-C12, wherein the second polymer membrane is a drug-eluting membrane.
C14. The method of any one of C-C13, wherein the therapeutic agent is at least one selected from group consisting of an antibiotic agent, an antiviral agent, an anti-inflammatory agent, an anti-cancer agent, an antiplatelet agent, an anticoagulant agent, a coagulant agent, an antiglycolytic agent.
C15. The method of any one of C-C14, wherein the therapeutic agent is an anti-inflammatory agent.
C16. The method of C15, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
C17. The method of C16, wherein the anti-inflammatory agent is dexamethasone, a derivative thereof, or a salt form thereof.
C18. The method of any one of C-C17, wherein the therapeutic agent is in a range of 0.01 wt %-50 wt % based on a total weight of the second copolymer in the second polymer solution.
C19. The method of any one of C-C18, wherein the therapeutic agent is in a range of 0.01 wt %-40 wt % based on a total weight of the second copolymer in the second polymer solution.
C20. The method of any one of C-C18, wherein the second polymer solution further comprises a solvent.
C21. The method of C20, wherein the solvent comprises ethanol.
D. In certain non-limiting embodiments, the presently disclosed subject matter provides an analyte sensor, comprising:
(i) a sensor tail comprising a first region not comprising any sensing spot and a second region comprising a plurality of sensing spots,
(ii) a first polymer membrane coated on the sensor tail in the first region and the second region comprising the plurality of sensing spots; and
(iii) a second polymer membrane, differing from the first polymer membrane, coated on the sensor tail in the first region, and not in the second region, wherein the second polymer membrane comprising a therapeutic agent.
D1. The analyte sensor of D, wherein the second polymer membrane is on top of the first polymer membrane.
D2. The analyte sensor of D, wherein the second polymer membrane is underneath the first polymer membrane.
D3. The analyte sensor of D or D1, wherein the first polymer membrane is composed of a first polymer comprising a first copolymer selected from the group consisting of a polyvinylpyridine-based copolymer, a polyvinylimidazole-based copolymer, and a combination thereof.
D4. The analyte sensor of D3, wherein the polyvinylpyridine-based copolymer is a polyvinylpyridine-co-polystyrene polymer.
D5. The analyte sensor of any one of D-D4, wherein the second polymer membrane is composed of a second polymer comprising a second copolymer selected from the group consisting of a polyvinylpyridine-based copolymer, a polyvinylimidazole-based copolymer, and a combination thereof.
D6. The analyte sensor of D5, wherein the polyvinylpyridine-based copolymer is a polyvinylpyridine-co-polystyrene polymer.
D7. The analyte sensor of any one of D-D6, wherein the first polymer membrane is a mass-limiting membrane.
D7.1. The analyte sensor of any one of D-D7, wherein the second polymer membrane is a drug-eluting membrane.
D7.2. The analyte sensor of any one of D-D7.1, the therapeutic agent is an anti-inflammatory agent.
D8. The analyte sensor of D7.2, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
D9. The analyte sensor of D7.2 or D8, wherein the anti-inflammatory agent is dexamethasone, a derivative thereof, or a salt form thereof.
D10. The analyte sensor of any one of D-D9, wherein the therapeutic agent is in a range of 0.01 wt %-50 wt % based on a total weight of the second copolymer in the second polymer membrane.
D11. The analyte sensor of any one of D-D10, wherein the therapeutic agent is in a range of 0.01 wt %-40 wt % based on a total weight of the second copolymer in the second polymer membrane.
D12. The method of any one of D-D11, wherein second polymer membrane comprises from about 0.01 μg to about 200 μg of the therapeutic agent.
D13. The method of any one of D-D12, wherein second polymer membrane comprises from about 0.01 μg to about 20 μg of the therapeutic agent.
D14. The method of any one of D-D13, wherein second polymer membrane comprises from about 0.01 μg to about 10 μg of the therapeutic agent.
D15. The method of any one of D-D14, wherein second polymer membrane comprises from about 0.01 μg to about 5 μg of the therapeutic agent.
E. A method for manufacturing an analyte sensor comprising a therapeutic agent, the method comprising:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer;
(iii) preparing a second polymer solution comprising a second polymer and a therapeutic agent;
(iv) dipping the in vivo portion in the first polymer solution to coat the first region and the second region to generate a first polymer membrane; and
(v) dipping the first region in the second polymer solution to coat the first region and not the second region to generate a second polymer membrane comprising the therapeutic agent on top of the first polymer membrane.
E1. A method for manufacturing an analyte sensor comprising a therapeutic agent, the method comprising:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer;
(iii) preparing a second polymer solution comprising a second polymer and a therapeutic agent;
(iv) dipping the first region in the second polymer solution to coat the first region and not the second region to generate a second polymer membrane comprising the therapeutic agent; and
(v) dipping the in vivo portion in the first polymer solution to coat the first region and the second region to generate a first polymer membrane on top of the second polymer membrane.
E2. The method of E or E1, wherein the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.
E3. The method of E2, wherein the therapeutic agent is an anti-inflammatory agent.
E4. The method of E3, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
E5. The method of any one of E2-E4, wherein the anti-inflammatory agent is dexamethasone, a derivative thereof, or a salt form thereof.
E6. The method of any one of E-E5, wherein the second polymer solution comprises the polymer, the therapeutic agent and a solvent.
E7. The method of E6, wherein the solvent comprises an alcohol, a buffer or a combination thereof.
E8. The method of E7, wherein the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof.
E8.1. The method of E7, wherein the alcohol is ethanol.
E9. The method of any one of E-E8, wherein the second polymer solution further comprises a crosslinker.
E10. The method of any one of E-E9, wherein the therapeutic agent is present in the second polymer solution in a range from about 0.01 wt % to about 50 wt % based on the total weight of the second polymer solution.
E11. The method of any one of E-E10, wherein the polymer is present in the second polymer solution in a range from about 0.01 wt % to about 50 wt % based on the total weight of the second polymer solution.
E12. The method of any one of E-E11, wherein the crosslinker is present in the second polymer solution in a range from about 0.01 wt % to about 50 wt % based on the total weight of the second polymer solution.
E13. The method of any one of E-E12, wherein the second polymer solution has a viscosity of about 20 cP to about 250 cP.
E14. The method of any one of E-E12, wherein the second polymer solution has a viscosity of about 10 cP to about 150 cP.
E14.1. The method of any one of E-E13, wherein the second polymer solution has a viscosity of about 20 cP to about 80 cP.
E15. The method of any one of E-E14.1, wherein the second polymer of the second polymer solution is selected from the group consisting of a polyvinylpyridine-based polymer, a polyvinylimidazole-based polymer, a poly acrylate-based polymer, a polyurethane-based polymer, a polyether urethane-based polymer, a silicone-based polymer, a copolymer thereof, a derivative thereof, and a combination thereof.
E16. The method of E15, wherein the polyvinylpyridine-based polymer is a polyvinylpyridine-based copolymer.
E17. The method of E16, wherein the polyvinylpyridine-based copolymer is a polyvinylpyridine-co-polystyrene polymer.
E17.1. The method of E15, wherein the polymer is a polyurethane-based polymer.
E18. The method of any one of E-E17.1, wherein the first region is dipped in the second polymer solution for a time period of about 1 second to about 5 seconds.
E19. The method of any one of E-E18, further comprising drying the second polymer solution for a time period of about 1 to about 20 minutes after dipping.
E20. The method of any one of E-E19, wherein the first region of the analyte sensor is dipped into the second polymer solution one or more times to generate a polymer membrane having a thickness between about 5 μm to about 40 μm.
E21. The method of any one of E-E20, wherein the first region of the analyte sensor is dipped into the second polymer solution at least three times.
E22. The method of any one of E-E21, wherein the first region is ablated or planed prior to the dipping of the first region into the second polymer solution.
E23. The method of any one of E-E22, wherein the second region has a length of about 5 μm to about 50 μm.
E24. The method of any one of E-E23, wherein the first region has a length of about 5 μm to about 50 μm.
E25. The method of any one of E-E24, wherein the first region has a length of about 5 μm to about 20 μm.
E26. The method of any one of E-E25, wherein the active area comprises a plurality of sensing regions.
E27. The method of E26, wherein at least one of the plurality of sensing regions is a spot.
E28. The method of any one of E-E27, wherein the first polymer of the first polymer solution is different from the second polymer.
E29. The method of any one of E-E28, wherein the first polymer of the first polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.
E30. The method of E29, wherein the polyvinylpyridine-based polymer is a polyvinylpyridine-based copolymer.
E31. The method of E30, wherein the polyvinylpyridine-based copolymer is a polyvinylpyridine-co-polystyrene polymer.
E32. The method of any one of E-E31, wherein the in vivo portion of the analyte sensor is dipped in the first polymer solution for a time period of about 1 second to about 5 seconds.
E33. The method of any one of E-E32, further comprising drying the first polymer solution for a period of about 1 to about 20 minutes after dipping.
E34. The method of any one of E-E33, wherein the in vivo portion of the analyte sensor is dipped into the first polymer solution one or more times to generate a first polymer membrane having a thickness between about 5 μm to about 40 μm.
E35. The method of any one of E-E34, wherein the in vivo portion of the analyte sensor is dipped into the first polymer solution at least five times.
E36. The method of any one of E-E35, wherein the ratio of the thinnest point to the thickest point of the second polymer membrane is less than about 0.9.
F. A method for manufacturing an analyte sensor comprising a therapeutic agent, the method comprising:
(i) providing an analyte sensor comprising an in vivo portion configured to reside below a skin surface of a subject and in contact with interstitial fluid of the subject, wherein the in vivo portion comprises a second region comprising an active area and a first region distal to the second region;
(ii) preparing a first polymer solution comprising a first polymer or providing a first polymer solution comprising a first polymer;
(iii) preparing a second polymer solution comprising a second polymer and a therapeutic agent or providing a second polymer solution comprising a second polymer and a therapeutic agent;
(iv) contacting the in vivo portion with the first polymer solution to coat the first region and the second region to generate a first polymer membrane; and
(v) contacting the first region with the second polymer solution to coat the first region but not the second region to generate a second polymer membrane comprising the therapeutic agent.
F1. The method of F, wherein contacting the in vivo portion with the first polymer solution comprises a screen printing, a rotary printing, a jetting, an aerosol deposition, a spray coating, a dip coating, a drop-casting, a spin coating or a brush coating process.
F2. The method of F1, wherein contacting the first region with the second polymer solution comprises a screen printing, a rotary printing, a jetting, an aerosol deposition, a spray coating, a dip coating, a drop-casting, a spin coating or a brush coating process.
F3. The method of any one of F-F2, wherein the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.
F4. The method of F3, wherein the therapeutic agent is an anti-inflammatory agent.
F5. The method of F4, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
F6. The method of any one of F3-F5, wherein the anti-inflammatory agent is dexamethasone, a derivative thereof, or a salt form thereof.
F7. The method of any one of F-F6, wherein the second polymer solution comprises the polymer, the therapeutic agent and a solvent.
F8. The method of F7, wherein the solvent comprises an alcohol, a buffer or a combination thereof.
F9. The method of F8, wherein the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof.
F9.1 The method of F9, wherein the solvent is ethanol.
F10. The method of any one of F-F9.1, wherein the second polymer solution further comprises a crosslinker.
F11. The method of any one of F-F10, wherein the therapeutic agent is present in the second polymer solution in a range from about 0.01 wt % to about 50 wt % based on the total weight of the polymer solution.
F12. The method of any one of F-F11, wherein the polymer is present in the second polymer solution in a range from about 0.01 wt % to about 50 wt % based on the total weight of the polymer solution.
F13. The method of any one of cF2-F12, wherein the crosslinker is present in the second polymer solution in a range from about 0.01 wt % to about 50 wt % based on the total weight of the polymer solution.
F14. The method of any one of F-F13, wherein the second polymer solution has a viscosity of about 20 cP to about 250 cP.
F14.1. The method of any one of F-F13, wherein the second polymer solution has a viscosity of about 1 cP to about 150 cP.
F15. The method of F14.1, wherein the second polymer solution has a viscosity of about 20 cP to about 80 cP.
F16. The method of any one of F-F15, wherein the second polymer of the second polymer solution comprises is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.
F17. The method of F16, wherein the polyvinylpyridine-based polymer is a polyvinylpyridine-based copolymer.
F18. The method of F17, wherein the polyvinylpyridine-based copolymer is a polyvinylpyridine-co-polystyrene polymer.
F19. The method of any one of F-F18, wherein the first region is contacted with the second polymer solution for a time period of about 1 second to about 5 seconds.
F20. The method of any one of F-F19 further comprising drying the second polymer solution for a period of about 1 to about 20 minutes after contact.
F21. The method of any one of F-F20, wherein the first region of the analyte sensor is contacted with the second polymer solution one or more times to generate a second polymer membrane having a thickness between about 5 μm to about 40 μm.
F22. The method of any one of F-F21, wherein the first region of the analyte sensor is contacted with the second polymer solution at least three times.
F23. The method of any one of F-F22, wherein the second region is ablated or planed prior to the contact of the second region with the second polymer solution.
F24. The method of any one of F-F23, wherein the second region has a length of about 5 μm to about 50 μm.
F25. The method of any one of F-F24, wherein the first region has a length of about 5 μm to about 50 μm.
F26. The method of any one of F-F25, wherein the first region has a length of about 5 μm to about 20 μm.
F27. The method of any one of F-F26, wherein the active area comprises a plurality of sensing regions.
F28. The method of any one of F-F27, wherein at least one of the plurality of sensing regions is a spot.
F29. The method of any one of F-F28, wherein contacting the in vivo portion with the first solution is performed before contacting the first region with the second polymer solution.
F30. The method of F29, wherein the second polymer membrane is at least partially covering the first polymer membrane.
F31. The method of any one of F-F28, wherein contacting the first region with the second polymer solution is performed before contacting the in vivo portion with the first solution.
F32. The method of F31, wherein the first polymer membrane is at least partially covering the second polymer membrane.
F33. The method of any one of F-F32, wherein the ratio of the thinnest point to the thickest point of the second polymer membrane is less than about 0.9.
F34. The method of any one of E-F33, wherein the total amount of the therapeutic agent in the second polymer membrane is from about 0.01 μg to about 500 μg.
F35. The method of any one of E-F34, wherein the total amount of the therapeutic agent in the second polymer membrane is from about 0.01 μg to about 100 μg.
F36. The method of any one of E-F35, wherein the total amount of the therapeutic agent in the second polymer membrane is from about 0.01 μg to about 10 μg.
F37. The method of any one of E-F36, wherein the total amount of the therapeutic agent in the second polymer membrane is less than about 5 μg.
G. A method for manufacturing an analyte sensor comprising a therapeutic agent, the method comprising:
(a) providing an analyte sensor comprising (i) a working electrode, (ii) a counter and/or a reference electrode and (iii) an active area;
(b) preparing a first polymer solution comprising a first polymer or providing a first polymer solution comprising a first polymer;
(c) preparing a second polymer solution comprising a second polymer and a therapeutic agent or providing a second polymer solution comprising a second polymer and a therapeutic agent;
(d) depositing the second polymer solution on at least a portion of the counter and/or a reference electrode to generate a therapeutic agent-containing polymer composition; and
(e) depositing the first polymer solution on the in vivo portion to generate a first polymer membrane on top at least a portion of the therapeutic agent-containing polymer composition.
G1. The method of G, wherein depositing the second polymer solution on at least a portion of the counter and/or reference electrode comprises a screen printing, a rotary printing, a jetting, an aerosol deposition, a spray coating, a dip coating, a drop-casting, a spin coating or a brush coating process.
G2. The method of G or G1, wherein depositing the first polymer solution on the in vivo portion comprises a screen printing, a rotary printing, a jetting, an aerosol deposition, a spray coating, a dip coating, a drop-casting, a spin coating or a brush coating process.
G3. The method of any one of G-G2, wherein depositing the second polymer solution on the counter and/or reference electrode comprising depositing the second polymer solution onto the counter and/or reference electrode as two or more spots of the therapeutic agent-containing polymer composition.
G4. The method of G3, wherein at least one of the spots has a circle shape, an oval shape, a regular polygon shape or an irregular polygon shape.
G5. The method of G3 or G4, wherein at least two of the two or more spots comprise the same amount of the therapeutic agent.
G6. The method of G3 or G4, wherein the at least two of the two or more spots comprise different amounts of the therapeutic agent.
G7. The method of G6, wherein the difference in the amount of therapeutic agent between the at least two of the two or more spots is about 10% or more.
G8. The method of any one of G3-G7, wherein at least two of the two or more spots comprise the same thickness.
G9. The method of any one of G3-G7, wherein at least two of the two or more spots comprise different thicknesses.
G10. The method of G9, wherein the difference in the thickness of two spots is about 10% or more.
H. A method for fabricating an analyte sensor comprising a therapeutic agent, the method comprising.
(i) patterning a plurality of first conductive layers on a substrate to generate a plurality of working electrodes;
(ii) patterning a plurality of second conductive layers on the substrate to generate a plurality of counter and/or reference electrodes;
(iii) forming one or more spots of a therapeutic agent-containing polymer composition on each counter and/or reference electrode of the plurality of counter and/or reference electrodes, wherein the therapeutic agent-containing polymer composition comprises a polymer and a therapeutic agent; and
(iv) singulating individual analyte sensors from the substrate, wherein each individual analyte sensor comprises at least one working electrode and at least one counter and/or reference electrode.
H1. The method of H, wherein the one or more spots has a circle shape, an oval shape, a regular polygon shape or an irregular polygon shape.
H2. The method of H or H1, wherein two or more spots are formed on the counter and/or reference electrode, wherein at least two of the two or more spots comprise the same amount of the therapeutic agent.
H3. The method of H or H1, wherein two or more spots are formed on the counter and/or reference electrode, wherein at least two of the two spots comprise different amounts of the therapeutic agent.
H4. The method of H3, wherein the difference in the amount of therapeutic agent between the two or more spots is about 10% or more.
H5. The method of any one of H-H4, wherein two or more spots are formed on the counter and/or reference electrode, wherein at least two of the two spots comprise the same thickness.
H6. The method of any one of H-H4, wherein two or more spots are formed on the counter and/or reference electrode, wherein at least two of the two spots comprise different thicknesses.
H7. The method of H6, wherein the difference in the thickness of the two or more discrete area is about 10% or more.
H8. The method of any one of G-H7, wherein forming the one or more spots of a therapeutic agent-containing polymer composition on each of the plurality of counter and/or reference electrodes comprising dispensing two or more layers of a polymer solution comprising the therapeutic agent.
H9. The method of H8, wherein at least one of the two or more spots of a therapeutic agent-containing polymer composition is formed by dispensing two layers of the polymer solution and a second spot of the two or more spots of a therapeutic agent-containing polymer composition is formed by dispensing three layers of the polymer solution.
H10. The method of any one of G-H9, wherein the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.
H11. The method of H10, wherein the therapeutic agent is an anti-inflammatory agent.
H12. The method of H11, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
H13. The method of any one of H10-H12, wherein the anti-inflammatory agent is dexamethasone, a derivative thereof, or a salt form thereof.
H14. The method of any one of G-G10 and H8-H13, wherein the polymer solution comprises the polymer, the therapeutic agent and a solvent.
H15. The method of H14, wherein the solvent comprises an alcohol, a buffer or a combination thereof.
H16. The method of H15, wherein the alcohol is selected from the group consisting of methanol, ethanol, 1-propanol, 2-propanol (isopropanol), 2,2-dimethyl-1-propanol, 1-butanol, 2-butanol, isobutanol, 3-methyl-1-butanol, 3-methyl-2-butanol, 2-methyl-1-propanol, 2-methyl-2-propanol, 1-pentanol, 2-pentanol, 2-methyl-1-pentanol, cyclopentanol, 1-hexanol, cyclohexanol, 1-heptanol, 1-octanol, 1-nonanol, 2-propen-1-ol, phenylmethanol, diphenylmethanol, triphenylmethanol and a combination thereof.
H16.1. The method of H16, wherein the solvent is ethanol.
H17. The method of any one of G-G10 and H8-H16, wherein the polymer solution further comprises a crosslinker.
H18. The method of any one of G-G10 and H8-H17, wherein the therapeutic agent is present in the polymer solution in a range from about 0.01 wt % to about 50 wt % based on the total weight of the polymer solution.
H19. The method of any one of G-G10 and H8-H18, wherein the polymer is present in the polymer solution in a range from about 0.01 wt % to about 50 wt % based on the total weight of the polymer solution.
H20. The method of any one of G-G10 and H8-H19, wherein the crosslinker is present in the polymer solution in a range from about 0.01 wt % to about 50 wt % based on the total weight of the polymer solution.
H21. The method of any one of G-G10 and H8-H20, wherein the polymer solution has a viscosity of about 20 cP to about 250 cP.
H22. The method of any one of G-G10 and H8-H21, wherein the polymer solution has a viscosity of about 10 cP to about 150 cP.
H22.1. The method of any one of G-G10 and H8-H22, wherein the polymer solution has a viscosity of about 20 cP to about 80 cP.
H23. The method of any one of G-G10 and H8-H22, wherein the polymer of the polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.
H24. The method of H23, wherein the polyvinylpyridine-based polymer is a polyvinylpyridine-based copolymer.
H25. The method of H24, wherein the polyvinylpyridine-based copolymer is a polyvinylpyridine-co-polystyrene polymer.
H25.1. The method of H23, wherein the polymer is a polyurethane-based polymer.
H26. The method of any one of H-H25.1 further comprising forming an active area on each of the plurality of working electrodes.
H27. The method of any one of G-G9 and H26, wherein the active area comprises a plurality of sensing regions.
H28. The method of H27, wherein at least one of the plurality of sensing regions is a sensing spot.
H29. The method of any one of H8-H28 further comprising forming a membrane over at least a portion of the therapeutic agent-containing polymer composition of the individual analyte sensors.
H30. The method of H29, wherein the membrane comprises a polymer selected from a group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.
I. The present disclosure provides an insertion device comprising:
a) an insertion tip (e.g., an insertion tip of a sharp) configured to penetrate skin and coated with a therapeutic agent-containing polymer composition; and
b) a sensor comprising:
   i. a working electrode;
   ii. an active area disposed upon the working electrode; and
   iii. a membrane overcoating at least the active area, wherein the therapeutic agent is separated from the sensor.
I1. The insertion device of I, wherein the insertion tip is retractable.
I2. The insertion device of I or I1, wherein the therapeutic agent-containing polymer composition overcoating the insertion tip is detachably attached to the insertion tip.
I3. The insertion device of any one of I-I2, wherein the therapeutic agent-containing polymer composition is configured to remain in a tissue of a subject upon retraction of the insertion tip.
I4. The insertion device of any one of I-I3, wherein the therapeutic agent-containing polymer composition is configured to release the active agent to an area surrounding an insertion site of the sensor.
I5. The insertion device of any one of I-I4, wherein the therapeutic agent is selected from the group consisting of an antibiotic agent, an antiviral agent, an anti-inflammatory agent, an anti-cancer agent, an antiplatelet agent, an anticoagulant agent, an antiglycolytic agent, and combinations thereof.
I6. The insertion device of any one of I-I5, wherein the therapeutic agent is an anti-inflammatory agent.
I7. The insertion device of I6, wherein the anti-inflammatory agent is selected from the group consisting of triamcilolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and combinations thereof.
I8. The insertion device of I6 or I7, wherein the anti-inflammatory agent is dexamethasone.
I9. The insertion device of any one of I-I8, wherein the polymer is a bioresorbable polymer.
I10. The insertion device of any one of I-I9, wherein the polymer is selected from the group consisting of a polyvinylpyridine-based polymer, a polyvinylimidazole-based polymer, a polyacrylate-based polymer, a polyurethane-based copolymer, a polyether urethane-based polymer, a silicone-based polymer, a poly caprolactone polymer, a polylactide polymer, a polyglycolide polymer, a polyethylene glycol polymer, a derivative thereof, and combinations thereof.
I11. The insertion device of I9 or I10, wherein the therapeutic agent is covalently bound to the polymer.
I12. The insertion device of I9 or I10, wherein the therapeutic agent is not bound to the polymer.
I13. The insertion device of any one of I-I12, wherein the membrane comprises poly(4-vinylpyridine).
I14. The insertion device of any one of I-I13, wherein the active area comprises one or more enzymes configured to detect an analyte.
I15. The insertion device of I14, wherein the analyte is selected from the group consisting of glutamate, glucose, ketones, lactate, oxygen, glycated hemoglobin (HbA1c), albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, and combinations thereof.
I16. The insertion device of any one of I-I15, wherein the active area further comprises an electron transfer agent.
I17. The insertion device of I16, wherein the electron transfer agent comprises a transition metal complex.
I18. The insertion device of any one of I-I17, wherein the sensor further comprises an albumin and optionally a pH buffer.
I19. The insertion device of any one of I-I18, wherein the sensor further comprises a reference electrode, a counter electrode, or both a reference electrode and a counter electrode.
I20. The insertion device of any one of I-I19, wherein the sensor further comprises a second active area that is responsive to a second analyte.
I21. The insertion device of I20, wherein the second analyte is glucose, lactate, or ketone.
I22. The insertion device of any one of I-I21, wherein the therapeutic agent-containing polymer composition is configured to release the therapeutic agent at a predetermined release rate.
I23. The insertion device of any one of I-I22, wherein the therapeutic agent-containing polymer composition can continuously release the therapeutic agent at a drug delivery rate of about 0.01 μg/day to about 1 mg/day of the therapeutic agent.
I24. The insertion device of any one of I-I23, wherein the therapeutical agent is in a range of 0.01 wt %-50 wt % based on a total weight of the therapeutic agent-containing polymer composition and/or wherein the therapeutic agent-containing polymer composition about 0.01 μg to about 100 μg of the therapeutic agent.
I25. The insertion device of any one of I-I24, wherein the therapeutic agent-containing polymer composition further comprises a crosslinker.
J. A method of inhibiting an immune response at an analyte sensor insertion site, the method comprising:
a) introducing into a tissue at the insertion site by use of a sharp, a therapeutic agent-containing polymer composition comprising an effective amount of a therapeutic agent suitable for inhibiting an immune response at the insertion site, wherein the therapeutic agent-containing polymer composition coats a portion of the sharp (e.g., the insertion tip of the sharp); and
b) maintaining the therapeutic agent-containing polymer composition in the tissue following retraction of the insertion tip.
J.1 A method of inserting an analyte sensor insertion site, the method comprising:
a) introducing into a tissue at the insertion site by use of a sharp, a therapeutic agent-containing polymer composition comprising an effective amount of a therapeutic agent suitable for inhibiting an immune response at the insertion site, wherein the therapeutic agent-containing polymer composition coats a portion of the sharp (e.g., the insertion tip of the sharp); and
b) maintaining the therapeutic agent-containing polymer composition in the tissue following retraction of the insertion tip.
J1. The method of J or J.1, wherein therapeutic agent-containing polymer composition overcoating the insertion tip is detachably attached to the insertion tip.
J2. The method of J1 or J2, wherein the therapeutic agent is selected from the group consisting of an antibiotic agent, an antiviral agent, an anti-inflammatory agent, an anti-cancer agent, an antiplatelet agent, an anticoagulant agent, an antiglycolytic agent, and combinations thereof.
J3. The method of any one of J-J2, wherein the therapeutic agent is an anti-inflammatory agent.
J4. The method of J3, wherein the anti-inflammatory agent is selected from the group consisting of triamcilolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, and combinations thereof.
J5. The method of J3 or J4, wherein the anti-inflammatory agent is dexamethasone.
J6. The method of any one of J-J5, wherein the polymer is a bioresorbable polymer.
J7. The method of any one of J-J6, wherein the polymer is selected from the group consisting of a polyvinylpyridine-based polymer, a polyvinylimidazole-based polymer, a poly acrylate-based polymer, a polyurethane-based copolymer, a polyether urethane-based polymer, a silicone-based polymer, a polycaprolactone polymer, a polylactide polymer, a polyglycolide polymer, a polyethylene glycol polymer, a derivative thereof, and combinations thereof.
J8. The method of J6 or J7, wherein the therapeutic agent is covalently bound to the polymer.
J9. The method of J6 or J7, wherein the therapeutic agent is not bound to the polymer.
J10. The method of any one of J-J9, wherein the therapeutic agent-containing polymer composition is configured to release the therapeutic agent at a predetermined release rate.
J11. The method of any one of J-J10, further comprising introducing into the tissue an analyte sensor comprising a working electrode, an active area disposed on the working electrode, and a membrane overcoating at least the active area, wherein the analyte sensor is separated (e.g., at a distance) from the therapeutic agent.
J12. The method of J11, wherein the membrane comprises poly(4-vinylpyridine).
J13. The method of J11 or J12, wherein the active area comprises one or more enzymes configured to detect an analyte.
J14. The method of J13, wherein the analyte is selected from the group consisting of glutamate, glucose, ketones, lactate, oxygen, glycated hemoglobin (HbA1c), albumin, alcohol, alkaline phosphatase, alanine transaminase, aspartate aminotransferase, bilirubin, blood urea nitrogen, calcium, carbon dioxide, chloride, creatinine, hematocrit, aspartate, asparagine, magnesium, oxygen, pH, phosphorus, potassium, sodium, total protein, uric acid, and combinations thereof.
J15. The method of J13 or J14, wherein the analyte is glucose, lactate, or ketone.
J15. The method of any one of J1-J15, wherein the active area further comprises an electron transfer agent.
J16. The method of J15, wherein the electron transfer agent comprises a transition metal complex.
J17. The method of any one of J11-J16, wherein the analyte sensor further comprises an albumin and optionally a pH buffer.
J18. The method of any one of J11-J17, wherein the analyte sensor further comprises a reference electrode, a counter electrode, or both a reference electrode and a counter electrode.
J19. The method of any one of J1-J18, wherein the therapeutic agent-containing polymer composition is configured to release the active agent to an area surrounding an insertion site of the sensor.
J20. The method of any one of J1-J19, wherein the therapeutic agent-containing polymer composition can continuously release the therapeutic agent at a drug delivery rate of about 0.01 μg/day to about 1 mg/day of the therapeutic agent.
J21. The method of any one of J1-J20, wherein the therapeutical agent is in a range of 0.01 wt %-50 wt % based on a total weight of the therapeutic agent-containing polymer composition and/or wherein the therapeutic agent-containing polymer composition about 0.01 μg to about 100 μg of the therapeutic agent.
J22. The method of any one of J1-J21, wherein the therapeutic agent-containing polymer composition further comprises a crosslinker.
K. In certain non-limiting embodiments, the presently disclosed subject matter provides an analyte sensor, comprising:
(a) an in vivo portion configured to reside below a skin surface of a subject and in contact with interstitial fluid of the subject, wherein the in vivo portion comprises:
   (i) at least a first working electrode on a substrate;
   (ii) an active area disposed upon a surface of the first working electrode for detecting an analyte;
   (iii) a mass transport limiting membrane permeable to the analyte that coats at least the active area;
   (iv) a counter/reference electrode on the substrate; and
(b) at least two or more spots of a therapeutic agent-containing polymer composition on a structure of the in vivo portion.
K1. The analyte sensor of K, wherein the structure of the in vivo portion is the counter/reference electrode.
K2. The analyte sensor of K or K1, wherein the at least one of the two or more spots has a circle shape, an oval shape, a regular polygon shape or an irregular polygon shape.
K3. The analyte sensor of any one of K-K2, wherein two or more spots are formed on the counter and/or reference electrode, wherein at least two of the two or more spots comprise the same amount of the therapeutic agent.
K4. The analyte sensor of any one of K-K2, wherein two or more spots are formed on the counter and/or reference electrode, wherein at least two of the two spots comprise different amounts of the therapeutic agent.
K5. The analyte sensor of K4, wherein the difference in the amount of therapeutic agent between the two or more spots is about 10% or more.
K6. The analyte sensor of any one of K-K5, wherein two or more spots are formed on the counter and/or reference electrode, wherein at least two of the two spots comprise the same thickness.
K7. The analyte sensor of any one of K-K5, wherein two or more spots are formed on the counter and/or reference electrode, wherein at least two of the two spots comprise different thicknesses.
K8. The analyte sensor of K7, wherein the difference in the thickness of the two or more discrete area is about 10% or more.
K9. The analyte sensor of any one of K-K8, wherein forming the two or more spots of a therapeutic agent-containing polymer composition on each of the plurality of counter and/or reference electrodes comprising dispensing two or more layers of a polymer solution comprising the therapeutic agent.
K10. The analyte sensor of K9, wherein at least one of the two or more spots of a therapeutic agent-containing polymer composition is formed by dispensing two layers of the polymer solution and a second spot of the two or more spots of a therapeutic agent-containing polymer composition is formed by dispensing three layers of the polymer solution.
K11. The analyte sensor of any one of K-K10, wherein the therapeutic agent is selected from group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.
K12. The analyte sensor of K11, wherein the therapeutic agent is an anti-inflammatory agent.
K13. The analyte sensor of K12, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
K14. The analyte sensor of any one of K1-K13, wherein the anti-inflammatory agent is dexamethasone, a derivative thereof, or a salt form thereof.
K14.1. The analyte sensor of any one of K-K14, wherein the therapeutic agent is in a range of 0.01 wt %-50 wt % based on the total weight of the polymer within the two or more spots of the therapeutic agent-containing polymer composition.
K15. The analyte sensor of any one of K-K14.1, wherein the therapeutic agent is in a range of 0.01 wt %-40 wt % based on the total weight of the polymer within the two or more spots of the therapeutic agent-containing polymer composition.
K16. The analyte sensor of any one of K-K15, wherein the two or more spots of the therapeutic agent-containing polymer composition comprises from about 0.01 μg to about 200 μg of the therapeutic agent.
K17. The analyte sensor of any one of K-K16, wherein the two or more spots of the therapeutic agent-containing polymer composition comprises from about 0.01 μg to about 20 μg of the therapeutic agent.
K18. The analyte sensor of any one of K-K17, wherein the two or more spots of the therapeutic agent-containing polymer composition comprises from about 0.01 μg to about 10 μg of the therapeutic agent.
K19. The analyte sensor of any one of K-K18, wherein the two or more spots of the therapeutic agent-containing polymer composition comprises from about 0.01 μg to about 5 μg of the therapeutic agent.
K20. The analyte sensor of any one of K-K19, wherein the analyte sensor is a subcutaneous sensor.
K21. The analyte sensor of any one of K-K20, wherein the analyte sensor is configured to detect glucose.
L. The present disclosure provides an analyte sensor comprising an in vivo portion configured to reside below a skin surface of a subject and in contact with interstitial fluid of the subject, wherein the in vivo portion comprises:
(i) a substrate comprising (a) a second region comprising an active area and (b) a first region distal to the second region;
(ii) a working electrode on the substrate comprising the active area;
(iii) a counter/reference electrode on the substrate;
(vi) a first polymer membrane coated on the first region and the second region including active area; and
(viii) a second polymer membrane coated on the first polymer membrane in the first region and not in the second region, wherein the second polymer membrane includes a therapeutic agent.
L1. The present disclosure provides an analyte sensor comprising an in vivo portion configured to reside below a skin surface of a subject and in contact with interstitial fluid of the subject, wherein the in vivo portion comprises:
(i) a substrate comprising (a) a second region comprising an active area and (b) a first region distal to the second region;
(ii) a working electrode on the substrate comprising the active area;
(iii) a counter/reference electrode on the substrate;
(iv) a second polymer membrane coated on the first region and not the second region, wherein the second polymer membrane includes a therapeutic agent; and
(v) a first polymer membrane coated on the first region and the second region including active area and on top of the second polymer membrane.
L2. The analyte sensor of L or L1, wherein the first polymer membrane is composed of a first polymer comprising a first copolymer selected from the group consisting of a polyvinylpyridine-based polymer, a polyvinylimidazole-based polymer, and a combination thereof.
L3. The analyte sensor of L or L1, wherein the first polymer membrane is composed of a first polymer comprising a first copolymer selected from the group consisting of a polyvinylpyridine-based polymer, a polyvinylimidazole-based polymer, and a combination thereof.
L4. The analyte sensor of L3, wherein the polyvinylpyridine-based copolymer is a polyvinylpyridine-co-polystyrene polymer.
L5. The analyte sensor of any one of L-L4, wherein the second polymer membrane is composed of a polymer selected from a polyvinylpyridine-based polymer, a polyvinylimidazole-based polymer, a polyacrylate-based polymer, a polyurethane-based polymer, a polyether urethane-based polymer, a silicone-based polymer, a derivative thereof, copolymers thereof and combinations thereof.
L6. The analyte sensor of L5, wherein the polyvinylpyridine-based polymer is is a polyvinylpyridine-co-polystyrene polymer.
L7. The analyte sensor of any one of L-L6, wherein the first polymer membrane is a mass-limiting membrane.
L7.1. The analyte sensor of any one of L-L7, wherein the second polymer membrane is a drug-eluting membrane.
L7.2. The analyte sensor of any one of L-L7.1, the therapeutic agent is an anti-inflammatory agent.
L8. The analyte sensor of L7.2, wherein the anti-inflammatory agent is selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.
L9. The analyte sensor of L7.2 or L8, wherein the anti-inflammatory agent is dexamethasone, a derivative thereof, or a salt form thereof.
L10. The analyte sensor of any one of L-L9, wherein the therapeutic agent is in a range of 0.01 wt %-50 wt % based on a total weight of the second polymer in the second polymer membrane.
L11. The analyte sensor of any one of L-L10, wherein the therapeutic agent is in a range of 0.01 wt %-40 wt % based on a total weight of the second polymer in the second polymer membrane.
L12. The analyte sensor of any one of L-L11, wherein second polymer membrane comprises from about 0.01 μg to about 200 μg of the therapeutic agent.
L13. The analyte sensor of any one of L-L12, wherein second polymer membrane comprises from about 0.01 μg to about 20 μg of the therapeutic agent.
L14. The analyte sensor of any one of L-L13, wherein second polymer membrane comprises from about 0.01 μg to about 10 μg of the therapeutic agent.
L15. The analyte sensor of any one of L-L14, wherein second polymer membrane comprises from about 0.01 μg to about 5 μg of the therapeutic agent.
L16. The analyte sensor of any one of L-L15, wherein the second region has a length of about 5 μm to about 50 μm.
L17. The analyte sensor of any one of L-L16, wherein the first region has a length of about 5 μm to about 50 μm.
L18. The analyte sensor of any one of L-L17, wherein the first region has a length of about 5 μm to about 20 μm.

### EXAMPLES

The presently disclosed subject matter will be better understood by reference to the following examples, which are provided as exemplary of the presently disclosed subject matter, and not by way of limitation.

### Example 1: Deposition of a Therapeutic Agent-Containing Polymer Composition onto a Counter Electrode Analyte Sensor

This example provides a method for depositing a therapeutic agent-containing polymer composition onto a counter electrode of an analyte sensor.

FIG. 12A provides images of exemplary analyte sensors that include a therapeutic agent-containing polymer composition and shows an exemplary method for applying a therapeutic agent-containing polymer composition onto the counter electrode of an analyte sensor. As shown in FIG. 12A, a therapeutic agent-containing polymer composition that includes a therapeutic agent such as dexamethasone was applied to the fabricated sensor tails prior to the sensor tails being laser cut from the larger material. The therapeutic agent-containing polymer composition was applied to the sensor tails as two spots, where one spot was applied two times and the second spot was applied three times. When the sensor tails were singulated (e.g., laser cut) from the surrounding material, the two spots on the isolated sensor tails resembled rectangular structures with rounded ends on the shorter side of the rectangle, as shown in FIG. 12A. An exemplary illustration of an analyte sensor with two spots on the counter electrode is shown in FIG. 12B. As shown in FIG. 12B, the two spots of a therapeutic agent-containing polymer composition are located on the counter electrode and can have differing thicknesses. The spots can then be covered by the mass transport limiting membrane (e.g., the 10Q5-01 membrane). For example, the spot located at the more distal region of the sensor tail had a thickness of 22.6 μm and was approximately two-times thicker than the spot located more proximally from the tip of the sensor tail that had a thickness of 40.1 μm. As shown in FIG. 12B, the therapeutic agent-containing polymer composition was not located on the same plane as the sensing layer located on the working electrode, e.g., the therapeutic agent-containing polymer composition was applied to the counter electrode that was located on the opposing side of the sensor tail that has the sensing layer.

### Example 2: Deposition of a Therapeutic Agent-Containing Polymer Composition onto an Analyte Sensor Using Drug-Loading Structure

This example provides a method for depositing a therapeutic agent-containing polymer composition onto an analyte sensor using drug-loading structures.

FIG. 5 illustrates images of an exemplary analyte sensor according to one or more embodiments of the present disclosure. FIG. 5A illustrates that an analyte sensor can include a sensor tail including an active sensing region 502 and a non-sensing region 504 including a drug-loading structure 508 filled with a therapeutic-agent-containing polymer composition (e.g., a therapeutic agent-containing polymer composition as described herein). FIG. 5B is a magnified view of the distal region of the sensor tail of FIG. 5A and shows the detailed structures of the sensor tail. FIG. 5C illustrates that an analyte sensor can include a sensor tail including an active sensing region 502 including a plurality of sensing spots 506 and a non-sensing region 504 including a drug-loading structure 508 filled with a therapeutic-agent-containing polymer composition e.g., a therapeutic agent-containing polymer composition as described herein). The detailed description of the drug-loading structure 508 filled with the therapeutic agent-containing polymer composition can refer to the drug-loading structure and the therapeutic agent-containing polymer composition (or therapeutic agent-containing polymer composition) disclosed elsewhere in the present disclosure, which will not be repeated herein for conciseness.

### Example 3: Deposition of a Therapeutic Agent-Containing Polymer Composition onto the Distal Tip of the In Vivo Portion of an Analyte Sensor

This example provides a method for depositing a therapeutic agent-containing polymer composition onto the distal end of an in vivo portion of an analyte sensor.

FIGS. 9, 10 and 11 show exemplary analyte sensors that include a therapeutic agent-containing polymer composition (512 in FIGS. 9 and 11 and 412 in FIG. 10) disposed upon the distal end of the sensor tail.

As shown in FIGS. 9 and 10, the therapeutic agent-containing polymer composition (512 in FIGS. 9 and 412 in FIG. 10) is disposed upon the mass transport limiting membrane (510 in FIGS. 9 and 410 in FIG. 10) but does not overlap with the sensing layer, which is shown as multiple sensing spots (408a-408f in FIG. 10). FIG. 13A shows a sensor tail of an exemplary analyte sensor of the present disclosure covered by a mass transporting limiting membrane that was dipped in a therapeutic agent-containing polymer composition that includes dexamethasone three times (“3 dips”). FIG. 13B shows a sensor tail of an exemplary analyte sensor of the present disclosure covered by a mass transporting limiting membrane that was dipped in a therapeutic agent-containing polymer composition that includes dexamethasone four times (“4 dips”). The sensor tails shown in FIGS. 13A-13B were generated by dipping a sensor tail in a mass transporting limiting membrane solution to coat the sensor tail followed by drying the solution to form the mass transporting limiting membrane. The most distal region of the sensor tail (that does not include the sensing layer) was then dipped in a solution of a therapeutic agent-containing polymer composition followed by drying the solution to form the therapeutic agent-containing polymer composition.

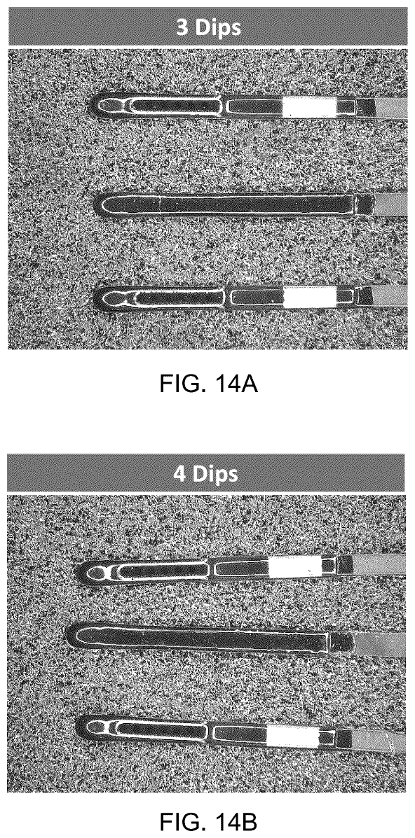

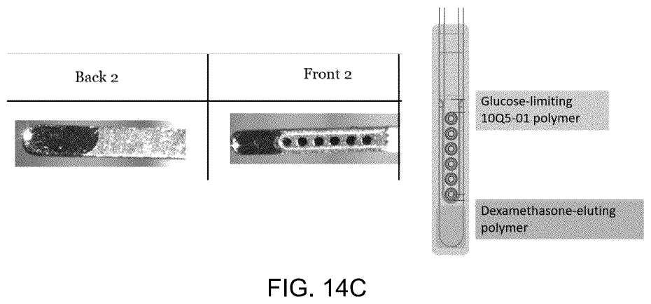

FIG. 11 shows an exemplary analyte sensor that includes a therapeutic agent-containing polymer composition (512) disposed upon the distal end of the sensor tail (e.g., on the substrate) and under the mass transporting limiting membrane 510. As shown in FIG. 11, the therapeutic agent-containing polymer composition (512) is located underneath the mass transport limiting membrane (510) but does not overlap with the sensing layer, which is shown as multiple sensing spots (508a-508f). FIG. 14A shows a sensor tail of an exemplary analyte sensor of the present disclosure that was dipped in a therapeutic agent-containing polymer composition that includes dexamethasone three times (“3 dips”) covered by a mass transporting limiting membrane (e.g., the 10Q5-01 mass transporting limiting membrane). FIG. 14B shows a sensor tail of an exemplary analyte sensor of the present disclosure that was dipped in a therapeutic agent-containing polymer composition that includes dexamethasone four times covered by a mass transporting limiting membrane (e.g., the 10Q5-01 mass transporting limiting membrane). FIG. 14C shows the front with the sensing layer and the back (e.g., counter electrode) of the sensor tail that includes a therapeutic agent-containing polymer composition located on the distal end of the sensor tail. The sensor tails shown in FIGS. 14A-14C were generated by dipping the most distal region of the sensor tail (that does not include the sensing layer) into a solution of a therapeutic agent-containing polymer composition followed by drying the solution to form the therapeutic agent-containing polymer composition.

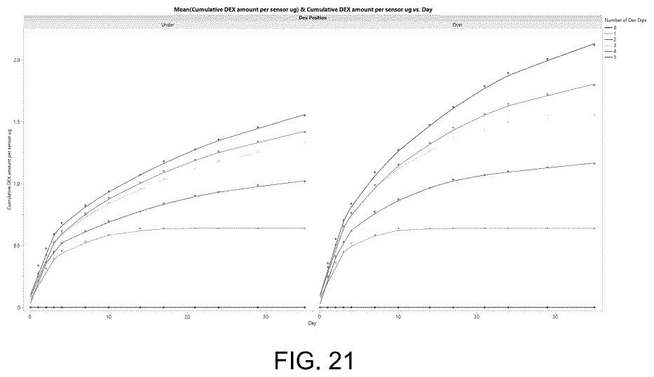

FIG. 21 shows the difference is elution rates of dexamethasone from analyte sensors that include the dexamethasone formulation above or underneath the mass transport limiting membrane and based on the number of dips. In certain embodiments, the elution rates and delivery rates of the dexamethasone can be determined by performing HPLC. The dexamethasone formulation applied to the distal tip of the sensor included about 40 wt % of dexamethasone, a polyvinylpyridine-co-styrene copolymer and Gly3. FIG. 22 shows the thicknesses of the Dex formulations when placed above or underneath the mass transport limiting membrane. As shown in FIG. 22, the dexamethasone formulation was thicker closer to the distal tip compared to the more proximal region of the Dex formulation.

Although the embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed and equivalents thereof. All patents, patent applications and articles disclosed herein are incorporated by reference in their entireties.

1-197. (canceled)

198. A method for manufacturing an analyte sensor comprising a therapeutic agent, the method comprising:
a) providing an analyte sensor comprising an in vivo portion configured to reside below a skin surface of a subject and in contact with interstitial fluid of the subject, wherein the in vivo portion comprises a second region comprising an active area and a first region distal to the second region;
b) preparing a first polymer solution comprising a first polymer or providing a first polymer solution comprising a first polymer;
c) preparing a second polymer solution comprising a second polymer and a therapeutic agent or providing a second polymer solution comprising a second polymer and a therapeutic agent;
d) contacting the in vivo portion with the first polymer solution to coat the first region and the second region to generate a first polymer membrane; and
e) contacting the first region with the second polymer solution to coat the first region to generate a second polymer membrane comprising the therapeutic agent, wherein the second region is free of the second polymer membrane.

199. The method of claim 198, wherein:
(i) contacting the in vivo portion with the first polymer solution comprises a screen printing, a rotary printing, a jetting, an aerosol deposition, a spray coating, a dip coating, a drop-casting, a spin coating or a brush coating process; and/or
(ii) contacting first region with the second polymer solution comprises a screen printing, a rotary printing, a jetting, an aerosol deposition, a spray coating, a dip coating, a drop-casting, a spin coating or a brush coating process.

200. The method of claim 198, wherein the therapeutic agent is selected from the group consisting of an antibiotic agent, an antiviral agent, an anti-inflammatory agent, an anti-cancer agent, an antiplatelet agent, an anticoagulant agent, a coagulant agent, an antiglycolytic agent and a combination thereof.

201. The method of claim 200, wherein the anti-inflammatory agent is dexamethasone, a derivative thereof, or a salt form thereof.

202. The method of claim 198, wherein the second polymer solution (i) further comprises a crosslinker, (ii) further comprises a solvent and/or (iii) has a viscosity of about 20 cP to about 250 cP.

203. The method of claim 202, wherein:
(i) the therapeutic agent is present in the second polymer solution in a range from about 0.01 wt % to about 50 wt % based on the total weight of the second polymer solution;
(ii) the second polymer is present in the second polymer solution in a range from about 0.01 wt % to about 50 wt % based on the total weight of the second polymer solution; and/or
(iii) the crosslinker is present in the second polymer solution in a range from about 0.01 wt % to about 50 wt % based on the total weight of the second polymer solution.

204. The method of claim 198, wherein:
(i) the therapeutic agent-containing polymer composition can continuously release the therapeutic agent at a drug delivery rate of about 0.01 μg/day to about 1 mg/day of the therapeutic agent;
(ii) the therapeutical agent is in a range of 0.01 wt %-50 wt % based on a total weight of the therapeutic agent-containing polymer composition; and/or
(iii) the therapeutic agent-containing polymer composition about 0.01 μg to about 100 μg of the therapeutic agent.

205. The method of claim 198, wherein the second polymer of the second polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.

206. The method of claim 205, wherein the polyvinylpyridine-based copolymer is a polyvinylpyridine-co-polystyrene polymer.

207. The method of claim 198, wherein:
(i) the first region of the analyte sensor is dipped into the second polymer solution one or more times to generate a second polymer membrane having a thickness between about 5 μm to about 40 μm; and/or
(ii) the in vivo portion of the analyte sensor is dipped into the first polymer solution one or more times to generate a first polymer membrane having a thickness between about 5 μm to about 40 μm.

208. The method of claim 198, wherein the first polymer of the first polymer solution is selected from the group consisting of a polyvinylpyridine-based polymer, a polyvinylimidazole-based polymer, a polyacrylate-based polymer, a polyurethane-based polymer, a polyether urethane-based polymer, a silicone-based polymer, a copolymer thereof, a derivative thereof, and a combination thereof.

209. The method of claim 208, wherein the polyvinylpyridine-based copolymer is a polyvinylpyridine-co-polystyrene polymer.

210. The method of claim 198, wherein the ratio of the thinnest point to the thickest point of the second polymer membrane is less than about 0.9.

211. An analyte sensor comprising an in vivo portion configured to reside below a skin surface of a subject and in contact with interstitial fluid of the subject, wherein the in vivo portion comprises:
(i) a substrate comprising (a) a second region comprising an active area and (b) a first region distal to the second region;
(ii) a working electrode on the substrate comprising the active area;
(iii) a counter/reference electrode on the substrate;
(iv) a second polymer membrane coated on the first region, wherein the second polymer membrane comprises a second polymer and a therapeutic agent; and
(v) a first polymer membrane comprising a first polymer coated on the first region and the second region,
wherein the second region is free of the second polymer membrane.

212. The analyte sensor of claim 211, wherein the first polymer membrane is coated on the second polymer membrane and/or wherein the second polymer membrane coated on the first polymer membrane.

213. The analyte sensor of claim 211, wherein the first polymer membrane is composed of a first polymer comprising a first copolymer selected from the group consisting of a polyvinylpyridine-based polymer, a polyvinylimidazole-based polymer, and a combination thereof; and/or wherein the second polymer membrane is composed of a polymer selected from a polyvinylpyridine-based polymer, a polyvinylimidazole-based polymer, a polyacrylate-based polymer, a polyurethane-based polymer, a polyether urethane-based polymer, a silicone-based polymer, a derivative thereof, copolymers thereof and combinations thereof.

214. The analyte sensor of claim 211, wherein the therapeutic agent is an anti-inflammatory agent selected from the group consisting of triamcinolone, betamethasone, dexamethasone, dexamethasone acetate, dexamethasone sodium phosphate, hydrocortisone, prednisone, methylprednisolone, fludrocortisone, acetylsalicylic acid, isobutylphenylpropanoic acid, a derivative thereof, a salt form thereof, and combinations thereof.

215. A method for fabricating an analyte sensor comprising an in vivo portion configured to reside below a skin surface of a subject and in contact with interstitial fluid of the subject, the method comprising:
a) patterning a plurality of first conductive layers on a substrate of the in vivo portion of the analyte sensor to generate a plurality of working electrodes;
b) patterning a plurality of second conductive layers on the substrate to generate a plurality of counter and/or reference electrodes;
c) forming one or more spots of a therapeutic agent-containing polymer composition on each counter and/or reference electrode of the plurality of counter and/or reference electrodes, wherein the therapeutic agent-containing polymer composition comprises a polymer and a therapeutic agent; and
d) singulating individual analyte sensors from the substrate, wherein each individual analyte sensor comprises at least one working electrode, at least one counter and/or reference electrode and the one or more spots of the therapeutic agent-containing polymer composition.

216. The method of claim 215, wherein two or more spots are formed on the counter and/or reference electrode, wherein at least two of the two spots comprise different amounts of the therapeutic agent.

217. The method of claim 215, wherein the difference in the amount of therapeutic agent between the two or more spots is about 10% or more and/or wherein at least two of the two spots comprise different thicknesses.

218. An analyte sensor prepared by the method of claim 215.

219. An analyte sensor comprising an in vivo portion configured to reside below a skin surface of a subject and in contact with interstitial fluid of the subject, wherein the in vivo portion comprises a non-sensing region and an active sensing region, wherein the non-sensing region surrounds the active sensing region and comprises a drug-loading structure filled with a therapeutical agent-containing polymer composition.

220. The analyte sensor of claim 219, wherein the drug-loading structure comprises at least one selected from a slot, a hole, a pore, a groove, and a depression.

221. The analyte sensor of claim 219, wherein the therapeutic agent-containing polymer composition comprises a polymer and a therapeutic agent.

222. A method of preparing the analyte sensor of claim 219, the method comprising:
a) removing at least a portion of the non-sensing region of the in vivo portion to form the drug-loading structure; and
b) filling the drug-loading structure with the therapeutic agent-containing polymer composition.

223. The method of claim 222, wherein the drug-loading structure comprises at least one selected from a slot, a hole, a pore, a groove, and a depression.

224. The method of claim 222, wherein the therapeutic agent-containing polymer composition comprises a polymer and a therapeutic agent.

225. An insertion device comprising:
a) an insertion tip configured to penetrate skin and coated with a therapeutic agent-containing polymer composition comprising a therapeutic agent and a polymer; and
b) an analyte sensor comprising:
i. a working electrode;
ii. an active area disposed upon the working electrode; and
iii. a membrane overcoating at least the active area.

226. The insertion device of claim 225, wherein the insertion tip is retractable.

227. The insertion device of claim 225, wherein the therapeutic agent-containing polymer composition overcoating the insertion tip is detachably attached to the insertion tip.

228. The insertion device of claim 225, wherein:
(i) the therapeutic agent-containing polymer composition continuously releases the therapeutic agent at a drug delivery rate of about 0.01 μg/day to about 1 mg/day of the therapeutic agent;
(ii) the therapeutical agent is in a range of 0.01 wt %-50 wt % based on a total weight of the therapeutic agent-containing polymer composition; and/or
(iii) wherein the therapeutic agent-containing polymer composition about 0.01 μg to about 100 μg of the therapeutic agent.

229. A method of inhibiting an immune response at an analyte sensor insertion, the method comprising:
a) introducing an analyte sensor into a tissue at the analyte sensor insertion site by use of the insertion device of claim 225; and
b) maintaining the therapeutic agent-containing polymer composition in the tissue following retraction of the sharp.

230. The method of claim 229, wherein the analyte sensor comprises a second therapeutic agent-containing polymer comprising a polymer and a therapeutic agent.
